(19) United States
(12) Patent Application Publication
(10) Pub. No.: US 2020/0102375 A1
(43) Pub. Date: Apr. 2, 2020

(54) Methods of Treating a Tauopathy

(71) Applicant: iPierian, Inc., South San Francisco, CA (US)

(72) Inventors: Irene Griswold-Prenner, South San Francisco, CA (US); Graham Parry, San Mateo, CA (US)

(21) Appl. No.: 16/398,097

(22) Filed: Apr. 29, 2019

Related U.S. Application Data
(63) Continuation of application No. 15/038,711, filed on May 23, 2016, now abandoned.
(60) Provisional application No. 61/909,965, filed on Nov. 27, 2013.

(51) Int. Cl. C07K 16/18

(52) U.S. Cl. CPC C07K 16/18; A61K 2039/505

(57) ABSTRACT

The present disclosure provides methods for treating a tauopathy (e.g., an acute tauopathy) in an individual by administering an anti-Tau antibody to the individual. Also provided are methods of treating traumatic brain injury and methods of treating stroke in an individual by administering an anti-Tau antibody to the individual.

### CROSS-REFERENCE TO RELATED APPLICATIONS

This application claims the benefit of priority of U.S. Provisional Application No. 61/909,965 (filed Nov. 27, 2013), which is incorporated herein by reference.

### BACKGROUND

The microtubule associated protein Tau is abundant in the central nervous system and is produced primarily by neurons. The primary function of Tau is to stabilize microtubules. Six Tau isoforms exist in the adult human brain; Tau isoforms are the products of alternative splicing of a single gene.

Tauopathies are a class of neurodegenerative diseases resulting from the pathological aggregation of Tau protein in so-called neurofibrillary tangles (NFT) in the brain. Some examples of tauopathies include frontotemporal dementia (FTD), Alzheimer's disease, progressive supranuclear palsy, corticobasal degeneration, and frontotemporal lobar degeneration.

There is a need in the art for methods of treating tauopathies.

### SUMMARY

The present disclosure provides methods for treating a tauopathy (e.g., an acute tauopathy) in an individual.

Accordingly, in one aspect, the methods of treating an acute tauopathy in an individual, are provided, the method comprising administering to the individual an anti-Tau antibody in an amount effective to reduce significantly the level of free Tau in an extracellular fluid of the individual.

In one embodiment, the anti-Tau antibody is effective to reduce significantly the level of free Tau in an extracellular fluid within 72 hours of administration of the anti-Tau antibody. In another embodiment, the anti-Tau antibody is effective to reduce significantly the level of free Tau in an extracellular fluid within 48 hours of administration of the anti-Tau antibody. In another embodiment, the anti-Tau antibody is effective to reduce significantly the level of free Tau in an extracellular fluid within 36 hours of administration of the anti-Tau antibody. In another embodiment, the anti-Tau antibody is effective to reduce significantly the level of free Tau in an extracellular fluid within 24 hours of administration of the anti-Tau antibody. In another embodiment, the anti-Tau antibody is effective to reduce significantly the level of free Tau in an extracellular fluid within 12 hours of administration of the anti-Tau antibody. In another embodiment, the anti-Tau antibody is effective to reduce significantly the level of free Tau in an extracellular fluid within 8 hours of administration of the anti-Tau antibody. In another embodiment, the anti-Tau antibody is effective to reduce significantly the level of free Tau in an extracellular fluid within 7 hours of administration of the anti-Tau antibody. In another embodiment, the anti-Tau antibody is effective to reduce significantly the level of free Tau in an extracellular fluid within 4 hours of administration of the anti-Tau antibody. In another embodiment, the anti-Tau antibody is effective to reduce significantly the level of free Tau in an extracellular fluid within 2 hours of administration of the anti-Tau antibody. In another embodiment, the anti-Tau antibody is effective to reduce significantly the level of free Tau in an extracellular fluid within 1 hour of administration of the anti-Tau antibody. In another embodiment, the anti-Tau antibody is effective to reduce significantly the level of free Tau in an extracellular fluid within 30 minutes of administration of the anti-Tau antibody.

In another embodiment, the anti-Tau antibody is effective to reduce the level of free Tau in an extracellular fluid by at least about 10%, In another embodiment, the anti-Tau antibody is effective to reduce the level of free Tau in an extracellular fluid by at least about 15%. In another embodiment, the anti-Tau antibody is effective to reduce the level of free Tau in an extracellular fluid by at least about 20%. In another embodiment, the anti-Tau antibody is effective to reduce the level of free Tau in an extracellular fluid by at least about 25%. In another embodiment, the anti-Tau antibody is effective to reduce the level of free Tau in an extracellular fluid by at least about 30%. In another embodiment, the anti-Tau antibody is effective to reduce the level of free Tau in an extracellular fluid by at least about 35%. In another embodiment, the anti-Tau antibody is effective to reduce the level of free Tau in an extracellular fluid by at least about 40%, In another embodiment, the anti-Tau antibody is effective to reduce the level of free Tau in an extracellular fluid by at least about 45%. In another embodiment, the anti-Tau antibody is effective to reduce the level of free Tau in an extracellular fluid by at least about 50%. In another embodiment, the anti-Tau antibody is effective to reduce the level of free Tau in an extracellular fluid by at least about 55%. In another embodiment, the anti-Tau antibody is effective to reduce the level of free Tau in an extracellular fluid by at least about 60%. In another embodiment, the anti-Tau antibody is effective to reduce the level of free Tau in an extracellular fluid by at least about 65%. In another embodiment, the anti-Tau antibody is effective to reduce the level of free Tau in an extracellular fluid by at least about 70%. In another embodiment, the anti-Tau antibody is effective to reduce the level of free Tau in an extracellular fluid by at least about 75%. In another embodiment, the anti-Tau antibody is effective to reduce the level of free Tau in an extracellular fluid by at least about 80%. In another embodiment, the anti-Tau antibody is effective to reduce the level of free Tau in an extracellular fluid by at least about 85%. in another embodiment, the anti-Tau antibody is effective to reduce the level of free Tau in an extracellular fluid by at least about 90%.

In another embodiment, the anti-Tau antibody is effective to reduce the level of free Tau in an extracellular fluid to an undetectable level. In another embodiment, the anti-Tau antibody is effective to reduce the level of free Tau in an extracellular fluid to a normal level. In another embodiment, the reduced level of free Tau is maintained for a period of time of at least 2 hours following administration of the anti-Tau antibody. In another embodiment, the reduced level of free Tau is maintained for a period of time of at least 5 hours following administration of the anti-Tau antibody. In another embodiment, the reduced level of free Tau is maintained for a period of time of at least 10 hours following administration of the anti-Tau antibody. In another embodiment, the reduced level of free Tau is maintained for a period of time of at least 24 hours following administration of the anti-Tau antibody. In another embodiment, the reduced level of free Tau is maintained for a period of time of at least 7 days following administration of the anti-Tau antibody. In some cases, the reduced level of free Tau is maintained for a period of time of at least 2 weeks following administration of the anti-Tau antibody.

In one embodiment, the extracellular fluid is plasma. In another embodiment, the extracellular fluid is cerebrospinal fluid. In another embodiment, the extracellular fluid is interstitial fluid. In another embodiment, the extracellular fluid is blood.

The anti-Tau antibody can be administered by any suitable means. For example, the anti-Tau antibody can be administered by subcutaneous administration, by intrathecal administration, or by intravenous administration.

In one embodiment, the anti-Tau antibody is administered in an amount of from about 0.1 mg/kg body weight to about 50 mg/kg body weight. In another embodiment, the anti-Tau antibody is administered in a single bolus injection.

In another embodiment, multiple doses of the anti-Tau antibody are administered (e.g., 2, 3, 4, 5, 6, 7, 8, or 9 doses). In one embodiment, where multiple doses of the anti-Tau antibody are administered, any two doses of the anti-Tau antibody are administered within 3 days or more of one another. In another embodiment, where multiple doses of the anti-Tau antibody are administered, any two doses of the anti-Tau antibody are administered within 5 days or more of one another. In another embodiment, where multiple doses of the anti-Tau antibody are administered, any two doses of the anti-Tau antibody are administered within 7 days or more of one another. In another embodiment, where multiple doses of the anti-Tau antibody are administered, any two doses of the anti-Tau antibody are administered within 2 weeks or more of one another. In another embodiment, where multiple doses of the anti-Tau antibody are administered, any two doses of the anti-Tau antibody are administered within 4 weeks or more of one another. In another embodiment, where multiple doses of the anti-Tau antibody are administered, any two doses of the anti-Tau antibody are administered within 2 months or more of one another.

The present disclosure also provides a method of treating an acute tauopathy in an individual, the method comprising administering to the individual an anti-Thu antibody in an amount effective to provide for a minimal concentration of the anti-Tau antibody in cerebrospinal fluid (CSF) of the individual. In one embodiment, the minimal concentration of anti-Tau antibody in the CSF is achieved within 1 hour of administration of the anti-Tau antibody. In another embodiment, the minimal concentration of anti-Tau antibody in the CSF at least 20 ng/ml. In another embodiment, the minimal concentration of anti-Tau antibody in the CSF at least 30 ng/ml. In another embodiment, the minimal concentration of anti-Tau antibody in the CSF provides for a molar ratio of the anti-Tau antibody to Tau in the CSF of at least 2:1. In another embodiment, the minimal concentration of anti-Tau antibody in the CSF provides for a molar ratio of the anti-Tau antibody to Tau in the CSF of at least 2.5:1.

In any of the embodiments described above or herein, the acute tauopathy can be traumatic brain injury (e.g., diffuse axonal injury, concussion, contusion, Coup-Contrecoup injury, Second Impact Syndrome, penetrating injury, Shaken Baby Syndrome, and Locked In Syndrome. In any of the embodiments described above or herein, the acute tauopathy can be stroke. In any of the embodiments described above or herein, the acute tauopathy can be chronic traumatic encephalopathy.

The present disclosure further provides a method of treating traumatic brain injury in an individual, the method comprising administering to the individual an anti-Tau antibody in an amount effective to reduce significantly the level of free Tau in an extracellular fluid of the individual. In some cases, the antibody is administered within 48 hours of the traumatic brain injury. In some cases, the antibody is administered in a single dose. In some cases, the antibody is administered in multiple doses. In some cases, the antibody is administered every week, every 2 weeks, every 4 weeks, every 6 weeks, every 8 weeks, every 3 months, or every 6 months.

The present disclosure also provides a method of treating stroke in an individual, the method comprising administering to the individual an anti-Tau antibody in an amount effective to reduce significantly the level of free Tau in an extracellular fluid of the individual. In some cases, the antibody is administered within 48 hours of the stroke. In some cases, the antibody is administered in a single dose. In some cases, the antibody is administered in multiple doses. In some cases, the antibody is administered every week, every 2 weeks, every 4 weeks, every 6 weeks, every 8 weeks, every 3 months, or every 6 months.

The present disclosure further provides a method of treating an acute tauopathy in an individual, the method comprising administering to the individual an anti-Tau antibody in an amount effective to reduce significantly the level of free Tau in an extracellular fluid of the individual for a period of time sufficient to reduce AP levels in the extracellular fluid. In one embodiment, the antibody is administered in a single dose. In another embodiment, the antibody is administered in multiple doses. In another embodiment, the antibody is administered every week, every 2 weeks, every 4 weeks, every 6 weeks, every 8 weeks, every 3 months, or every 6 months.

In any one of the embodiments described above or herein, the level of Aja is reduced significantly within a period of time of from about 5 days to about 15 days after administration of the anti-Tau Ab.

Any suitable anti-Tau antibody can be used in the methods described herein. An exemplary anti-Tau antibody is hu-IPN002 (also known as IPN007 and IPN002 Variant 2) comprising heavy and light chains having the sequences shown in SEQ ID NOs:37 and 41, respectively, or antigen binding fragments and variants thereof. hu-IPN002 is a humanized immunoglobulin (IgG4) monoclonal antibody that binds to extracellular Tau.

In one embodiment, the antibody comprises the heavy and light chain CDRs or variable regions of hu-IPN002. Accordingly, in one embodiment, the antibody comprises the CDR1, CDR2, and CDR3 domains of the VH region of hu-IPN002 having the sequence set forth in SEQ ID NO:37, and the CDR1, CDR2 and CDR3 domains of the VL region of hu-IPN002 having the sequence set forth in SEQ ID NO:41. In another embodiment, the antibody comprises heavy chain CDR1, CDR2 and CDR3 domains having the sequences set forth in SEQ NOs: 10, 11, and 12, respectively, and light chain CDR1, CDR2 and CDR3 domains having the sequences set forth in SEQ ID NOs:7, 8, and 9, respectively. In another embodiment, the antibody comprises VH and/or VL regions having the amino acid sequences set forth in SEQ ID NO:37 and/or SEQ ID NO: 41, respectively. In another embodiment, the antibody comprises the heavy chain variable (VH) and/or light chain variable (VL) regions encoded by the nucleic acid sequences set forth in SEQ ID NO:29 and/or SEQ ID NO:33, respectively. In another embodiment, the antibody competes for binding with, and/or binds to the same epitope on Tau as, the above-mentioned antibodies. In another embodiment, the antibody has at least about 90% variable region amino acid sequence identity with the above-mentioned antibodies (e.g., at least about 90%, 95% or 99% variable region identity with SEQ ID NO:37 or SEQ ID NO:41).

In another embodiment, the anti-Tau antibody that is administered can specifically bind an epitope within amino acids 1-158 of 2N4R Tau. In another embodiment, the anti-Tau antibody that is administered can specifically bind an epitope within amino acids 2-18 of Tau. In another embodiment, the anti-Tau antibody that is administered can specifically bind an epitope within amino acids 7-13 or within amino acids 25-30 of Tau. In another embodiment, the anti-Tau antibody that is administered can specifically bind an epitope within amino acids 15-24 of Tau. In another embodiment, the anti-Tau antibody that is administered can specifically bind an epitope within amino acids 28-126 of 2N4R. Tau. In another embodiment, the anti-Tau antibody that is administered can specifically bind an epitope within amino acids 150-158 of 2N4R Tau. In another embodiment, the anti-Tau antibody that is administered can bind a linear epitope. In another embodiment, the anti-Tau antibody that is administered can bind an epitope that is within a Tau polypeptide having at least 95% amino acid sequence identity to the eTau4 amino acid sequence depicted in FIG. 9.

In another embodiment, the anti-Tau antibody that is administered can be an anti-Tau antibody that competes for binding with an antibody that comprises: a) a light chain region comprising: i) a VL CDR1 comprising an amino acid sequence of SEQ ID NO:1 or SEQ ID NO:7; (ii) a VL CDR2 comprising an amino acid sequence of SEQ ID NO:2 or SEQ ID NO:8; and (iii) a VL CDR3 comprising an amino acid sequence of SEQ ID NO or SEQ ID NO:9; and b) a heavy chain region comprising: (i) a VH CDR1 comprising an amino acid sequence of SEQ ID NO:4 or SEQ II) NO:10; (ii) a VII CDR2 comprising an amino acid sequence of SEQ ID NO:5 or SEQ ID NO:11; and (iii) a VH CDR3 comprising an amino acid sequence of SEQ ID NO:6 or SEQ ID NO:12.

In another embodiment, the anti-Tau antibody is an anti-Tau antibody that comprises: a) a light chain region comprising: i) a VL CDR1 comprising an amino acid sequence of SEQ ID NO:1 or SEQ ID NO:7; (ii) a VL CDR2 comprising an amino acid sequence of SEQ ID NO:2 or SEQ ID NO:8; and (iii) a VL CDR3 comprising an amino acid sequence of SEQ ID NO:3 or SEQ ID NO:9; and b) a heavy chain region comprising: (i) a VH CDR1 comprising an amino acid sequence of SEQ ID NO:4 or SEQ ID NO:10; (ii) a VH CDR2 comprising an amino acid sequence of SEQ ID NO:5 or SEQ ID NO:11; and (iii) a VH CDR3 comprising an amino acid sequence of SEQ ID NO:6 or SEQ ID NO:12.

In another embodiment, the anti-Tau antibody is an anti-Tau antibody that binds specifically to the epitope independently of phosphorylation of amino acids within the epitope. In another embodiment, the anti-Tau antibody is a humanized anti-Tau antibody.

### BRIEF DESCRIPTION OF THE DRAWINGS

FIG. 1 depicts levels of IPN002 in plasma and in cerebrospinal fluid (CSF) following a single injection of IPN002 into cynomolgus monkeys.

FIG. 2 depicts the effect of IPN002 on levels of Tau in CSF of cynomolgus monkeys treated with IPN002.

FIG. 3 depicts the effect of IPN002 on levels of Aβ protein in CSF of cynomolgus monkeys treated with IPN002.

FIGS. 4A and 4B depict levels of hu-IPN002 in serum of non-human primates treated with 5 mg/kg (FIG. 4A) or 20 mg/kg (FIG. 4B) of the hu-IPN002.

FIGS. 5A and 5B depict levels of hu-IPN002 in CSF of non-human primates treated with 5 mg/kg (FIG. 5A) or 20 mg/kg (FIG. 5B) of the hu-IPN002.

FIG. 6 provides a summary of the pharmacokinetic data depicted in FIGS. 4A and 4B and FIGS. 5A and 5B.

FIG. 7 depicts the effect of administration of hu-IPN002 antibody on the level of free Tau in CSF of non-human primates treated with 5 mg/kg or 20 mg/kg of the hu-IPN002 antibody.

FIG. 8 depicts the effect of administration of hu-IPN002 antibody on the level of Aβ40 in CSF of non-human primates treated with 5 mg/kg or 20 mg/kg of the hu-IPN002 antibody.

FIG. 9 provides an amino acid sequence of 2N4R Tau (SEQ ID NO: 72) aligned with eTau4 (SEQ ID NO: 71).

FIG. 10 depicts the presence of Tau fragments in CSF from individuals with likely chronic traumatic encephalopathy.

FIGS. 11A and 11B provide amino acid sequences of IPN001 VH (FIG. 11A) and VL (FIG. 11B). Complementarity-determining regions (CDRs) are in bold text and underlined.

FIGS. 12A and 12B provide amino acid sequences of IPN002 VH (FIG. 12A) and VL (FIG. 12B), Complementarity-determining regions (CDRs) are in bold text and underlined.

FIG. 13 depicts an amino acid sequence of humanized IPN002 VH variant 1; and a nucleotide sequence encoding the amino acid sequence.

FIG. 14 depicts an amino acid sequence of humanized IPN002 VH variant 2; and a nucleotide sequence encoding the amino acid sequence.

FIG. 15 depicts an amino acid sequence of humanized IPN002 VII variant 3; and a nucleotide sequence encoding the amino acid sequence.

FIG. 16 depicts an amino acid sequence of humanized IPN002 VH variant 4; and a nucleotide sequence encoding the amino acid sequence.

FIG. 17 depicts an amino acid sequence of humanized IPN002 Vκ variant 1; and a nucleotide sequence encoding the amino acid sequence.

FIG. 18 depicts an amino acid sequence of humanized IPN002 Vκ variant 2; and a nucleotide sequence encoding the amino acid sequence.

FIG. 19 depicts an amino acid sequence of humanized IPN002 Vκ variant 3; and a nucleotide sequence the amino acid sequence.

FIG. 20 depicts an amino acid sequence of humanized IPN002 Vκ variant 4; and a nucleotide sequence encoding the amino acid sequence.

FIG. 21 provides amino acid sequences of various extracellular Tau polypeptides (SEQ ID NOS 73-78, respectively, in order of appearance)

FIG. 22 depicts levels of hu-IPN002 in serum of non-human primates treated with 5 mg/kg or 20 mg/kg of hu-IPN002 up to 56 days post-treatment.

FIG. 23 depicts levels of hu-IPN002 in CSF of non-human primates treated with 5 mg/kg or 20 mg/kg of hu-IPN002 up to 56 days post-treatment.

FIG. 24 depicts free Tau levels in CSF of non-human primates treated with 5 mg/kg or 20 mg/kg of hu-IPN002, up to 56 days post-treatment.

FIG. 25 depicts Aβ40 levels in CSF of non-human primates treated with 5 mg/kg or 20 mg/kg of hu-IPN002, up to 56 days post-treatment.

FIG. 26 depicts levels of hu-IPN002 in serum of non-human primates treated with 0.5 mg/kg, 2 mg/kg, 5 mg/kg, or 20 mg/kg of hu-IPN002, up to 57 days post-treatment.

FIG. 27 depicts levels of hu-IPN002 in CSF of non-human primates treated with 0.5 mg/kg, 2 mg/kg, 5 mg/kg, or 20 mg/kg of hu-IPN002, up to 57 days post-treatment.

FIG. 28 depicts free Tau levels in CSF in non-human primates treated with 0.5 mg/kg, 2 mg/kg, 5 mg/kg, or 20 mg/kg of hu-IPN002, up to 57 days post-treatment.

FIGS. 29A-29B compare free Tau CSF levels in non-human primates treated with 0.5 mg/kg, 2 mg/kg, 5 mg/kg, or 20 mg/kg of hu-IPN002, up to 57 days post-treatment.

FIGS. 30A-30B compare Aβ40 levels in CSF in non-human primates treated with 0.5 mg/kg, 2. mg/kg, 5 mg/kg, or 20 mg/kg of hu-IPN002, up to 57 days post-treatment, as assessed using an in-house assay.

FIG. 31A-31B compare Aβ40 levels in CSF in non-human primates treated with 0.5 mg/kg, 2 mg/kg, 5 mg/kg, or 20 mg/kg of hu-IPN002, up to 57 days post-treatment, as assessed using a Millipore assay.

FIG. 32 depicts day 1 levels of hu-IPN002 in serum of non-human primates treated with a multiple dose regimen of hu-IPN002.

FIG. 33 depicts day 29 levels of hu-IPN002 in serum of non-human primates treated with a multiple dose regimen of hu-IPN002.

FIG. 34 depicts day 57 levels of hu-IPN002 in serum of non-human primates treated with a multiple dose regimen of hu-IPN002.

FIG. 35 depicts day 1 levels of hu-IPN002 in CSF of non-human primates treated with a multiple dose regimen of hu-IPN002.

FIG. 36 depicts day 29 levels of hu-IPN002 in CSF of non-human primates treated with a multiple dose regimen of hu-IPN002.

FIG. 37 depicts day 57 levels of hu-IPN002 in CSF of non-human primates treated with a multiple dose regimen of hu-IPN002.

FIG. 38 depicts free Tau levels in CSF of non-human primates treated with a multiple dose regimen of hu-IPN002, up to day 112.

FIG. 39 depicts free Tau levels in CSF of non-human primates treated with a multiple dose regimen of 20 mg/kg of hu-IPN002 on days 1, 29, and 57 (Group 2) compared to the control (Group 1), up to day 169.

FIG. 40 depicts Aβ40 levels in CSF of non-human primates treated with a multiple dose regimen of hu-IPN002, up to day 112.

FIG. 41 depicts the simulated serum and CSF concentrations of free hu-IPN002 and free eTau in humans after a 10 mpk IV infusion.

FIG. 42 depicts the predicted human plasma concentration-time profile following a 700 mg Q4W (dashed line) and 700 mg loading dose+280 mg Q4W dosing regimen (dotted line).

FIG. 43 depicts the predicted human plasma eTau concentration-time profile following a 700 mg Q4W (dashed line) and 700 mg loading dose+280 mg Q4W (dotted line) dosing regimen.

### DEFINITIONS

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, bi-specific antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)2, and or other antibody fragments that retain specific binding to antigen, and monoclonal antibodies. An antibody may be monovalent or bivalent.

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a CDR derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)), regarding single chain antibodies.

For example, humanized immunoglobulins can be produced using synthetic and/or recombinant nucleic acids to prepare genes (e.g., cDNA) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. For example, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993)). “Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng 8(10): 1057-1062 (1995)); single-chain antibody molecules; and multi specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment which contains a complete antigen-recognition and -binding site, This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgG, and IgM, and several of these may be further divided into subclasses (isotypes), e.g., IgG-1, IgG2, IgG3, IgG4, IgA, and IgA2.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “affinity” refers to the equilibrium constant for the reversible binding of two agents (e.g., an antibody and an antigen) and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. A suitable anti-Tau antibody binds specifically to an epitope within a Tau polypeptide. Non-specific binding would refer to binding with an affinity of less than about 10-7 M, e.g., binding with an affinity of 10-6 M, 10-5 M, 10-4 M, etc.

As used herein, the term “CDR” or “complementarity determining region” is intended to mean the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by CDRs.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In some embodiments, the antibody will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated antibody includes the antibody in situ within recombinant cells since at least one component of the antibody's natural environment will not be present. In some instances, isolated antibody will be prepared by at least one purification step.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from Tau are tested for reactivity with the given anti-Tau antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

Other techniques include, for example, epitope mapping methods, such as, x-ray analyses of crystals of antigen:antibody complexes which provides atomic resolution of the epitope. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

The term “epitope mapping” refers to the process of identification of the molecular determinants for antibody-antigen recognition.

The term “binds to the same epitope” with reference to two or more antibodies means that the antibodies bind to the same, overlapping or encompassing continuous or discontinuous segments of amino acids. Those of skill in the art understand that the phrase “binds to the same epitope” does not necessarily mean that the antibodies bind to exactly the same amino acids. The precise amino acids to which the antibodies bind can differ. For example, a first antibody can bind to a segment of amino acids that is completely encompassed by the segment of amino acids bound by a second antibody. In another example, a first antibody binds one or more segments of amino acids that significantly overlap the one or more segments bound by the second antibody. For the purposes herein, such antibodies are considered to “bind to the same epitope.”

Accordingly, also, encompassed by the present invention are antibodies that bind to an epitope on Tau which comprises all or a portion of an epitope recognized by the particular antibodies described herein (e.g., the same or an overlapping region or a region between or spanning the region).

Also encompassed by the present invention are antibodies that compete for binding to Tau with the antibodies described herein. Antibodies that compete for binding can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common antigen, such as Tau. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin ETA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin ETA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more. The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates equines, bovines, ovines, porcines, caprines), etc.

A “therapeutically effective amount” or “efficacious amount” refers to the amount of an anti-Tau antibody that, when administered to a mammal or other subject for treating a disease, is sufficient to effect such treatment for the disease. The “therapeutically effective amount” will vary depending on the anti-Tau antibody, the disease and its severity and the age, weight, etc., of the subject to be treated.

As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., the anti-Tau antibody).

As used herein, a “body surface area (BSA)-based dose” refers to a dose (e.g., of the anti-Tau antibody) that is adjusted to the body-surface area (BSA) of the individual patient. A BSA-based dose may be provided as mg/kg body weight. Various calculations have been published to arrive at the BSA without direct measurement, the most widely used of which is the Du Bois formula (see Du Bois D, Du Bois E F (June 1916) Archives of Internal Medicine 17 (6): 863-71; and Verbraecken, J. et al. (April 2006). Metabolism—Clinical and Experimental 55 (4): 515-24). Other exemplary BSA formulas include the Mosteller formula (Mosteller R D. N Engl J Med., 1987; 317:1098), the Haycock formula (Haycock G B, et al., J Pediatr 1978, 93:62-66), the Gehan and George formula (Gehan E A, George S L, Cancer Chemother Rep 1970, 54:225-235), the Boyd formula (Current, J D (1998), The Internet Journal of Anesthesiology 2 (2); and Boyd, Edith (1935), University of Minnesota. The Institute of Child Welfare, Monograph Series, No. x. London: Oxford University Press), the Fujimoto formula (Fujimoto 5, et al., Nippon Eiseigaku Zasshi 1968; 5:443-50), the Takahira formula (Fujimoto S, et al., Nippon Eiseigaku Zasshi 1968; 5:443-50), and the Schlich formula (Schlich E, et al., Emährungs Umschau 2010; 57:178-183). A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, blood fractions such as plasma and serum, and the like.

The term “acute tauopathy,” as used herein, refers to a disease, disorder, or condition associated with sudden onset of abnormally elevated Tau (e.g., elevated compared to a normal, control level of Tau) in extracellular fluid (e.g., cerebrospinal fluid (CSF), interstitial fluid (ISF), blood, or a blood fraction (e.g., a blood fraction such as serum or plasma) of a subject, e.g., elevated Tau in extracellular fluid following an insult associated with physical disturbance to a subject's brain and/or associated tissues of the central nervous system. Such insult is generally followed by elevation of Tau in extracellular fluid (e.g., CSF, ISF, blood, and/or blood fractions (e.g., plasma)) within a relatively short period of time, e.g., within weeks or months (or a shorter time period). Examples of such insults include, but are not necessarily limited to, physical trauma (e.g., head injury) and stroke. Non-limiting examples of acute tauopathies are stroke, chronic traumatic encephalopathy, traumatic brain injury, concussion, seizures, epilepsy (e.g., Dravet Syndrome (also known as Severe Myoclonic Epilepsy of Infancy (SMEI)), and acute lead encephalopathy.

The phrase “traumatic brain injury” (also known as “TBI”) is a form of acquired brain injury, which occurs when a trauma causes damage to the brain (e.g., an injury to the brain caused by an external force). For example, TBI can result when the head suddenly and violently hits an object (e.g., during a fall, car accident, sporting event, or any number of different ways) or when an object pierces the skull and enters brain tissue. Both types of TBI can result in bruised brain tissue, bleeding inside the brain, large or small lacerations in the brain, and/or nerve damage due to shearing forces. The brain can also experience a number of secondary types of damage, such as swelling, fever, seizures, or an imbalance of neurological chemicals. Symptoms of TBI can be mild, moderate, or severe, depending on the extent of the damage to the brain. A person with a mild TBI may remain conscious or may experience a loss of consciousness for a few seconds or minutes. Other symptoms of mild TBI include headache, confusion, lightheadedness, dizziness, blurred vision or tired eyes, ringing in the ears, bad taste in the mouth, fatigue or lethargy, a change in sleep patterns, behavioral or mood changes, and trouble with memory, concentration, attention, or thinking. A person with a moderate or severe TBI may show these same symptoms, but may also have a headache that gets worse or does not go away, repeated vomiting or nausea, convulsions or seizures, an inability to awaken from sleep, dilation of one or both pupils of the eyes, slurred speech, weakness or numbness in the extremities, loss of coordination, and increased confusion, restlessness, or agitation. Examples of TBI include, but are not limited to, diffuse axonal injury, concussion, contusion, Coup-Contrecoup injury, Second Impact Syndrome, penetrating injury, Shaken Baby Syndrome, and Locked In Syndrome.

“Chronic tauopathy” is used herein to generally refer to a condition associated with a gradual onset of elevated. Tau in extracellular fluid of a subject, e.g., accumulation of Tau in extracellular fluid (e.g., CSF, ISF, blood, and/or blood fractions (e.g., plasma)) over a relatively longer period of time, e.g., multiple years, e.g., decades. Chronic tauopathies include, but are not necessarily limited to, Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tang only dementia, and multi-infarct dementia.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an anti-Tau antibody” includes a plurality of such anti-Tau antibodies and reference to “the tauopathy” includes reference to one or more tauopathies and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods for treating a tauopathy in an individual.

Methods of Treating a Tauopathy

The present disclosure provides methods of treating a tauopathy, e.g., an acute tauopathy. The methods generally involve administering to an individual in need thereof an effective amount of an anti-Tau antibody, or a pharmaceutical composition comprising an anti-Tau antibody. In some cases, the anti-Tau antibody specifically binds an epitope within an N-terminal region of Tau. In some cases, the anti-Tau antibody specifically binds an epitope within the N-terminal region of extracellular Tau (eTau). In some cases, the antibody is humanized. In some cases, the extracellular fluid is cerebrospinal fluid (CSF), interstitial fluid (ISF), blood, or a blood fraction (e.g., a blood fraction such as serum or plasma). In some cases, the tauopathy is an acute tauopathy such as stroke, chronic traumatic encephalopathy, traumatic brain injury, concussion, seizures, epilepsy (e.g., Dravet Syndrome (also known as Severe Myoclonic Epilepsy of Infancy (SMEI)), and acute lead encephalopathy, and acute lead encephalopathy. As described by Gheyara et al., Tau reduction may be of therapeutic benefit in Dravet syndrome and other intractable genetic epilepsies (Ann Neurol. 2014 September; 76(3):443-56). Accordingly, the methods described herein may be useful in treating any acute tauopathy, including, for example, epilepsy (e.g., Dravet Syndrome).

In some cases, the level of free Tau is reduced. “Free Tau” refers to a Tau polypeptide that is not bound to an anti-Tau antibody. In one embodiment, the free Tau is extracellular Tau (eTau). Total Tau includes free Tau and Tau that is bound to an anti-Tau antibody. In some cases, the level of total Tau is reduced. In some cases, the level of bound Tau (Tau bound to an anti-Tau antibody) in an extracellular fluid is increased.

The present disclosure provides methods of treating a tauopathy (e.g., an acute tauopathy) in an individual, the method comprising administering to the individual an anti-Tau antibody in an amount effective to reduce significantly the level of Tau (e.g., total Tau and/or free Tau) in an extracellular fluid (e.g., CSF, ISF, blood, or a blood fraction (e.g., serum or plasma)) in the individual. In some embodiments, the level of Tau (e.g., total Tau and/or free Tau) is significantly reduced within 36 hours of administration of the anti-Tau antibody. For example, in some cases, an effective amount of an anti-Tau antibody is an amount that is effective to reduce significantly the level of Tau (e.g., total Tau and/or free Tau) in an extracellular fluid within 48 hours, 36 hours, within 24 hours, within 12 hours, within 8 hours, within 4 hours, within 2 hours, within 1 hour, within 30 minutes, within 15 minutes, or within 5 minutes, of administration of the anti-Tau antibody. For example, in some cases, an effective amount of an anti-Tau antibody is an amount that is effective to reduce significantly the level of Tau (e.g., total Tau and/or free Tau) in an extracellular fluid within from 5 minutes to about 10 minutes, from about 10 minutes to about 15 minutes, from about 15 minutes to about 30 minutes, from about 30 minutes to about 1 hour, from about 1 hour to about 2 hours, from about 2 hours to about 4 hours, from about 4 hours to about 8 hours, from about 8 hours to about 12 hours, from about 12 hours to about 24 hours, from about 24 hours to about 36 hours, from about 24 to about 48 hours, or from about 36 hours to about 48 hours.

A significant reduction in the level of Tau (e.g., total Tau and/or free Tau) in an extracellular fluid (e.g., CSF, ISF, blood, or a blood fraction (e.g., serum or plasma)) of an individual is an at least 10% reduction, an at least 15% reduction, an at least 20% reduction, an at least 25% reduction, an at least 30% reduction, an at least 40% reduction, an at least 45% reduction, an at least 50% reduction, an at least 75% reduction, an at least 80% reduction, an at least 85% reduction, an at least 90% reduction, an at least 95% reduction, or a greater than 90% reduction. In some embodiments, the level of Tau (e.g., total Tau and/or free Tau) in an extracellular fluid is reduced to a normal, control level (e.g., about 100 pg/ml). In some embodiments, the level of Tau (e.g., total Tau and/or free Tau) in an extracellular fluid is reduced to an undetectable level. In some cases, the extracellular fluid is CSF. In other cases, the extracellular fluid is interstitial fluid (ISF). In other cases, the extracellular fluid is plasma. In other cases, the extracellular fluid is whole blood. In other cases, the extracellular fluid is serum.

The present disclosure provides a method of treating a tauopathy (e.g., an acute tauopathy) in an individual. The method generally involves administering to the individual an anti-Tau antibody in an amount effective to reduce significantly the level of Tau (e.g., total Tau and/or free Tau) in an extracellular fluid (e.g., CSF, ISF, blood, or a blood fraction (e.g., serum or plasma)) of the individual.

A significant reduction in the level of Tau (e.g., total Tau and/or free Tau) in an extracellular fluid of an individual is an at least 10% reduction, an at least 15% reduction, an at least 20% reduction, an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction, an at least 80% reduction, an at least 85% reduction, an at least 90% reduction, an at least 95% reduction, or a greater than 90% reduction. In some embodiments, the level of Tau (e.g., total Tau and/or free Tau) in an extracellular fluid is reduced to a normal, control level (e.g., about 100 pg/ml). In some embodiments, the level of Tau (e.g., total Tau and/or free Tau) in an extracellular fluid is reduced to an undetectable level. In some cases, the extracellular fluid is CSF. In other cases, the extracellular fluid is interstitial fluid (ISF). In other cases, the extracellular fluid is plasma. In other cases, the extracellular fluid is serum. In other cases, the extracellular fluid is whole blood.

In some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid (e.g., CSF, ISF, blood, or a blood fraction (e.g., serum or plasma)) of the individual, where the anti-Tau antibody is effective to reduce significantly the level of Tau (e.g., total Tau and/or free Tau) in an extracellular fluid within 48 hours of administration of the anti-Tau antibody. For example, in some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid of the individual, where the anti-Tau antibody is effective to reduce significantly the level of Tau (e.g., total Tau and/or free Tau) in an extracellular fluid within 48 hours, within 36 hours, within 24 hours, within 12 hours, within 8 hours, within 4 hours, within 2 hours, within 1 hour, or within 30 minutes (or less than 30 minutes) of administration of the anti-Tau antibody. For example, in some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid of the individual, where the anti-Tau antibody is effective to reduce significantly the level of Tau (e.g., total Tau and/or free Tau) in an extracellular fluid within a time period of from about 15 minutes to about 30 minutes, from about 30 minutes to about 1 hour, from about 1 hour to about 2 hours, from about 2 hours to about 4 hours, from about 4 hours to about 8 hours, from about 8 hours to about 12 hours, from about 12 hours to about 24 hours, from about 24 hours to about 36 hours, or from about 36 hours to about 48 hours.

In some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid (e.g., CSF, ISF, blood, or a blood fraction (e.g., serum or plasma)) of the individual, where the reduced level of Tau (e.g., total Tau and/or free Tau) is maintained for a period of time of at least 2 hours following administration of the anti-Tau antibody. For example, in some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid of the individual, where the reduced level of Tau (e.g., total Tau and/or free Tau) is maintained for a period of time of at least 2 hours, at least 4 hours, at least 8 hours, at least 12 hours, at least 24 hours, at least 36 hours, at least 48 hours, at least 72 hours, at least 96 hours, at least 120 hours, at least 144 hours, at least 168 hours, or more than 168 hours, following administration of the anti-Tau antibody. For example, in some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid of the individual, where the reduced level of Tau (e.g., total Tau and/or free Tau) is maintained for a period of time of from about 2 hours to about 4 hours, from about 4 hours to about 8 hours, from about 8 hours to about 12 hours, from about 12 hours to about 24 hours, from about 24 hours to about 36 hours, from about 36 hours to about 48 hours, from about 48 hours to about 72 hours, from about 72 hours to about 96 hours, from about 96 hours to about 120 hours, from about 120 hours to about 144 hours, from about 144 hours to about 168 hours, or more than 168 hours (e.g., 8 days, 10 days, 14 days, or longer than 14 days). In some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid of the individual, where the reduced level of Tau (e.g., total Tau and/or free Tau) is maintained for a period of time of at least 7 days, at least 10 days, at least 2 weeks, or at least 4 weeks. For example, in some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid of the individual, where the reduced level of Tau (e.g., total Tau and/or free Tau) is maintained for a period of time of from about 7 days to about 10 days, from about 10 days to about 2 weeks, or from about 2 weeks to about 4 weeks, or more than 4 weeks (e.g., 3 months, 4 months, 6 months, or more than 6 months).

In some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid (e.g., CSF, ISF, blood, or a blood fraction (e.g., serum or plasma)) of the individual, where the reduced level of Tau (e.g., total Tau and/or free Tau) is maintained for a period of time that provides for a reduction in the level of Aβ in the extracellular fluid (e.g., CSF, ISF, blood, or a blood fraction (e.g., serum or plasma)). For example, in some embodiments, the level of Abeta (Aβ) in the extracellular fluid is reduced significantly within a period of time of from about one day to about 25 days after administration of the anti-Tau Ab. For example, in some embodiments, the level of Aβ in the extracellular fluid is reduced significantly within a period of time of from about 1 day to about 5 days, from about 5 days to about 10 days, from about 10 days to about 15 days, from about 15 days to about 20 days, or from about 20 days to about 25 days, after administration of the anti-Tau Ab. The anti-Tau antibody can be administered to provide for continued suppression of Aβ levels over time. Aβ includes Aβ40 and Aβ42. In some cases, Aβ40 levels are reduced. In some cases, Aβ42 levels are reduced. In some cases, both Aβ42 and Aβ40 levels are reduced. A “significant reduction” in Aβ levels is an at least 5% reduction, an at least 10% reduction, an at least 15% reduction, an at least 20% reduction, an at least 25% reduction, an at least 30% reduction, an at least 40% reduction, an at least 45% reduction, an at least 50% reduction, or greater than 50% reduction, in the level of Aβ, compared to the level of Aβ in the absence of administration of the anti-Tau antibody (e.g., compared to the level of Aβ before administration of the anti-Tau antibody.

In some cases, a method of treating a tauopathy (e.g., an acute tauopathy) of the present disclosure involves administering to the individual an anti-Tau antibody in an amount effective to reduce significantly the level of Tau total Tau and/or free Tau) in an extracellular fluid (e.g., CSF, ISF, blood, or a blood fraction (e.g., serum or plasma)) of the individual, where the extracellular fluid is CSF. In some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid of the individual, where the extracellular fluid is ISF. In some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid of the individual, where the extracellular fluid is plasma.

In some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid (e.g., CSF, ISF, blood, or a blood fraction (e.g., serum or plasma)) of the individual, where anti-Tau antibody is administered by subcutaneous administration, e.g., by subcutaneous injection. In some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid of the individual, where anti-Tau antibody is administered by intrathecal administration. In some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid of the individual, where anti-Tau antibody is administered by intravenous administration, e.g., by intravenous injection.

In some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid (e.g., CSF, ISF, blood, or a blood fraction (e.g., serum or plasma)) of the individual, where anti-Tau antibody is administered in an amount of from about 0.1 mg/kg body weight to about 50 mg/kg body weight. For example, in some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid of the individual, where anti-Tau antibody is administered in an amount of from about 0.1 mg/kg body weight to about 0.5 mg/kg body weight, from about 0.5 mg/kg body weight to about 1 mg/kg body weight, from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, from about 40 mg/kg body weight to about 45 mg/kg body weight, or from about 45 mg/kg body weight to about 50 mg/kg body weight, or more than 50 mg/kg body weight.

In some cases, an anti-Tau antibody is administered in an amount of from about 0.1 mg/kg body weight to about 0.5 mg/kg body weight, from about 0.5 mg/kg body weight to about 1 mg/kg body weight, from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, from about 40 mg/kg body weight to about 45 mg/kg body weight, or from about 45 mg/kg body weight to about 50 mg/kg body weight, or more than 50 mg/kg body weight; and the anti-Tau antibody is administered in a single dose.

In some cases, an anti-Tau antibody is administered in an amount of from about 0.1 mg/kg body weight to about 0.5 mg/kg body weight, from about 0.5 mg/kg body weight to about 1 mg/kg body weight, from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, from about 40 mg/kg body weight to about 45 mg/kg body weight, or from about 45 mg/kg body weight to about 50 mg/kg body weight, or more than 50 mg/kg body weight; and the anti-Tau antibody is administered in multiple (2 or more) doses.

In some cases, an anti-Tau antibody is administered in an amount of from about 0.1 mg/kg body weight to about 0.5 mg/kg body weight, from about 0.5 mg/kg body weight to about 1 mg/kg body weight, from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, from about 40 mg/kg body weight to about 45 mg/kg body weight, or from about 45 mg/kg body weight to about 50 mg/kg body weight, or more than 50 mg/kg body weight; and the anti-Tau antibody is administered in multiple doses, e.g., the anti-Tau antibody is administered once every hour, once every 2 hours, once every 3 hours, once every 4 hours, once every 5 hours, once every 6 hours, once every 7 hours, once every 8 hours, once every 9 hours, once every 10 hours, once every 12 hours, once every 24 hours, once every 48 hours, once every 3 days, once every 4 days, once every 5 days, once every 6 days, once every 7 days, once every 2 weeks, once per month, once every 2 months, once every 4 months, once every 6 months, or once per year.

In some cases, an anti-Tau antibody is administered in an amount of from about 0.1 mg/kg body weight to about 0.5 mg/kg body weight, from about 0.5 mg/kg body weight to about 1 mg/kg body weight, from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, from about 40 mg/kg body weight to about 45 mg/kg body weight, or from about 45 mg/kg body weight to about 50 mg/kg body weight, or more than 50 mg/kg body weight; and the anti-Tau antibody is administered in multiple doses, e.g., an initial dose of the anti-Tau antibody is administered within 30 minutes, within 1 hour, within 2 hours, within 4 hours, within 8 hours, within 12 hours, within 24 hours, within 2 days, within 4 days, within 1 week, within 2 weeks, within 4 weeks, or within 2 months, of an insult associated with physical disturbance to a subject's brain and/or associated tissues of the central nervous system that leads to elevated Tau levels; and a subsequent dose of the anti-Tau antibody is administered at a time period of from about 1 hour to about 1 year or more (e.g., from about 1 hour to about 4 hours, from about 4 hours to about 8 hours, from about 8 hours to about 12 hours, from about 12 hours to about 24 hours, from about 24 hours to about 2 days, from about 2 days to about 4 days, from about 4 days to about 7 days, from about 1 week to about 2 weeks, from about 2 weeks to about 4 weeks, from about 4 weeks to about 2 months, from about 2 months to about 4 months, from about 4 months to about 6 months, from about 6 months to about 1 year, or more than 1 year), after administration of the initial dose of the anti-Tau antibody.

In some cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid (e.g., CSF, BF, blood, or a blood fraction (e.g., serum or plasma)) of the individual, where anti-Tau antibody is administered in a single bolus injection.

In other cases, a method of treating a tauopathy (e.g., an acute tauopathy) of the present disclosure involves administering to the individual an anti-Tau antibody in an amount effective to reduce significantly the level of Tau (e.g., total Tau and/or free Tau) in an extracellular fluid (e.g., CSF, ISF, blood, or a blood fraction (e.g., serum or plasma)) of the individual, where anti-Tau antibody is administered in multiple doses (e.g., 2, 3, 4, 5, or more doses). Where multiple doses are administered, the dosing interval can be every hour, every 2 hours, every 3 hours, every 4 hours, every 5 hours, every 6 hours, every 7 hours, every 8 hours, every 9 hours, every 10 hours, every 12 hours, every 24 hours, every 48 hours, every 3 days, every 4 days, every 5 days, every 6 days, every 7 days, etc.

The present disclosure provides method of treating a tauopathy (e.g., an acute tauopathy) in an individual, where the method involves administering to the individual an anti-Tau antibody in an amount effective to provide for a minimal concentration of the anti-Tau antibody of in cerebrospinal fluid (CSF) of the individual. In some cases, the minimal concentration of anti-Tau antibody in the CSF is achieved within 30 minutes of administration of the anti-Tau antibody. In some cases, the minimal concentration of anti-Tau antibody in the CSF is achieved within 1 hour of administration of the anti-Tau antibody. In some cases, the minimal concentration of anti-Thu antibody in the CSF is achieved within 48 hours, within 36 hours, within 24 hours, within 12 hours, within 8 hours, within 4 hours, within 2 hours, within 1 hour, or within 30 minutes (or less than 30 minutes) of administration of the anti-Tau antibody. In some cases, the minimal concentration of anti-Tau antibody in the CSF is achieved within a time period of from about 15 minutes to about 30 minutes, from about 30 minutes to about 1 hour, from about 1 hour to about 2 hours, from about 2 hours to about 4 hours, from about 4 hours to about 8 hours, from about 8 hours to about 12 hours, from about 12 hours to about 24 hours, from about 24 hours to about 36 hours, or from about 36 hours to about 48 hours.

In some cases, a method of the present disclosure for treating a tauopathy (e.g., an acute tauopathy) in an individual involves administering to the individual an anti-Tau antibody in an amount effective to provide for a minimal concentration of the anti-Tau antibody in CSF in the individual, where the minimal concentration of anti-Tau antibody in the CSF is at least 20 ng/ml. For example, in some cases, a method of the present disclosure for treating a tauopathy (e.g., an acute tauopathy) in an individual involves administering to the individual an anti-Tau antibody in an amount effective to provide for a minimal concentration of the anti-Tau antibody in CSF in the individual, where the minimal concentration of anti-Tau antibody in the CSF is at least 20 ng/ml, at least 25 ng/ml, at least 30 ng/ml, at least 40 ng/ml, at least 50 ng/ml, at least 60 ng/ml, at least 75 ng/ml at least 100 ng/ml, at least 125 ng/ml, at least 150 ng/ml, at least 175 ng/ml, at least 200 ng/ml, at least 250 ng/ml, at least 300 ng/ml, at least 350 ng/ml, at least 400 ng/ml, at least 450 ng/ml, at least 500 ng/ml, at least 550 ng/ml, at least 600 ng/ml, at least 650 ng/ml, at least 700 ng/ml, at least 750 ng/ml, or at least 800 ng/ml. For example, in some cases, a method of the present disclosure for treating a tauopathy (e.g., an acute tauopathy) in an individual involves administering to the individual an anti-Tau antibody in an amount effective to provide for a minimal concentration of the anti-Tau antibody in CSF in the individual, where the minimal concentration of anti-Tau antibody in the CSF is from about 20 ng/ml to about 30 ng/ml, from about 30 ng/ml to about 40 ng/ml, from about 40 ng/ml to about 50 ng/ml, from about 50 ng/ml to about 60 ng/ml, from about 60 ng/ml to about 75 ng/ml, from about 75 ng/ml to about 100 ng/ml, from about 100 ng/ml to about 150 ng/ml, from about 150 ng/ml to about 200 ng/ml, from about 200 ng/ml to about 250 ng/ml, from about 250 ng/ml to about 300 ng/ml, from about 300 ng/ml to about 350 ng/ml, from about 350 ng/ml to about 400 ng/ml, from about 400 ng/ml to about 450 ng/ml, from about 450 ng/ml to about 500 ng/ml, from about 500 ng/ml to about 550 ng/ml, from about 550 ng/ml to about 600 ng/ml, from about 600 ng/ml to about 700 ng/ml, from about 700 ng/ml to about 800 ng/ml, or more than 800 ng/ml.

In some cases, a method of the present disclosure for treating a tauopathy (e.g., an acute tauopathy) in an individual involves administering to the individual an anti-Tau antibody in an amount effective to provide for a minimal concentration of the anti-Tau antibody of in CSF in the individual, where the minimal concentration of anti-Tau antibody in the CSF provides for a molar ratio of the anti-Tau antibody to Tau in the CSF of at least 2:1. For example, in some cases, a method of the present disclosure for treating a tauopathy (e.g., an acute tauopathy) in an individual involves administering to the individual an anti-Tau antibody in an amount effective to provide for a minimal concentration of the anti-Tau antibody of in CSF in the individual, where the minimal concentration of anti-Tau antibody in the CSF provides for a molar ratio of the anti-Tau antibody to Tau in the CSF of at least 2:1, at least 2.5:1, at least 3:1, at least 3.5:1, at least 4:1, at least 4.5:1, at least 5:1, at least 6:1, at least 7:1, at least 8:1, at least 9:1, or at least 10:1.

In some cases, a method of the present disclosure for treating a tauopathy (e.g., an acute tauopathy) in an individual involves administering to the individual an anti-Tau antibody in an amount effective to provide for a minimal concentration of the anti-Tau antibody of in CSF in the individual, where the acute tauopathy is traumatic brain injury. In some cases, a method of the present disclosure for treating a tauopathy (e.g., an acute tauopathy) in an individual involves administering to the individual an anti-Tau antibody in an amount effective to provide for a minimal concentration of the anti-Tau antibody of in CSF in the individual, where the acute tauopathy is stroke.

The present disclosure provides methods for treating traumatic brain injury (TBI) in an individual, the methods generally involving administering to the individual an anti-Tau antibody in an amount effective to reduce significantly the level of Tau (e.g., total Tau and/or free Tau) in an extracellular fluid of the individual. In some cases, the antibody is administered within 48 hours of the traumatic brain injury. In some cases, the antibody is administered within 48 hours, within 36 hours, within 24 hours, within 12 hours, within 8 hours, within 4 hours, within 2 hours, within 1 hour, or within 30 minutes (or less than 30 minutes) of the TBI.

The present disclosure provides methods for treating stroke in an individual, the methods generally involving administering to the individual an anti-Tau antibody in an amount effective to reduce significantly the level of Tau (e.g., total Tau and/or free Tau) in an extracellular fluid of the individual. In some cases, the antibody is administered within 48 hours of the stroke. In some cases, the antibody is administered within 48 hours, within 36 hours, within 24 hours, within 12 hours, within 8 hours, within 4 hours, within 2 hours, within 1 hour, or within 30 minutes (or less than 30 minutes) of the stroke.

The amount of free Tau, e.g., free extracellular Tau (eTau), unbound to an anti-eTau antibody in the extracellular fluid can be determined as follows. The amount of free Tau can be determined by a method involving: a) contacting an immobilized antibody with a sample of extracellular fluid (e.g., CSF, ISF, serum, blood, or plasma) obtained from an individual, where the immobilized antibody competes for binding to eTau with the anti-eTau antibody administered to the individual, and where the contacting is under conditions suitable for binding of the unbound eTau to the immobilized antibody; and b) determining the amount of eTau bound to the immobilized antibody. The amount of eTau bound to the immobilized antibody is an indication of the amount of eTau unbound to the anti-Tau antibody in the sample. In some cases, the amount of eTau bound to the immobilized antibody is determined using a detectably labeled third antibody that does not compete with the immobilized antibody for binding to the eTau.

Antibodies

Anti-Tau antibodies (or VH/VL domains or CDRs derived therefrom) suitable for use in the invention can be generated using methods well known in the art. Alternatively, art recognized anti-Tau antibodies can be used. Antibodies that bind to the same epitope and/or compete with any of these art-recognized antibodies for binding to Tau also can be used.

An exemplary anti-Tau antibody is hu-IPN002 (also known as IPN007 and IPN002 Variant 2) comprising heavy and light chains having the sequences shown in SEQ ID NOs:37 and 41, respectively, or antigen binding fragments and variants thereof. hu-IPN002 is a humanized immunoglobulin (IgG4) monoclonal antibody that binds to extracellular Tau.

In other embodiments, the antibody comprises the heavy and light chain CDRs or variable regions of hu-IPN002. Accordingly, in one embodiment, the antibody comprises the CDR1, CDR2, and CDR3 domains of the VH region of hu-IPN002 having the sequence set forth in SEQ ID NO:37, and the CDR1, CDR2 and CDR3 domains of the VL region of hu-IPN002 having the sequence set forth in SEQ ID NO:41. In another embodiment, the antibody comprises heavy chain CDR1, CDR2 and CDR3 domains having the sequences set forth in SEQ ID NOs: 10, 11, and 12, respectively, and light chain CDR1, CDR2 and CDR3 domains having the sequences set forth in SEQ NOs:7, 8, and 9, respectively. In another embodiment, the antibody comprises VH and/or VL regions having the amino acid sequences set forth in SEQ ID NO:37 and/or SEQ ID NO: 41, respectively. In another embodiment, the antibody comprises the heavy chain variable (VH) and/or light chain variable (VL) regions encoded by the nucleic acid sequences set forth in SEQ ID NO:29 and/or SEQ ID NO:33, respectively. In another embodiment, the antibody competes for binding with, and/or binds to the same epitope on Tau as, the above-mentioned antibodies. In another embodiment, the antibody has at least about 90% variable region amino acid sequence identity with the above-mentioned antibodies (e.g., at least about 90%, 95% or 99% variable region identity with SEQ ID NO:37 or SEQ ID NO:41).

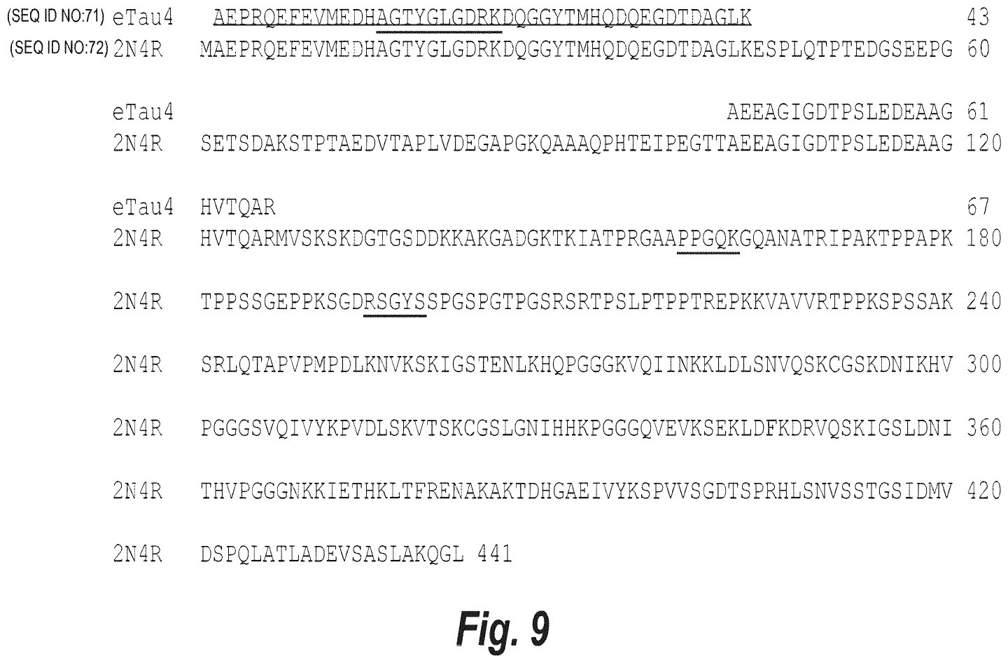

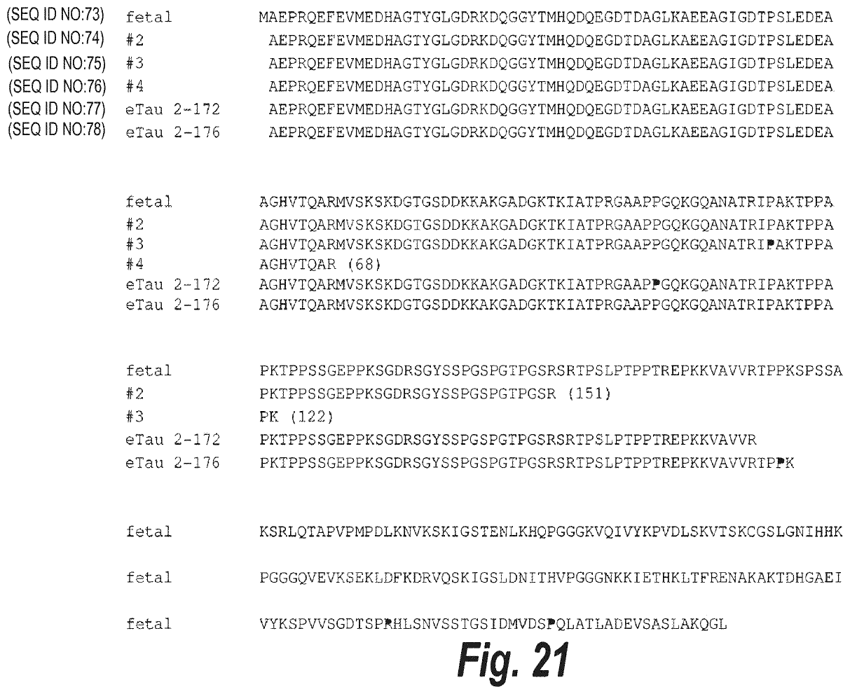

In one embodiment, an antibody that binds an N-terminal region of a Tau polypeptide, and that is suitable for use in a subject method of treating a tauopathy (e.g., an acute tauopathy), is an antibody that binds an epitope of Tau that is within amino acids 2-176 of Tau, e.g., within amino acids 2-15, amino acids 15-24, amino acids 24-50, amino acids 2-25, amino acids 15 to 50, amino acids 50 to 75, amino acids 40 to 60, amino acids 75 to 100, amino acids 60 to 80, amino acids 100 to 125, amino acids 80-115, amino acids 125 to 150, amino acids 115 to 140, amino acids 150 to 176, or amino acids 140 to 160, of Tau. Exemplary Tau polypeptides are depicted in FIG. 9; an antibody that is suitable for treating a tauopathy (e.g., an acute tauopathy) in an individual can be a humanized antibody that specifically binds an epitope in a Tau polypeptide depicted in FIG. 9. FIG. 21 depicts examples of eTau polypeptides; an antibody that is suitable for treating a tauopathy (e.g., an acute tauopathy) in an individual can be a humanized antibody that specifically binds an epitope in a Tau polypeptide depicted in FIG. 21.

A humanized antibody that binds an N-terminal region of a Tau polypeptide, and that is suitable for use in a subject method of treating a tauopathy (e.g., an acute tauopathy), is a humanized antibody that binds an epitope of Tau that is within amino acids 2-176 of Tau, e.g., within amino acids 2-15, amino acids 15-24, amino acids 24-50, amino acids 2-25, amino acids 15 to 50, amino acids 50 to 75, amino acids 40 to 60, amino acids 75 to 100, amino acids 60 to 80, amino acids 100 to 125, amino acids 80-115, amino acids 125 to 150, amino acids 115 to 140, amino acids 150 to 176, or amino acids 140 to 160, of Tau. Exemplary Tau polypeptides are depicted in FIG. 9; an antibody that binds an N-terminal region of a Tau polypeptide, and that is suitable for use in a subject method of treating a tauopathy, can be a humanized antibody that specifically binds an epitope in a Tau polypeptide depicted in FIG. 9.

In some cases, an antibody that that binds an N-terminal region of a Tau polypeptide, and that is suitable for use in a subject method of treating a tauopathy (e.g., an acute tauopathy), is a humanized anti-Tau antibody that binds an epitope within amino acids 15-24 of Tau.

In some cases, an antibody that binds an N-terminal region of a Tau polypeptide, and that is suitable for use in a subject method of treating a tauopathy (e.g., an acute tauopathy), is an antibody that binds an epitope of Tau that is within amino acids 1-158 of Tau, e.g., within amino acids 1-15, amino acids 7-13, amino acids 2-18, amino acids 15-24, amino acids 19-46, amino acids 24-50, amino acids 2-25, amino acids 25-30, amino acids 15 to 50, amino acids 28-126, amino acids 50 to 75, amino acids 40 to 60, amino acids 75 to 100, amino acids 60 to 80, amino acids 100 to 125, amino acids 80-115, amino acids 12.5 to 150, amino acids 115 to 140, or amino acids 150 to 158, of Tau, where the amino acid numbering is based on the amino acid number of 2N4R Tau, e.g., as depicted in FIG. 9. In some cases, the antibody is humanized.

In some cases, the methods of the present disclosure involve treating a tauopathy (e.g., an acute tauopathy) by administering an anti-Tau antibody, wherein the epitope bound by the antibody comprises amino acid residues within amino acids 1-158 of Tau, where the amino acid numbering is based on the 2N4R Tau amino acid sequence depicted in FIG. 9. In some cases, the anti-Tau antibody that is administered specifically binds Tau, where the epitope bound by the antibody comprises amino acid residues within amino acids 2-18 of Tau. In some cases, the anti-Tau antibody that is administered specifically binds Tau, where the epitope bound by the antibody is a linear epitope, and where the epitope bound by the antibody comprises amino acid residues within amino acids 2-68 of Tau. In some cases, the anti-Tau antibody that is administered specifically binds a Tau4 polypeptide having at least 95%, at least 98%, at least 99%, or 100%, amino acid sequence identity to the amino acid sequence set forth in SEQ ID NO: 71. In some cases, the anti-Tau antibody that is administered specifically binds a linear epitope within a Tau polypeptide, where the epitope is within amino acids 2-68 of Tau. In some cases, the anti-Tau antibody that is administered specifically binds a linear epitope within a Tau polypeptide, where the epitope is within amino acids 15-24 of Tau. In some cases, the anti-Tau antibody that is administered specifically binds Tau, where the epitope bound by the antibody comprises amino acid residues within amino acids 7-13 of Tau, e.g., amino acids EFEVMED (SEQ ID NO: 21). In some cases, the anti-Tau antibody that is administered specifically binds Tau, where the epitope bound by the antibody comprises amino acid residues within amino acids 25-30 of Tau, e.g., amino acids DQGGYT (SEQ ID NO: 22). In some cases, the anti-Tau antibody that is administered specifically binds Tau, where the epitope bound by the antibody comprises amino acid residues within amino acids 28-126 of Tau, where the amino acid numbering is based on the 2N4R Tau amino acid sequence depicted in FIG. 9. In some cases, the anti-Tau antibody that is administered specifically binds Tau, where the epitope bound by the antibody comprises amino acid residues within amino acids 150-158 of Tau, where the amino acid numbering is based on the 2N4R Tau amino acid sequence depicted in FIG. 9. In some cases, the anti-Tau antibody that is administered specifically binds Tau, where the epitope bound by the antibody comprises amino acid residues within amino acids 19-46 of Tau, where the amino acid numbering is based on the 2N4R Tau amino acid sequence depicted in FIG. 9.

In some cases, a method of the present disclosure involves treating a tauopathy (e.g., an acute tauopathy) by administering an antibody that specifically bind extracellular Tau (eTau), where the epitope bound by the antibody comprises amino acid residues within amino acids 1-158 of eTau, where the amino acid numbering is based on the 2N4R Tau amino acid sequence depicted in FIG. 9. In some cases, the anti-Tau antibody that is administered specifically binds eTau, where the epitope bound by the antibody comprises amino acid residues within amino acids 2-18 of eTau. In some cases, the anti-Tau antibody that is administered specifically binds eTau, where the epitope bound by the antibody is a linear epitope, and where the epitope bound by the antibody comprises amino acid residues within amino acids 2-68 of eTau. In some cases, the anti-Tau antibody that is administered specifically binds an eTau4 polypeptide having at least 95%, at least 98%, at least 99%, or 100%, amino acid sequence identity to the amino acid sequence set forth in SEQ ID NO: 71. In some cases, the anti-Tau antibody that is administered specifically binds a linear epitope within an eTau4 polypeptide, where the epitope is within amino acids 2-68 of eTau4. In some cases, the anti-Tau antibody that is administered specifically binds a linear epitope within an eTau4 polypeptide, where the epitope is within amino acids 15-24 of eTau4. In some cases, the anti-Tau antibody that is administered specifically binds eTau, where the epitope bound by the antibody comprises amino acid residues within amino acids 7-13 of eTau, e.g., amino acids EFEVMED (SEQ ID NO: 21). In some cases, the anti-Tau antibody that is administered specifically binds eTau, where the epitope bound by the antibody comprises amino acid residues within amino acids 25-30 of eTau, e.g., amino acids DQGGYT (SEQ ID NO: 22). In some cases, the anti-Tau antibody that is administered specifically binds eTau, where the epitope bound by the antibody comprises amino acid residues within amino acids 28-126 of eTau, where the amino acid numbering is based on the 2N4R Tau amino acid sequence depicted in FIG. 9. In some cases, the anti-Tau antibody that is administered specifically binds eTau, where the epitope bound by the antibody comprises amino acid residues within amino acids 150-158 of eTau, where the amino acid numbering is based on the 2N4R Tau amino acid sequence depicted in FIG. 9. In some cases, the anti-Tau antibody that is administered specifically binds eTau, where the epitope bound by the antibody comprises amino acid residues within amino acids 19-46 of eTau, where the amino acid numbering is based on the 2N4R Tau amino acid sequence depicted in FIG. 9.

The present disclosure provides a method of treating a tauopathy (e.g., an acute tauopathy) in an individual. The method generally involves administering to the individual: a) an effective amount of an antibody (e.g., a monoclonal antibody), which antibody may optionally be a humanized antibody, that binds an N-terminal region of a Tau polypeptide; or b) a pharmaceutical composition comprising the humanized antibody.

An antibody that binds an N-terminal region of a Tau polypeptide (optionally a humanized antibody, e.g., a monoclonal antibody) and that is suitable for use in a subject method of treating a tauopathy (e.g., an acute tauopathy), is an antibody that binds an epitope of Tau that is within amino acids 1-158 of Tau, e.g., within amino acids 1-15, amino acids 7-13, amino acids 2-18, amino acids 15-24, amino acids 19-46, amino acids 24-50, amino acids 2-25, amino acids 25-30, amino acids 15 to 50, amino acids 28-126, amino acids 50 to 75, amino acids 40 to 60, amino acids 75 to 100, amino acids 60 to 80, amino acids 100 to 125, amino acids 80-115, amino acids 125 to 150, amino acids 115 to 140, or amino acids 150 to 158, of Tau, where the amino acid numbering is based on the amino acid number of 2N4R Tau, e.g., as depicted in FIG. 9. In some cases, the antibody is humanized.

In some cases, an antibody that binds an N-terminal region of a Tau polypeptide, and that is suitable for use in a subject method of treating a tauopathy (e.g., an acute tauopathy), is a humanized anti-Tau antibody of the present disclosure. In some cases, the antibody is a humanized antibody that binds an epitope (e.g., a linear epitope) within amino acids 15-24 of Tau.

In some cases, the method of treating a tauopathy (e.g., an acute tauopathy) in an individual involves administering to the individual an effective amount of an anti-Tau antibody that does not require the presence of the 2N insert of Tau for binding to Tau. In some cases, the epitope recognized by an anti-Tau antibody suitable for use in a subject method of treating a tauopathy is not within the 2N insert of Tau. The 2N insert of Tau includes amino acids 45-102 of the 2N4R amino acid sequence depicted in FIG. 9.

In some cases, an anti-Tau antibody that binds an N-terminal region of a Tau polypeptide, and that is suitable for use in a subject method of treating a tauopathy (e.g., an acute tauopathy), specifically binds Tau, where the epitope bound by the antibody comprises amino acid residues within amino acids 2-68 of Tau. In some cases, an anti-Tau antibody that binds an N-terminal region of a Tau polypeptide, and that is suitable for use in a subject method of treating a tauopathy (e.g., an acute tauopathy), specifically binds extracellular Tau (eTau), where the epitope bound by the antibody comprises amino acid residues within amino acids 2-68 of eTau. In some cases, an anti-Tau antibody that binds an N-terminal region of a Tau polypeptide, and that is suitable for use in a subject method of treating a tauopathy (e.g., an acute tauopathy), specifically binds eTau, where the epitope bound by the antibody is a linear epitope, and where the epitope bound by the antibody comprises amino acid residues within amino acids 2-68 of eTau. In some cases, an anti-Tau antibody that binds an N-terminal region of a Tau polypeptide, and that is suitable for use in a subject method of treating a tauopathy (e.g., an acute tauopathy), specifically binds an eTau4 polypeptide having at least 95%, at least 98%, at least 99%, or 100%, amino acid sequence identity to the amino acid sequence set forth in SEQ ID NO:48. In some cases, an anti-Tau antibody that binds an N-terminal region of a Tau polypeptide, and that is suitable for use in a subject method of treating a tauopathy (e.g., an acute tauopathy), specifically binds a linear epitope within an eTau4 polypeptide, where the epitope is within amino acids 2-68 of eTau4. In any of the above-noted embodiments, the antibody can be humanized.

In some cases, an antibody that binds an N-terminal region of a Tau polypeptide, and that is suitable for use in a subject method of treating a tauopathy (e.g., an acute tauopathy), where the epitope bound by the antibody comprises amino acid residues within amino acids 1-158 of Tau, where the amino acid numbering is based on a 2N4R form of Tau, e.g., as depicted in FIG. 9. In some of these embodiments, the antibody is humanized. In some of these embodiments, the epitope is a linear epitope.

In some cases, an antibody that binds an N-terminal region of a Tau polypeptide, and that is suitable for use in a subject method of treating a tauopathy (e.g., an acute tauopathy), specifically binds Tau, where the epitope bound by the antibody comprises amino acid residues within amino acids 2-18 of Tau, where the amino acid numbering is based on a 2N4R form of Tau, e.g., as depicted in FIG. 9. In some of these embodiments, the antibody is humanized. In some of these embodiments, the epitope is a linear epitope.

In some cases, an antibody that binds an N-terminal region of a Tau polypeptide, and that is suitable for use in a subject method of treating a tauopathy (e.g., an acute tauopathy), specifically binds Tau, where the epitope bound by the antibody comprises amino acid residues within amino acids 7-13 of Tau, where the amino acid numbering is based on a 2N4R form of Tau, e.g., as depicted in FIG. 9. In some of these embodiments, the antibody is humanized. In some of these embodiments, the epitope is a linear epitope.

In some cases, an antibody that binds an N-terminal region of a Tau polypeptide, and that is suitable for use in a subject method of treating a tauopathy (e.g., an acute tauopathy), specifically binds Tau, where the epitope bound by the antibody comprises amino acid residues within amino acids 25-30 of Tau, where the amino acid numbering is based on a 2N4R form of Tau, e.g., as depicted in FIG. 9. In some of these embodiments, the antibody is humanized. In some of these embodiments, the epitope is a linear epitope.

In some cases, an antibody that binds an N-terminal region of a Tau polypeptide, and that is suitable for use in a subject method of treating a tauopathy (e.g., an acute tauopathy), specifically binds Tau, where the epitope bound by the antibody comprises amino acid residues within amino acids 28-126 of Tau, where the amino acid numbering is based on a 2N4R form of Tau, e.g., as depicted in FIG. 9, In some of these embodiments, the antibody is humanized. In some of these embodiments, the epitope is a linear epitope.

In some cases, an antibody that binds an N-terminal region of a Tau polypeptide, and that is suitable for use in a subject method of treating a tauopathy (e.g., an acute tauopathy), specifically binds Tau, where the epitope bound by the antibody comprises amino acid residues within amino acids 150-158 of Tau, where the amino acid numbering is based on a 2N4R form of Tau, e.g., as depicted in FIG. 9. In some of these embodiments, the antibody is humanized. In some of these embodiments, the epitope is a linear epitope.

In some cases, an anti-Tau antibody suitable for use in a method of the present disclosure is an anti-Tau antibody that specifically binds an epitope within an N-terminal region of a Tau polypeptide (e.g., a linear epitope within an amino-terminal (N-terminal) portion of Tau, e.g., within amino acids 1-25 of Tau, within amino acids 1-18 of Tau, within amino acids 9 to 18 of Tau (where amino acids 1-18 of Tau are: MAEPRQEFEVMEDHAGTY; SEQ ID NO: 23), within amino acids 15-44 of Tau, within amino acids 13-24 of Tau, or within amino acids 15-24 of Tau (where amino acids 15-24 of Tau are: AGTYGLGDRK (SEQ ID NO: 24). In some instances, the antibody is humanized, e.g., one or more framework regions of the heavy chain variable region and/or the light chain variable region includes sequences derived from a human immunoglobulin framework.

In some cases, a humanized monoclonal antibody that is suitable for use in a subject method specifically binds an epitope within amino acids 15-24 of a Tau polypeptide. In some cases, the epitope does not comprise a phosphorylated amino acid. In some case, the epitope does not comprise a nitrated amino acid. In some cases, the epitope comprises a phosphorylated amino acid, a nitrated amino acid, or both a phosphorylated amino acid and a nitrated amino acid.

In some cases, an antibody that is suitable for use in a subject method is humanized. Humanization of a framework region(s) reduces the risk of the antibody eliciting a human-anti-mouse-antibody (HAMA) response in humans. Art-recognized methods of determining immune response can be performed to monitor a HAMA response in a particular patient or during clinical trials. Patients administered humanized antibodies can be given an immunogenicity assessment at the beginning and throughout the administration of the therapy. The HAMA response is measured, for example, by detecting antibodies to the humanized therapeutic reagent, in serum samples from the patient using a method known to one in the art, including surface plasmon resonance technology (BIACORE) and/or solid-phase enzyme-linked immunosorbent assay (ELISA) analysis. In many cases, a suitable humanized anti-Tau antibody does not substantially elicit a HAMA response in a human subject. In some cases, a suitable humanized anti-Tau antibody has reduced immunogenic potential, as determined by an EpiScreen™ assay performed using CD8⁺-depleted peripheral blood mononuclear cells. In some cases, a suitable humanized anti-Tau antibody exhibits a Stimulation index of less than 2.0.

Certain amino acids from the human variable region framework residues are selected for substitution based on their possible influence on CDR conformation and/or binding to antigen. The unnatural juxtaposition of murine CDR regions with human variable framework region can result in unnatural conformational restraints, which, unless corrected by substitution of certain amino acid residues, lead to loss of binding affinity.

The selection of amino acid residues for substitution can be determined, in part, by computer modeling. Computer hardware and software for producing three-dimensional images of immunoglobulin molecules are known in the art. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, e.g., those sharing at least 60%, 70%, 80%, 90%, or more than 90%, sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

CDR and framework regions are as defined by Kabat, Sequences of Proteins of Immunological interest (National Institutes of Health, Bethesda, Md., 1987 and 1991) An alternative structural definition has been proposed by Chothia et al., J. Mol. Biol. 196:901 (1987); Nature 342:878 (1989); and. J. Mol. Biol. 186:651 (1989) (collectively referred to as “Chothia”). When framework residues, as defined by Kabat, supra, constitute structural loop residues as defined by Chothia, supra, the amino acids present in the mouse antibody may be selected for substitution into the humanized antibody. Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia. (See e.g., Chothia and Lesk JMB 196:901 (1987)). These amino acids are particularly likely to interact with the amino acids in the CDRs and, if chosen from the acceptor, to distort the donor CDRs and reduce affinity. Moreover, the adjacent amino acids may interact directly with the antigen (Amit et al., Science, 233:747 (1986)) and selecting these amino acids from the donor may be desirable to keep all the antigen contacts that provide affinity in the original antibody.

An antibody suitable for use in a subject method can comprise a humanized light chain framework region; and a humanized heavy chain framework region, wherein the isolated antibody competes for binding to an epitope in an N-terminal region of a Tau polypeptide with an antibody that comprises: a) a light chain region comprising: i) a VL CDR1 comprising an amino acid sequence of SEQ ID NO:1 or SEQ ID NO:7; (ii) a VL CDR2 comprising an amino acid sequence of SEQ NO:2 or SEQ ID NO:8; and (iii) a VL CDR3 comprising an amino acid sequence of SEQ ID NO:3 or SEQ ID NO:9; and b) a heavy chain region comprising: (i) a VH CDR1 comprising an amino acid sequence of SEQ ID NO:4 or SEQ ID NO:10; (ii) a VH CDR2 comprising an amino acid sequence of SEQ ID NO:5 or SEQ ID NO:11; and (iii) a VH CDR3 comprising an amino acid sequence of SEQ ID NO:6 or SEQ ID NO:12. In some cases, the light chain region and the heavy chain region are present in separate polypeptides. In other cases, the light chain region and the heavy chain region are present in a single polypeptide. The isolated antibody can include a heavy chain that comprises a constant region of the isotype IgG1, IgG2, IgG3, or IgG4. In other cases, the antibody is a Fv, scFv, Fab, F(ab′)2, or Fab′. The antibody can comprise a covalently linked non-peptide synthetic polymer, e.g., where the synthetic polymer is a polyethylene glycol) polymer. In some cases, the isolated antibody is fused, directly or via a linker, to a carrier molecule, a peptide or a protein that promotes the crossing of the blood-brain barrier. In some cases, the epitope bound by the isolated antibody is within amino acids 15-24 of a Tau polypeptide. The isolated antibody humanized light chain framework region can comprise 1, 2, 3, 4, 5, 6, 7, 8, 9, or 10 of the amino acid substitutions depicted in Table 3. The isolated antibody humanized heavy chain framework region comprises 1, 2, 3, 4, 5, 6, 7, 8, 9, 10, 11, or 12 of the amino acid substitutions depicted in Table 2.

In some embodiments, an antibody suitable for use in a subject method can comprise: a) a light chain region comprising: i) one, two, or three complementarity determining regions (CDRs) of an IPN001 antibody, where the CDRs are as defined by Kabat (see, e.g., Table 1, above; and Kabat et al., U.S. Dept. of Health and Human Services, “Sequences of proteins of immunological interest” (1991)).

In some embodiments, an antibody suitable for use in a subject method comprises: a) a light chain region comprising: i) one, two, or three VL CDRs of an IPN001 antibody; and ii) a humanized light chain framework region; and b) a heavy chain region comprising: i) one, two, or three VH CDRs of an IPN001 antibody; and a humanized heavy chain framework region; where the VH and VL CDRs are as defined by Kabat (see, e.g., Table 1, above; and Kabat et al., U.S. Dept. of Health and Human Services, “Sequences of proteins of immunological interest” (1991)). In some of these embodiments, the anti-Tau antibody includes a humanized VH and/or VL framework region.

In some embodiments, an antibody suitable for use in a subject method comprises: a) a light chain region comprising: i) one, two, or three VL CDRs of an IPN001 antibody; and ii) a humanized light chain framework region; and b) a heavy chain region comprising: i) one, two, or three VH CDRs of an IPN001 antibody; and ii) a humanized heavy chain framework region; where the VH and VL CDRs are as defined by Chothia (see, e.g., Table 1, above; and Chothia et al., J. Mol. Biol. 196:901-917 (1987)).

In some embodiments, an antibody suitable for use in a subject method comprises: a) a light chain region comprising: i) one, two, or three VL CDRs of an IPN002 antibody; and ii) a humanized light chain framework region; and b) a heavy chain region comprising: i) one, two, or three VH CDRs of an IPN002 antibody; and ii) a humanized heavy chain framework region; where the VH and VL CDRs are as defined by Kabat (see, e.g., Table 1, above; and Kabat et al., U.S. Dept. of Health and Human Services, “Sequences of proteins of immunological interest” (1991)).

In some embodiments, an antibody suitable for use in a subject method comprises: a) a light chain region comprising: i) one, two, or three VL CDRs of an IPN002 antibody; and ii) a humanized light chain framework region; and b) a heavy chain region comprising: i) one, two, or three VH CDRs of an IPN002 antibody; and ii) a humanized heavy chain framework region; where the VH and VL CDRs are as defined by Chothia (see, e.g., Table 1, above; and Chothia et al., J. Mol. Biol. 196:901-917 (1987)).

In some embodiments, an antibody suitable for use in a subject method comprises: a) a light chain region comprising: i) one, two, or three CDRs selected from SEQ ID NO:1, SEQ ID NO:2, and SEQ ID NO:3; and ii) a humanized light chain framework region; and b) a heavy chain region comprising: i) one, two, or three CDRs selected from SEQ NO:4, SEQ ID NO:5, and SEQ NO:6; and ii) a humanized heavy chain framework region.

In some embodiments, an antibody suitable for use in a subject method comprises: a) a light chain region comprising: i) one, two, or three CDRs selected from SEQ NO:7, SEQ ID NO:8, and SEQ ID NO:9; and ii) a humanized light chain framework region; and b) a heavy chain region comprising: i) one, two, or three CDRs selected from SEQ ID NO:10, SEQ ID NO:11, and SEQ ID NO:12; and ii) a humanized heavy chain framework region.

In some embodiments, an antibody suitable for use in a subject method comprises: a) a light chain region comprising: i) a VL CDR1 comprising an amino acid sequence of SEQ ID NO:1 or SEQ ID NO:7; (ii) a VL CDR2 comprising an amino acid sequence of SEQ ID NO:2 or SEQ ID NO:8; (iii) a VL CDR3 comprising an amino acid sequence of SEQ ID NO:3 or SEQ ID NO:9; and (iv) a humanized light chain framework region; and b) a heavy chain region comprising: (i) a VH CDR1 comprising an amino acid sequence of SEQ ID NO:4 or SEQ ID NO:10; (ii) a VH CDR2 comprising an amino acid sequence of SEQ ID NO:5 or SEQ ID NO:11; (iii) a VH CDR3 comprising an amino acid sequence of SEQ ID NO:6 or SEQ ID NO:12; and iv) a humanized heavy chain framework region.

In some embodiments, an antibody suitable for use in a subject method comprises a heavy chain variable region comprising one, two, or three of the heavy chain CDRs having an amino acid sequence selected from one or more of SEQ ID NOs:4, 5, and 6; and one, two, three, or four FR regions that are humanized. For example, in some embodiments, a suitable antibody comprises a heavy chain variable region that comprises, in order from N-terminus to C-terminus: a humanized heavy chain FR1; a CDR1 comprising the amino acid sequence set forth in SEQ ID NO:4; a humanized heavy chain FR2; a CDR2 comprising the amino acid sequence set forth in SEQ ID NO:5; a humanized heavy chain FR3; a CDR3 comprising the amino acid sequence set forth in SEQ ID NO:6; and a humanized heavy chain FR4.

In some embodiments, an antibody suitable for use in a subject method comprises one, two, or three of the light chain CDRs having a polypeptide sequence selected from one or more of SEQ ID NOs:1, 2, and 3; and one, two, three, or four FR regions that are humanized. For example, in some embodiments, a suitable antibody comprises a light chain variable region that comprises, in order from N-terminus to C-terminus: a humanized light chain FR1; a CDR1 comprising the amino acid sequence set forth in SEQ ID NO:1; a humanized light chain FR2; a CDR2 comprising the amino acid sequence set forth in SEQ ID NO:2; a humanized light chain FR3; a CDR3 comprising the amino acid sequence set forth in SEQ ID NO:3; and a humanized light chain FR4.

In some embodiments, an antibody suitable for use in a subject method comprises one, two, or three of the heavy chain CDRs having an amino acid sequence selected from one or more of SEQ ID NOs:10, 11, and 12; and one, two, three, or four FR regions that are humanized. For example, in some embodiments, a suitable antibody comprises a heavy chain variable region that comprises, in order from N-terminus to C-terminus: a humanized heavy chain FR1; a CDR1 comprising the amino acid sequence set forth in SEQ ID NO:10; a humanized heavy chain FR2; a CDR2 comprising the amino acid sequence set forth in SEQ ID NO:11; a humanized heavy chain FR3; a CDR3 comprising the amino acid sequence set forth in SEQ ID NO:12; and a humanized heavy chain FR4.

In some embodiments, an antibody suitable for use in a subject method comprises one, two, or three of the light chain CDRs having a polypeptide sequence selected from one or more of SEQ ID NOs:7, 8, and 9; and one, two, three, or four FR regions that are humanized. For example, in some embodiments, a suitable antibody comprises a light chain variable region that comprises, in order from N-terminus to C-terminus: a humanized light chain FR1; a CDR1 comprising the amino acid sequence set forth in SEQ ID NO:7; a humanized light chain FR2; a CDR2 comprising the amino acid sequence set forth in SEQ ID NO:8; a humanized light chain FR3; a CDR3 comprising the amino acid sequence set forth in SEQ ID NO:9; and a humanized light chain FR4.

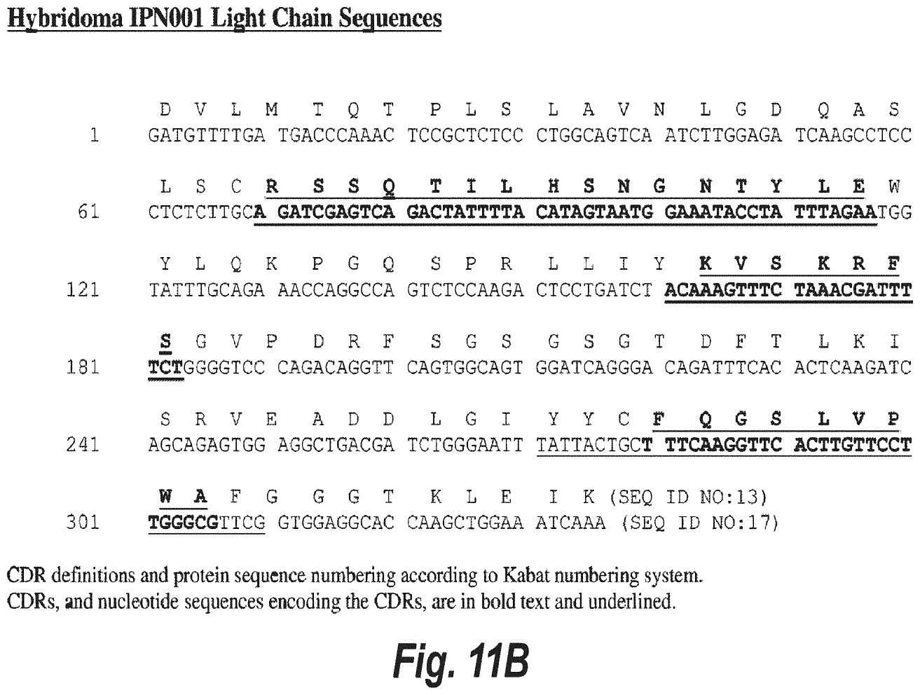

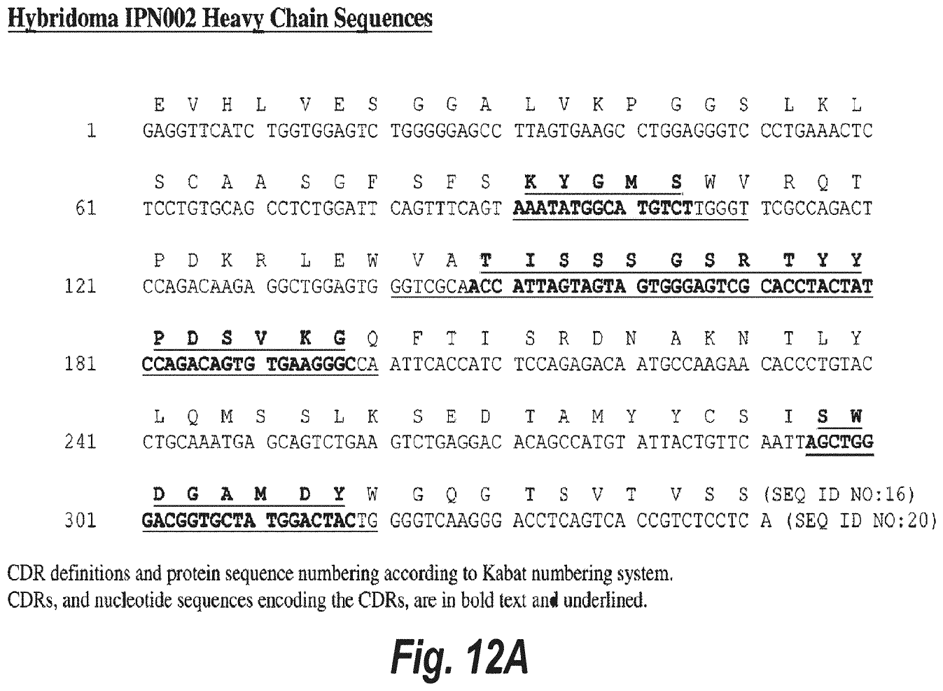

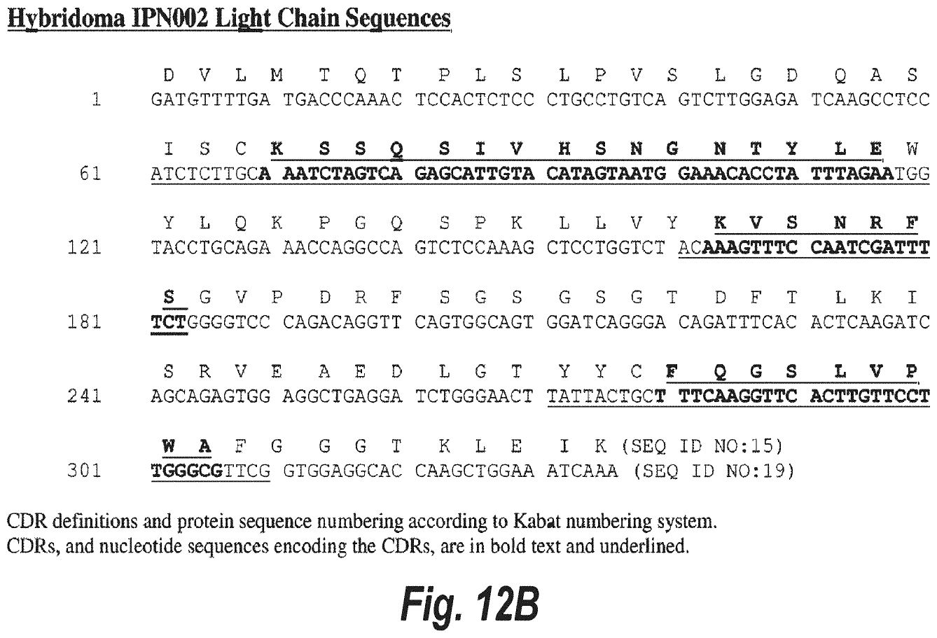

VH and VL amino acid sequences of IPN001 are depicted in FIGS. 11A and 11B. CDRs (as defined by Kabat) are in bold text and underlined. VH and VL amino acid sequences of IPN002 are depicted in FIGS. 12A and 12B. CDRs (as defined by Kabat) are in bold text and underlined.

SEQ ID NOs:1-12 are as follows:

(SEQ ID NO: 1)
RSSQTILHSNGNTYLE;
(SEQ ID NO: 2)
KVSKRFS;
(SEQ ID NO: 3)
FQGSLVPWA;
(SEQ ID NO: 4)
SYGMS;
(SEQ ID NO: 5)
TISSSGSRTYFPDSVKG;
(SEQ ID NO: 6)
TWDGAMDY;
(SEQ ID NO: 7)
KSSQSIVHSNGNTYLE;
(SEQ ID NO: 8)
KVSNRFS;
(SEQ ID NO: 9)
FQGSLVPWA;
(SEQ ID NO: 10)
KYGMS;
(SEQ ID NO: 11)
TISSSGSRTYYPDSVKG;
(SEQ ID NO: 12)
SWDGAMDY.

In some embodiments, an antibody suitable for use in a subject method can comprise a light chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 11B and set forth in SEQ ID NO:13.

In some embodiments, an antibody suitable for use in a subject method can comprise a heavy chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 11A and set forth in SEQ ID NO:14.

In some embodiments, an antibody suitable for use in a subject method can comprise a light chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 12B and set forth in SEQ ID NO:15.

In some embodiments, an antibody suitable for use in a subject method can comprise a heavy chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 12A and set forth in SEQ ID NO:16.

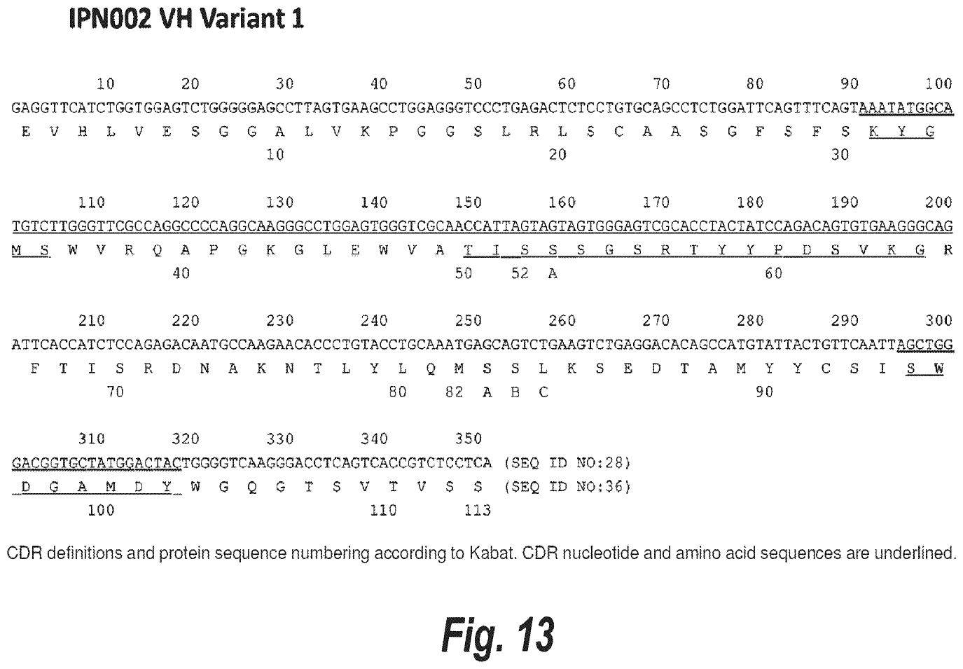

In some embodiments, an antibody suitable for use in a subject method can comprise a heavy chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 13 (VH variant 1).

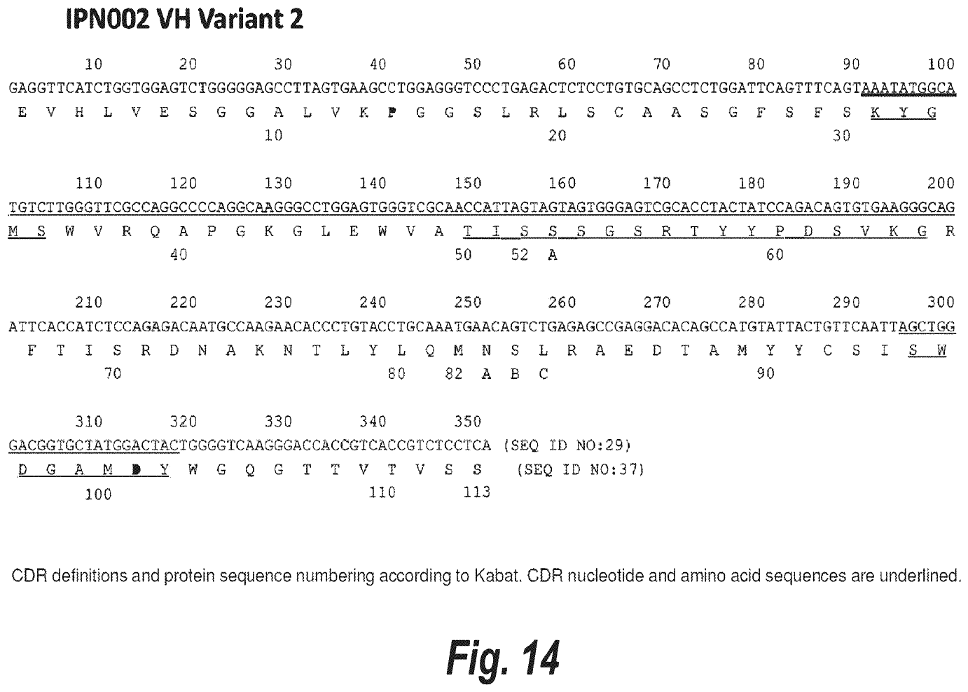

In some embodiments, an antibody suitable for use in a subject method can comprise a heavy chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 14 (VH variant 2).

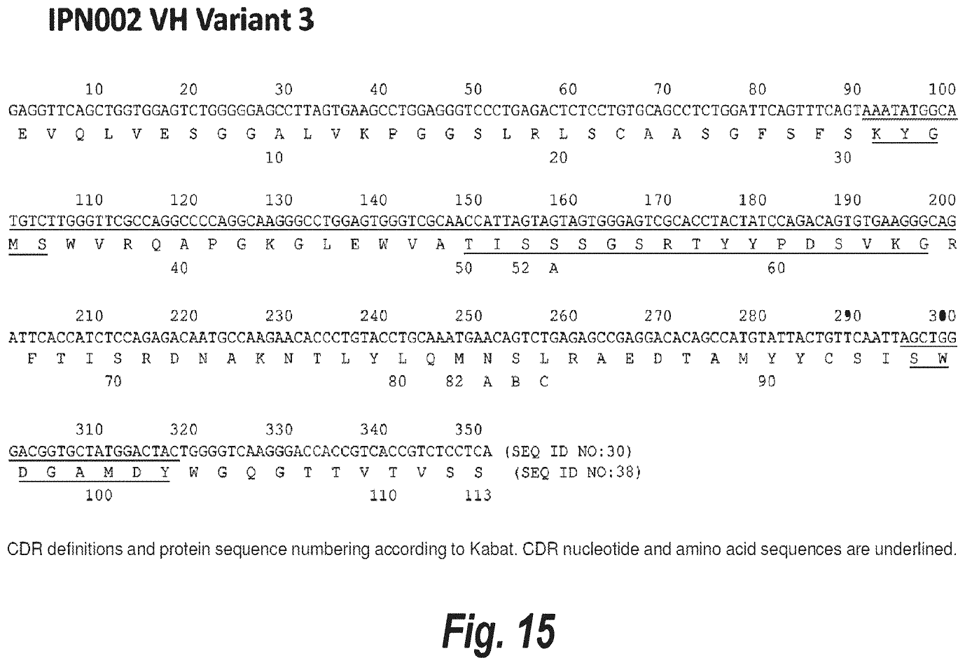

In some embodiments, an antibody suitable for use in a subject method can comprise a heavy chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 15 (VH variant 3).

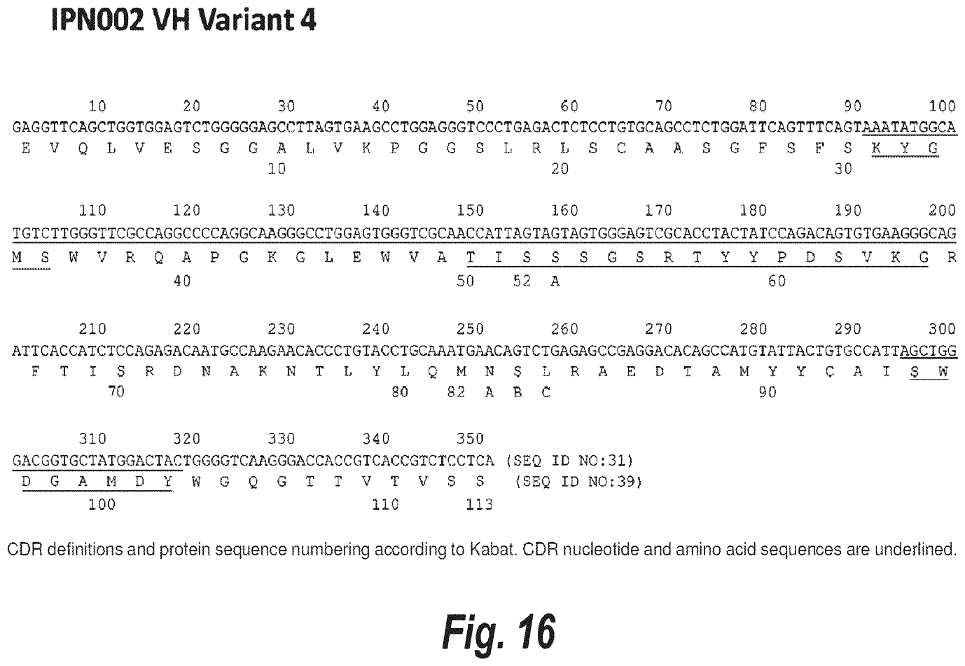

In some embodiments, an antibody suitable for use in a subject method can comprise a heavy chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 16 (VH variant 4).

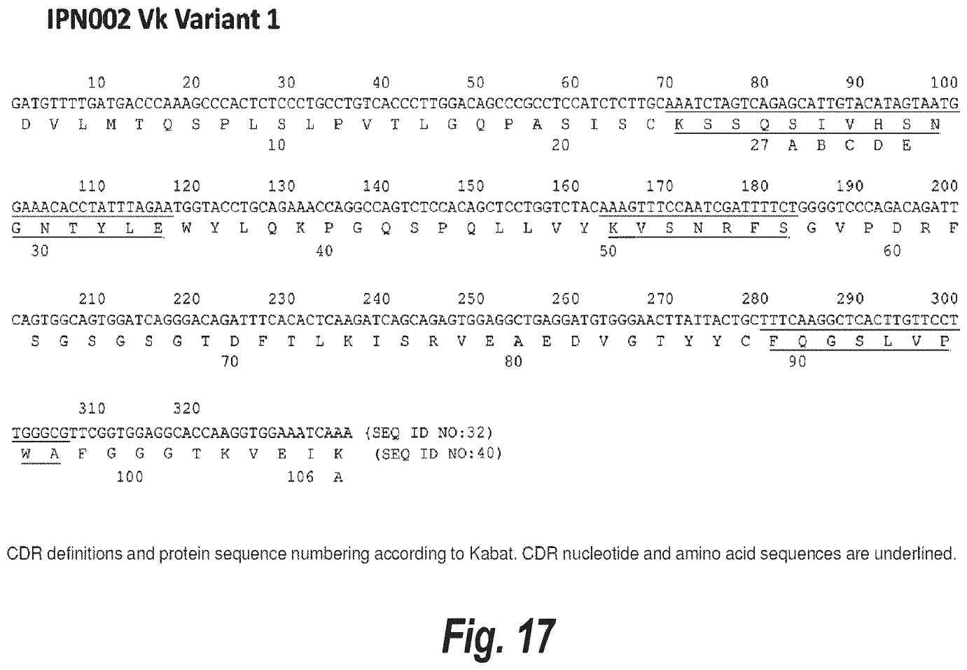

In some embodiments, an antibody suitable for use in a subject method can comprise a light chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 17 (Vk variant 1).

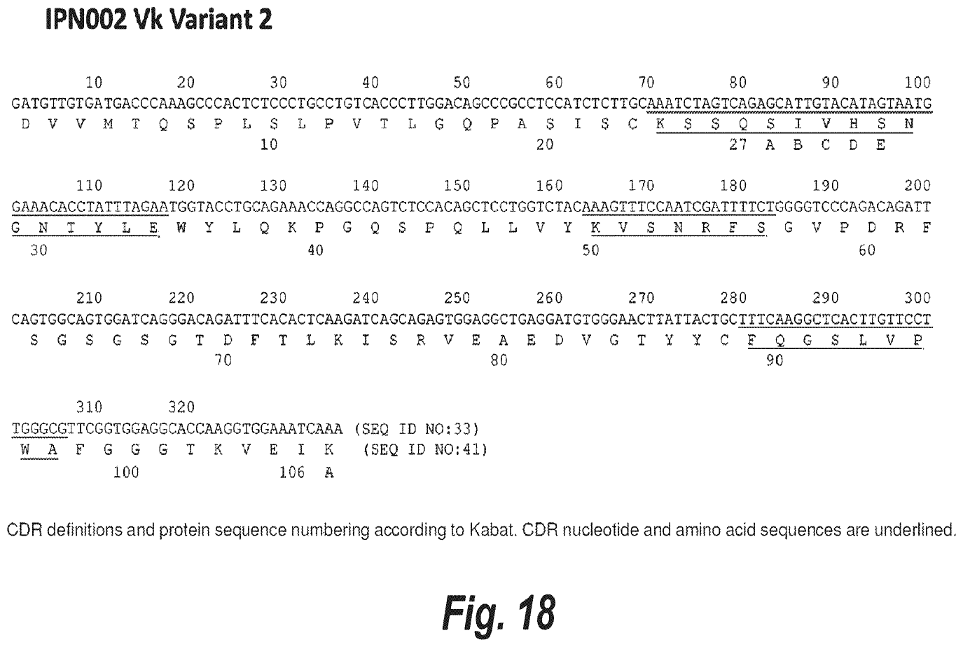

In some embodiments, an antibody suitable for use in a subject method can comprise a light chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 18 (Vk variant 2).

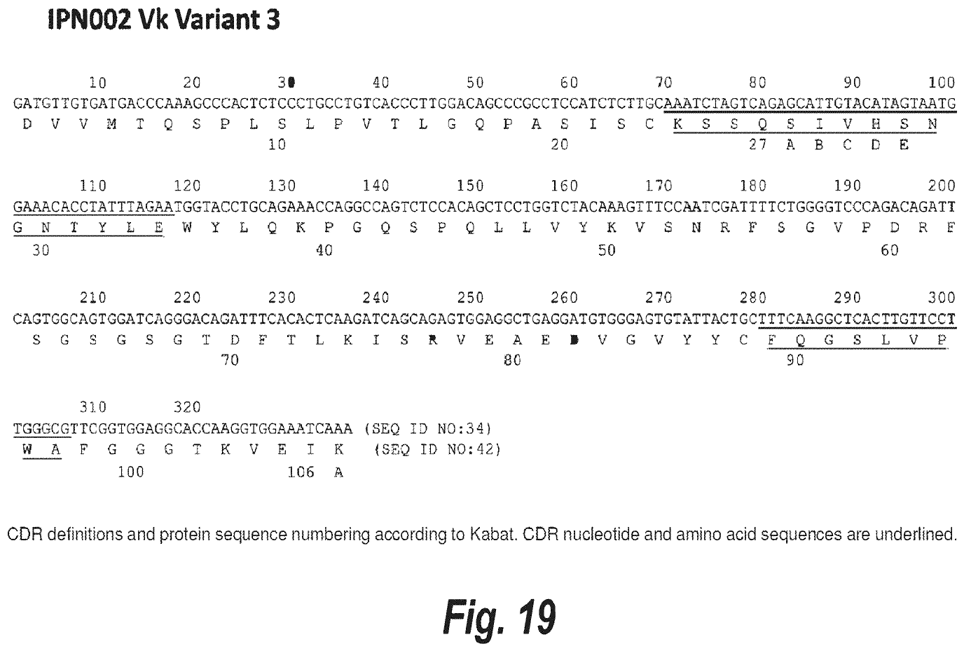

In some embodiments, an antibody suitable for use in a subject method can comprise a light chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 19 (Vk variant 3).

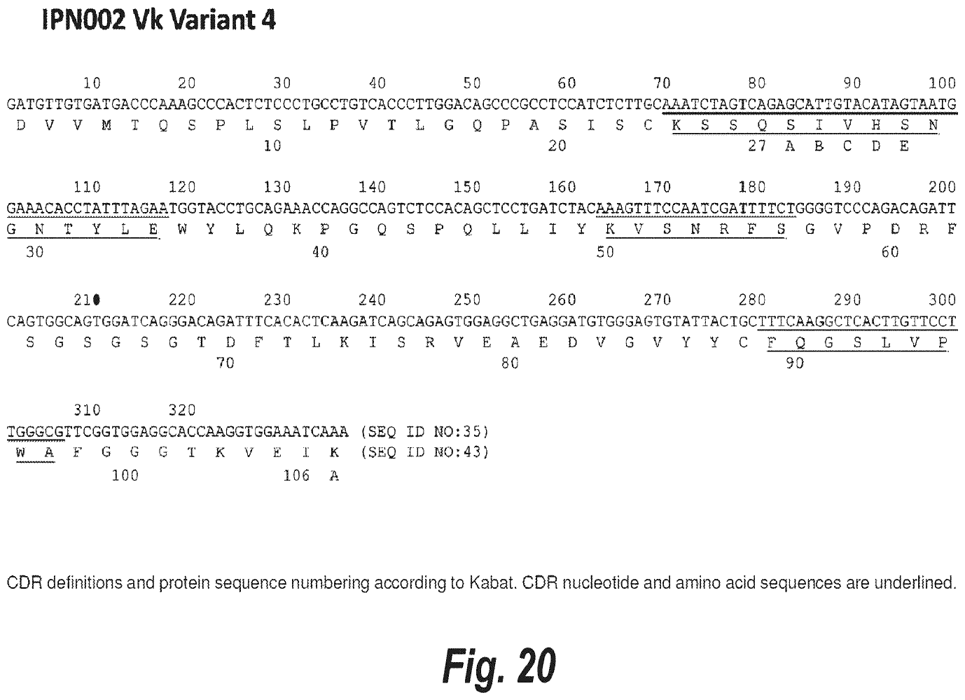

In some embodiments, an antibody suitable for use in a subject method can comprise a light chain variable region comprising an amino acid sequence that is 85%, 86%, 87%, 88%, 89%, 90%, 91%, 92%, 93%, 94%, 95%, 96%, 97%, 98% or 99% identical to the sequence depicted in FIG. 20 (Vk variant 4).

In some embodiments, an antibody suitable for use in a subject method can comprise a heavy chain variable region comprising 1, 2, 3, 4, 5, 6, 7, 8, 9, 10, 11, or 12 of the framework (FR) amino acid substitutions, relative to the IPN002 parental antibody FR amino acid sequences, depicted in Table 2.

TABLE 2
VH Variants

| Amino | IPN002 | | | | |
|---|---|---|---|---|---|
| Acid | (Parental | VH | VH | VH | |
| Position | antibody) | Variant 1 | Variant 2 | Variant 3 | VH Variant 4 |
| FR1 | | | | | |
| 3 | H | H | H | Q | Q |
| 19 | K | R | R | R | R |
| FR2 | | | | | |
| 40 | T | A | A | A | A |
| 42 | D | G | G | G | G |
| 44 | R | G | G | G | G |
| FR3 | | | | | |
| 66 | Q | R | R | R | R |
| 83 | S | S | N | N | N |
| 85 | L | S | L | L | L |
| 86 | K | K | R | R | R |
| 87 | S | S | A | A | A |
| 93 | S | S | S | S | A |
| FR4 | | | | | |
| 108 | S | S | T | T | T |

In some embodiments, an antibody suitable for use in a subject method can comprise a heavy chain variable region comprising an H→Q substitution at amino acid position 3 in VH FR1 and/or a K→R substitution at amino acid position 19 in VH FR1.

In some embodiments, an antibody suitable for use in a subject method can comprise a heavy chain variable region comprising a T→A substitution at amino acid position 40 in VH FR2 and/or a D→G substitution at amino acid position 42 in VH FR2 and/or an R→G substitution at position 44 in VH FR2.

In some embodiments, an antibody suitable for use in a subject method can comprise a heavy chain variable region comprising a Q→R substitution at amino acid position 66 in VH FR3 and/or an S→N substitution at amino acid position 83 in VH FR3 and/or an L→S substitution at amino acid position 85 in VH FR3 and/or a K→R substitution at amino acid position 86 in FR3 and/or an S→A substitution at amino acid position 87 in VH FR3 and/or an S→A substitution at amino acid position 93 in VH FR3.

In some embodiments, an antibody suitable for use in a subject method can comprise a heavy chain variable region comprising an S→T substitution at amino acid position 108 in VH FR4.

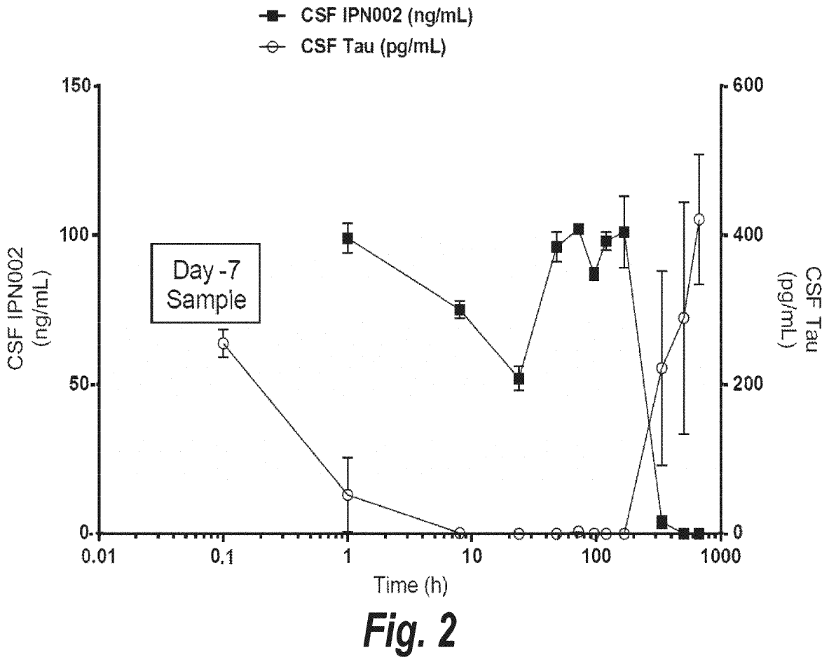

In some embodiments, an antibody suitable for use in a subject method can comprise, in order from N-terminus to C-terminus a VH region comprising: EVX1LVESGGALVKPGGSLRLSCAASGFSFS (SEQ ID NO: 25); VH CDR1 as shown in FIG. 2A; WVRQAPGKGLEWVA (SEQ ID NO: 26); VH CDR2 as shown in FIG. 2A; RFTISRDNAKNTLYLQMX2SX3X4X5EDTAMYYCX6I (SEQ ID NO: 27); VH CDR3 as shown in FIG. 2A; WGQGTX7VTVSS (SEQ ID NO: 44), where X1 is H or Q; X2 is S or N; X3 is S or L; X4 is K or R; X5 is S or A; X6 is S or A; and X7 is S or T.

In some embodiments, an antibody suitable for use in a subject method can comprise a light chain variable region comprising 1, 2, 3, 4, 5, 6, 7, 8, 9, or 10 of the framework (FR) amino acid substitutions, relative to the IPN002 parental antibody FR amino acid sequences, depicted in Table 3.

TABLE 3
Vk Variants

| | IPN002 | | | | |
|---|---|---|---|---|---|
| Amino Acid | (Parental | Vk | Vk | Vk | Vk |
| Position | antibody) | Variant 1 | Variant 2 | Variant 3 | Variant 4 |
| FR1 | | | | | |
| 3 | L | L | V | V | V |
| 7 | T | S | S | S | S |
| 14 | S | T | T | T | T |
| 17 | D | Q | Q | Q | Q |
| 18 | Q | P | P | P | P |
| FR2 | | | | | |
| 45 | K | Q | Q | Q | Q |
| 48 | V | V | V | V | I |
| FR3 | | | | | |
| 83 | L | V | V | V | V |
| 85 | T | T | T | V | V |
| FR4 | | | | | |
| 104 | L | V | V | V | V |

In some embodiments, an antibody suitable for use in a subject method can comprise a light chain variable region comprising an L→V substitution at amino acid position 3 in VL FR1 and/or a T→S substitution at amino acid position 7 in VL FR1 and/or an S→T substitution at amino acid position 14 in VL FR1 and/or a D→Q substitution at amino acid position 17 in VL FR1 and/or a Q→P substitution at amino acid position 18 in VL FR1.

In some embodiments, an antibody suitable for use in a subject method can comprise a light chain variable region comprising a substitution at amino acid position 45 of VL FR2 and/or a V→I substitution at amino acid position 48 of VL FR2.

In some embodiments, an antibody suitable for use in a subject method can comprise a light chain variable region comprising an L→V substitution at amino acid position 83 of VL FR3 and/or a T→V substitution at amino acid position 85 of VL FR3.

In some embodiments, an antibody suitable for use in a subject method can comprise a light chain variable region comprising an L→V substitution at amino acid position 104 of VL FR4.

In some embodiments, an antibody suitable for use in a subject method can comprise, in order from N-terminus to C-terminus a VL region comprising: DVX1MTQSPLSLPVTLGQPASISC (SEQ ID NO: 45); VL CDR1 as shown in FIG. 12B; WYLQKPGQSPQLLX2Y (SEQ ID NO: 46); VL CDR2 as shown in FIG. 12B; GVPDRFSGSGSGTDFTLKISRVEAEDVGX3YYC (SEQ ID NO: 47); VL CDR3 as shown in FIG. 2B; FGGGTKVEIK (SEQ ID NO: 48); where X1 is L or V; X2 is V or I; and X3 is T or V.

In some embodiments, an antibody suitable for use in a subject method comprises:
a) a VH variant 1 comprising the amino acid sequence depicted in FIG. 13; and a Vk variant 1 comprising the amino acid sequence depicted in FIG. 17;
b) a VH variant 1 comprising the amino acid sequence depicted in FIG. 13; and a Vk variant 2 comprising the amino acid sequence depicted in FIG. 18;
c) a VH variant 1 comprising the amino acid sequence depicted in FIG. 13; and a Vk variant 3 comprising the amino acid sequence depicted in FIG. 19;
d) a VH variant 1 comprising the amino acid sequence depicted in FIG. 13; and a Vk variant 4 comprising the amino acid sequence depicted in FIG. 20;
e) a VH variant 2 comprising the amino acid sequence depicted in FIG. 14; and a Vk variant 1 comprising the amino acid sequence depicted in FIG. 17;
f) a VH variant 2 comprising the amino acid sequence depicted in FIG. 14; and a Vk variant 2 comprising the amino acid sequence depicted in FIG. 18;
g) a VH variant 2 comprising the amino acid sequence depicted in FIG. 14; and a Vk variant 3 comprising the amino acid sequence depicted in FIG. 19;
h) a VH variant 2 comprising the amino acid sequence depicted in FIG. 14; and a Vk variant 4 comprising the amino acid sequence depicted in FIG. 20;
i) a VH variant 3 comprising the amino acid sequence depicted in FIG. 15; and a Vk variant 1 comprising the amino acid sequence depicted in FIG. 17;
j) a VH variant 3 comprising the amino acid sequence depicted in FIG. 15; and a Vk van ant 2 comprising the amino acid sequence depicted in FIG. 18;

k) a VH variant 3 comprising the amino acid sequence depicted in FIG. 15; and a Vk variant 3 comprising the amino acid sequence depicted in FIG. 19;

l) a VH variant 3 comprising the amino acid sequence depicted in FIG. 15; and a Vk variant 4 comprising the amino acid sequence depicted in FIG. 20;
m) a VH variant 4 comprising the amino acid sequence depicted in FIG. 16; and a Vk variant 1 comprising the amino acid sequence depicted in FIG. 17;
n) a VH variant 4 comprising the amino acid sequence depicted in FIG. 16; and a Vk variant 2 comprising the amino acid sequence depicted in FIG. 18;
o) a VH variant 4 comprising the amino acid sequence depicted in FIG. 16; and a Vk variant 3 comprising the amino acid sequence depicted in FIG. 19; or
p) a VH variant 4 comprising the amino acid sequence depicted in FIG. 16; and a Vk variant 4 comprising the amino acid sequence depicted in FIG. 20.

In some embodiments, an antibody suitable for use in a subject method comprises anti-Tau heavy chain CDRs and anti-Tau light chain CDRs in a single polypeptide chain, e.g., in some embodiments, a suitable antibody is a scFv. In some embodiments, a suitable antibody comprises, in order from N-terminus to C-terminus: a first amino acid sequence of from about 5 amino acids to about 25 amino acids in length; a CDR1 comprising the amino acid sequence set forth in SEQ ID NO:1; a second amino acid sequence of from about 5 amino acids to about 25 amino acids in length; a CDR2 comprising the amino acid sequence set forth in SEQ ID NO:2; a third amino acid sequence of from about 5 amino acids to about 25 amino acids in length; a CDR3 comprising the amino acid sequence set forth in SEQ ID NO:3; a fourth amino acid sequence of from about 5 amino acids to about 25 amino acids in length; a CDR1 comprising the amino acid sequence set forth in SEQ ID NO:4; a fifth amino acid sequence of from about 5 amino acids to about 25 amino acids in length; a CDR2 comprising the amino acid sequence set forth in SEQ NO:5; a sixth amino acid sequence of from about 5 amino acids to about 25 amino acids in length; a CDR3 comprising the amino acid sequence set forth in SEQ ID NO:6; and a seventh amino acid sequence of from about 5 amino acids to about 25 amino acids in length.

In some embodiments, an antibody suitable for use in a subject method comprises, in order from N-terminus to C-terminus: a light chain FR1 region; a CDR1 comprising the amino acid sequence set forth in SEQ ID NO:1; a light chain FR2 region; a CDR2 comprising the amino acid sequence set forth in SEQ ID NO:2; a light chain FR3 region; a CDR3 comprising the amino acid sequence set forth in SEQ ID NO:3; optionally a light chain FR4 region; a linker region; optionally a heavy chain FR1 region; a CDR1 comprising the amino acid sequence set forth in SEQ ID:4; a heavy chain FR2 region; a CDR2 comprising the amino acid sequence set forth in SEQ ID NO:5; a heavy chain FR3 region; a CDR3 comprising the amino acid sequence set forth in SEQ ID NO:6; and a heavy chain FR4 region. In some of these embodiments, one or more of the FR regions is a humanized FR region. In some of these embodiments, each of the FR regions is a humanized FR region. The linker region can be from about 5 amino acids to about 50 amino acids in length, e.g., from about 5 aa to about 10 aa, from about 10 aa to about 15 aa, from about 15 aa to about 20 aa, from about 20 aa to about 25 aa, from about 25 aa to about 30 aa, from about 30 aa to about 35 aa, from about 35 aa to about 40 aa, from about 40 aa to about 45 aa or from about 45 aa to about 50 aa in length.

In some embodiments, an antibody suitable for use in a subject method comprises, in order from N-terminus to C-terminus: a heavy chain FR1 region; a CDR1 comprising the amino acid sequence set forth in SEQ ID:4; a heavy chain FR2 region; a CDR2 comprising the amino acid sequence set forth in SEQ ID NO:5; a heavy chain FR3 region; a CDR3 comprising the amino acid sequence set forth in SEQ ID NO:6; optionally a heavy chain FR4 region; a linker; optionally a light chain FR1 region; a CDR1 comprising the amino acid sequence set forth in SEQ ID NO:1; a light chain FR2 region; a CDR2 comprising the amino acid sequence set forth in SEQ ID NO:2; a light chain FR3 region; a CDR3 comprising the amino acid sequence set forth in SEQ NO:3; and a light chain FR4 region. In some of these embodiments, one or more of the FR regions is a humanized FR region. In some of these embodiments, each of the FR regions is a humanized FR region. The linker region can be from about 5 amino acids to about 50 amino acids in length, e.g., from about 5 aa to about 10 aa, from about 10 aa to about 15 aa, from about 15 aa to about 20 aa, from about 20 aa to about 25 aa, from about 25 aa to about 30 aa, from about 30 aa to about 35 aa, from about 35 aa to about 40 aa, from about 40 aa to about 45 aa, or from about 45 aa to about 50 aa in length.

In some embodiments, an antibody suitable for use in a subject method comprises, in order from N-terminus to C-terminus: a light chain FR1 region; a CDR1 comprising the amino acid sequence set forth in SEQ ID NO:7; a light chain FR2 region; a CDR2 comprising the amino acid sequence set forth in SEQ ID NO:8; a light chain FR3 region; a CDR3 comprising the amino acid sequence set forth in SEQ ID NO:9; optionally a light chain FR4 region; a linker region; optionally a heavy chain FR1 region; a CDR1 comprising the amino acid sequence set forth in SEQ ID:10; a heavy chain FR2 region; a CDR2 comprising the amino acid sequence set forth in SEQ ID NO:11; a heavy chain FR3 region; a CDR3 comprising the amino acid sequence set forth in SEQ ID NO:12; and a heavy chain FR4 region. In some of these embodiments, one or more of the FR regions is a humanized FR region. In some of these embodiments, each of the FR regions is a humanized FR region. The linker region can be from about 5 amino acids to about 50 amino acids in length, e.g., from about 5 aa to about 10 aa, from about 10 aa to about 15 aa, from about 15 aa to about 20 aa, from about 20 aa to about 25 aa, from about 25 aa to about 30 aa, from about 30 aa to about 35 aa, from about 35 aa to about 40 aa, from about 40 aa to about 45 aa, or from about 45 aa to about 50 aa in length.

In some embodiments, an antibody suitable for use in a subject method comprises, in order from N-terminus to C-terminus: a heavy chain FR1 region; a CDR1 comprising the amino acid sequence set forth in SEQ ID:10; a heavy chain FR2 region; a CDR2 comprising the amino acid sequence set forth in SEQ ID NO:11; a heavy chain FR3 region; a CDR3 comprising the amino acid sequence set forth in SEQ ID NO:12; optionally a heavy chain FR4 region; a linker; optionally a light chain FR1 region; a CDR1 comprising the amino acid sequence set forth in SEQ NO:7; a light chain FR2 region; a CDR2 comprising the amino acid sequence set forth in SEQ ID NO:8; a light chain FR3 region; a CDR3 comprising the amino acid sequence set forth in SEQ ID NO:9; and a light chain FR4 region. In some of these embodiments, one or more of the FR regions is a humanized FR region. In some of these embodiments, each of the FR regions is a humanized FR region. The linker region can be from about 5 amino acids to about 50 amino acids in length, e.g., from about 5 aa to about 10 aa, from about 10 aa to about 15 aa, from about 15 aa to about 20 aa, from about 20 aa to about 25 aa, from about 25 aa to about 30 aa, from about 30 aa to about 35 aa, from about 35 aa to about 40 aa, from about 40 aa to about 45 aa, or from about 45 aa to about 50 aa in length.

Linkers suitable for use in an antibody include “flexible linkers”. If present, the linker molecules are generally of sufficient length to permit some flexible movement between linked regions. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to polypeptides may be used in light of this disclosure.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine polymers (including, for example, (GS)n, (GSGGS)n (SEQ ID NO: 49) and (GGGS)n (SEQ ID NO: 50), where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited GGSG (SEQ ID NO: 51), GGSGG (SEQ ID NO: 52), GSGSG (SEQ ID NO: 53), GSGGG (SEQ ID NO: 54), GGGSG (SEQ ID NO: 55), GSSSG (SEQ ID NO: 56), and the like. The ordinarily skilled artisan will recognize that design of a peptide conjugated to any elements described above can include linkers that are all or partially flexible, such that the linker can include a flexible linker as well as one or more portions that confer less flexible structure.

In some embodiments, an antibody suitable for use in a subject method is an antibody fragment, an Fv, scFv, Fab, F(ab′)2, or Fab′. Thus, the present disclosure provides an isolated antibody, wherein the antibody is a Fv, scFv, Fab, F(ab′)2, or Fab′, and wherein the antibody competes for binding to an epitope in an N-terminal region of a Tau polypeptide with an antibody that comprises: a) a light chain region comprising: i) a VL CDR1 comprising an amino acid sequence of SEQ ID NO:1 or SEQ ID NO:7; (ii) a VL CDR2 comprising an amino acid sequence of SEQ ID NO:2 or SEQ ID NO:8; and (iii) a VL CDR3 comprising an amino acid sequence of SEQ ID NO:3 or SEQ ID NO:9; and b) a heavy chain region comprising: (i) a VH CDR1 comprising an amino acid sequence of SEQ ID NO:4 or SEQ ID NO:10; (ii) a VH CDR2 comprising an amino acid sequence of SEQ ID NO:5 or SEQ ID NO:11; and (iii) a VH CDR3 comprising an amino acid sequence of SEQ ID NO:6 or SEQ ID NO:12. In some of these embodiments, the isolated antibody comprises one, two, three, or four humanized VL framework regions, as described above. In some of these embodiments, the isolated antibody comprises one, two, three, or four humanized VH framework regions, as described above.

In some embodiments, an antibody suitable for use in a subject method is a scFv antibody. In some embodiments, an anti-Tau antibody of the present disclosure comprises scFv multimers. For example, in some embodiments, a suitable antibody is an scFv dimer (e.g., comprises two tandem scFv (scFv2)), an scFv trimer (e.g., comprises three tandem scFv (scFv3)), an scFv tetramer (e.g., comprises four tandem scFv (scFv4)), or is a multimer of more than four scFv (e.g., in tandem). The scFv monomers can be linked in tandem via linkers of from about 2 amino acids to about 10 amino acids (aa) in length, e.g., 2 aa, 3 aa, 4 aa, 5 aa, 6 aa, 7 aa, 8 aa, 9 aa, or 10 aa in length. Suitable linkers include, e.g., (Gly)x, where x is an integer from 2 to 10. Other suitable linkers are those discussed above. In some embodiments, each of the scFv monomers in a scFV multimer is humanized, as described above.

In some embodiments, an antibody suitable for use in a subject method comprises a constant region of an immunoglobulin (e.g., an Fc region). The Fc region, if present, can be a human Fc region. If constant regions are present, the antibody can contain both light chain and heavy chain constant regions. Suitable heavy chain constant region include CH1, hinge, CH2, CH3, and CH4 regions. The antibodies described herein include antibodies having all types of constant regions, including IgM, IgG, IgD, IgA and IgE, and any isotype, including IgG1, IgG2, IgG3 and IgG4. An example of a suitable heavy chain Fc region is a human isotype IgG1 Fc. In some cases, the heavy chain region is of the isotype IgG4. In some of these embodiments, the hinge region comprises an S241P substitution. See, e.g., Angal et al. (1993) Mol. Immunol. 30:105. Light chain constant regions can be lambda or kappa. A suitable antibody (e.g., a humanized antibody) can comprise sequences from more than one class or isotype. Antibodies can be expressed as tetramers containing two light and two heavy chains, as separate heavy chains, light chains, as Fab, Fab′ F(ab′)2, and Fv, or as single chain antibodies in which heavy and light chain variable domains are linked through a spacer.

In some embodiments, an antibody suitable for use in a subject method comprises a human light chain constant region and a human heavy chain constant region, and wherein the isolated antibody competes for binding to an epitope in an N-terminal region of a Tau polypeptide with an antibody that comprises: a) a light chain region comprising: i) a VL CDR1 comprising an amino acid sequence of SEQ ID NO:1 or SEQ ID NO:7; (ii) a VL CDR2 comprising an amino acid sequence of SEQ ID NO:2 or SEQ ID NO:8; and (iii) a VL CDR3 comprising an amino acid sequence of SEQ ID NO:3 or SEQ ID NO:9; and b) a heavy chain region comprising: (i) a VH CDR1 comprising an amino acid sequence of SEQ NO:4 or SEQ ID NO:10; (ii) a VH CDR2 comprising an amino acid sequence of SEQ ID NO:5 or SEQ ID NO:11; and a VH CDR3 comprising an amino acid sequence of SEQ NO:6 or SEQ ID NO:12. In some of these embodiments, the isolated antibody comprises one, two, three, or four humanized VL framework regions, as described above. In some of these embodiments, the isolated antibody comprises one, two, three, or four humanized VH framework regions, as described above.

In some embodiments, an antibody suitable for use in a subject method can comprise a free thiol (—SH) group at the carboxyl terminus, where the free thiol group can be used to attach the antibody to a second polypeptide (e.g., another antibody, including a suitable antibody), a scaffold, a carrier, etc.

In some embodiments, an antibody suitable for use in a subject method comprises one or more non-naturally occurring amino acids. In some embodiments, the non-naturally encoded amino acid comprises a carbonyl group, an acetyl group, an aminooxy group, a hydrazine group, a hydrazide group, a semicarbazide group, an azide group, or an alkyne group. See, e.g., U.S. Pat. No. 7,632,924 for suitable non-naturally occurring amino acids. Inclusion of a non-naturally occurring amino acid can provide for linkage to a polymer, a second polypeptide, a scaffold, etc. For example, a suitable antibody linked to a water-soluble polymer can be made by reacting a water-soluble polymer (e.g., PEG) that comprises a carbonyl group to the antibody, where the antibody comprises a non-naturally encoded amino acid that comprises an aminooxy, hydrazine, hydrazide or semicarbazide group. As another example, a suitable antibody linked to a water-soluble polymer can be made by reacting a suitable antibody that comprises an alkyne-containing amino acid with a water-soluble polymer (e.g., PEG) that comprises an azide moiety; in some embodiments, the azide or alkyne group is linked to the PEG molecule through an amide linkage. A “non-naturally encoded amino acid” refers to an amino acid that is not one of the 20 common amino acids or pyrrolysine or selenocysteine. Other terms that may be used synonymously with the term “non-naturally encoded amino acid” are “non-natural amino acid,” “unnatural amino acid,” “non-naturally-occurring amino acid,” and variously hyphenated and non-hyphenated versions thereof. The term “non-naturally encoded amino acid” also includes, but is not limited to, amino acids that occur by modification (e.g. post-translational modifications) of a naturally encoded amino acid (including but not limited to, the 20 common amino acids or pyrrolysine and selenocysteine) but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine, and O-phosphotyrosine.

In some embodiments, an antibody suitable for use in a subject method is linked (e.g., covalently linked) to a polymer (e.g., a polymer other than a polypeptide). Suitable polymers include, e.g., biocompatible polymers, and water-soluble biocompatible polymers. Suitable polymers include synthetic polymers and naturally-occurring polymers. Suitable polymers include, e.g., substituted or unsubstituted straight or branched chain polyalkylene, polyalkenylene or polyoxyalkylene polymers or branched or unbranched polysaccharides, e.g. a homo- or hetero-polysaccharide. Suitable polymers include, e.g., ethylene vinyl alcohol copolymer (commonly known by the generic name EVOH or by the trade name EVAL); polybutylmethacrylate; poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g., poly(ethylene oxide)-poly(lactic acid) (PEO/PLA) co-polymers); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon; poly(ethylene glycol); and carboxymethyl cellulose.

Suitable synthetic polymers include unsubstituted and substituted straight or branched chain poly(ethyleneglycol), poly(propyleneglycol) poly(vinylalcohol), and derivatives thereof, e.g., substituted polyethyleneglycol) such as methoxypoly(ethyleneglycol), and derivatives thereof. Suitable naturally-occurring polymers include, e.g., albumin, amylose, dextran, glycogen, and derivatives thereof.

Suitable polymers can have an average molecular weight in a range of from 500 Da to 50000 Da, e.g., from 5000 Da to 40000 Da, or from 25000 to 40000 Da. For example, in some embodiments, where a suitable antibody comprises a poly(ethylene glycol) (PEG) or methoxypoly(ethyleneglycol) polymer, the PEG or methoxypoly(ethyleneglycol) polymer can have a molecular weight in a range of from about 0.5 kiloDaltons (kDa) to 1 kDa, from about 1 kDa to 5 kDa, from 5 kDa to 10 kDa, from 10 kDa to 25 kDa, from 25 kDa to 40 kDa, or from 40 kDa to 60 kDa.

As noted above, in some embodiments, a suitable antibody is covalently linked to a PEG polymer. In some embodiments, a scFv multimer is covalently linked to a PEG polymer. See, e.g., Albrecht et al. (2006) J. Immunol. Methods 310:100. Methods and reagents suitable for PEGylation of a protein are well known in the art and may be found in, e.g., U.S. Pat. No. 5,849,860, PEG suitable for conjugation to a protein is generally soluble in water at room temperature, and has the general formula R(O—CH2-CH2)nO—R, where R is hydrogen or a protective group such as an alkyl or an alkanol group, and where n is an integer from 1 to 1000. Where R is a protective group, it generally has from 1 to 8 carbons.

The PEG conjugated to the antibody can be linear. The PEG conjugated to the protein may also be branched. Branched PEG derivatives such as those described in U.S. Pat. No. 5,643,575, “star-PEG's” and multi-armed PEGs such as those described in Shearwater Polymers, Inc. catalog “Polyethylene Glycol Derivatives 1997-1998.” Star PEGs are described in the art including, e.g., in U.S. Pat. No. 6,046,305.

In some embodiments, an antibody suitable for use in a subject method can be glycosylated, e.g., a suitable antibody can comprise a covalently linked carbohydrate or polysaccharide moiety. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to an antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). Similarly, removal of glycosylation sites can be accomplished by amino acid alteration within the native glycosylation sites of an antibody.

A suitable antibody will in some embodiments comprise a “radiopaque” label, e.g. a label that can be easily visualized using for example x-rays. Radiopaque materials are well known to those of skill in the art. The most common radiopaque materials include iodide, bromide or barium salts. Other radiopaque materials are also known and include, but are not limited to organic bismuth derivatives (see, e.g., U.S. Pat. No. 5,939,045), radiopaque multiurethanes (see U.S. Pat. No. 5,346,981), organobismuth composites (see, e.g., U.S. Pat. No. 5,256,334), radiopaque barium multimer complexes (see, e.g., U.S. Pat. No. 4,866,132), and the like.

A suitable antibody can be covalently linked to a second moiety (e.g., a lipid, a polypeptide other than the antibody, a synthetic polymer, a carbohydrate, and the like) using for example, glutaraldehyde, a homobifunctional cross-linker, or a heterobifunctional cross-linker. Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one-step reaction procedure in which the cross-linker is added to a solution containing a mixture of the polypeptides to be linked. Homobifunctional NHS ester and imido esters cross-link amine containing polypeptides. In a mild alkaline pH, imido esters react only with primary amines to form imidoamides, and overall charge of the cross-linked polypeptides is not affected. Homobifunctional sulfhydryl reactive cross-linkers includes bismaleimidhexane (BMH), 1,5-difluoro-2,4-dinitrobenzene (DFDNB), and 1,4-di-(3′,2′-pyridyldithio) propinoamido butane (DPDPB).

Heterobifunctional cross-linkers have two or more different reactive moieties (e.g., amine reactive moiety and a sulfhydryl-reactive moiety) and are cross-linked with one of the polypeptides via the amine or sulfhydryl reactive moiety, then reacted with the other polypeptide via the non-reacted moiety. Multiple heterobifunctional haloacetyl cross-linkers are available, as are pyridyl disulfide cross-linkers. Carbodiimides are a classic example of heterobifunctional cross-linking reagents for coupling carboxyls to amines, which results in an amide bond.

A suitable antibody will in some embodiments comprise a detectable label, Suitably: detectable labels include any composition detectable by spectroscopic, photochemical, biochemical, immunochemical, electrical, optical or chemical means. Suitable include, but are not limited to, magnetic beads (e.g. Dynabeads™), fluorescent dyes (e.g., fluorescein isothiocyanate, texas red, rhodamine, a green fluorescent protein, a red fluorescent protein, a yellow fluorescent protein, and the like), radiolabels (e.g., ³H, ¹²⁵I, ³⁵S, ¹⁴C, or ³²P), enzymes (e.g., horse radish peroxidase, alkaline phosphatase, luciferase, and others commonly used in an enzyme-linked immunosorbent assay (ELISA)), and colorimetric labels such as colloidal gold or colored glass or plastic (e.g. polystyrene, polypropylene, latex, etc.) beads.

In some embodiments, a suitable antibody comprises a contrast agent or a radioisotope, where the contrast agent or radioisotope is one that is suitable for use in imaging, e.g., imaging procedures carried out on humans. Non-limiting examples of labels include radioisotope such as 1231I (iodine), 18F (fluorine), 99Tc (technetium). 111In (indium), and 67Ga (gallium), and contrast agent such as gadolinium (Gd), dysprosium, and iron. Radioactive Gd isotopes (153Gd) also are available and suitable for imaging procedures in non-human mammals. A suitable antibody can be labeled using standard techniques. For example, a suitable antibody can be iodinated using chloramine T or 1,3,4,6-tetrachloro-3α,6α-diphenylglycouril. For fluorination, fluorine is added to an anti-Tau antibody during the synthesis by a fluoride ion displacement reaction. See, Muller-Gartner, H., TIB Tech., 16:122-130 (1998) and Saji, H., Crit. Rev. Ther. Drug Carrier Syst., 16(2):209-244 (1999) for a review of synthesis of proteins with such radioisotopes. A suitable antibody can also be labeled with a contrast agent through standard techniques. For example, a suitable antibody can be labeled with Gd by conjugating low molecular Gd chelates such as Gd diethylene triamine pentaacetic acid (GdDTPA) or Gd tetraazacyclododecanetetraacetic (GdDOTA) to the antibody. See, Caravan et al., Chem. Rev, 99:2293-2352 (1999) and Lauffer et al., J. Magn. Reson. Imaging, 3:11-16 (1985). A suitable antibody can be labeled with Gd by, for example, conjugating polylysine-Gd chelates to the antibody. See, for example, Curtet et al., Invest. Radial., 33(10):752-761 (1998). Alternatively, a suitable antibody can be labeled with Gd by incubating paramagnetic polymerized liposomes that include Gd chelator lipid with avidin and biotinylated antibody. See, for example, Sipkins et al., Nature Med., 4:623-626 (1998).

Suitable fluorescent proteins that can be linked to a suitable antibody include, but are not limited to, a green fluorescent protein from Aequoria victoria or a mutant or derivative thereof e.g., as described in U.S. Pat. Nos. 6,066,476; 6,020,192; 5,985,577; 5,976,796; 5,968,750; 5,968,738; 5,958,713; 5,919,445; 5,874,304; e.g., Enhanced GFP, many such GFP which are available commercially, e.g., from Clontech, Inc.; a red fluorescent protein; a yellow fluorescent protein; any of a variety of fluorescent and colored proteins from Anthozoan species, as described in, e.g., Matz et al. (1999) Nature Biotechnol. 17:969-973; and the like.

An antibody will in some embodiments be linked to (e.g., covalently or non-covalently linked) a fusion partner, e.g., a ligand; an epitope tag; a peptide; a protein other than an antibody; and the like. Suitable fusion partners include peptides and polypeptides that confer enhanced stability in vivo (e.g., enhanced serum half-life); provide ease of purification, e.g., (His)n, e.g., 6His (SEQ ID NO: 57), and the like; provide for secretion of the fusion protein from a cell; provide an epitope tag, e.g., GST, hemagglutinin (HA; e.g., YPYDVPDYA; SEQ ID NO: 58), FLAG (e.g., DYKDDDDK; SEQ ID NO: 59), c-myc (e.g., EQKLISEEDL; SEQ ID NO: 60), and the like; provide a detectable signal, e.g., an enzyme that generates a detectable product (e.g., β-galactosidase, luciferase), or a protein that is itself detectable, e.g., a green fluorescent protein, a red fluorescent protein, a yellow fluorescent protein, etc.; provides for multimerization, e.g., a multimerization domain such as an Fc portion of an immunoglobulin; and the like.

The fusion may also include an affinity domain, including peptide sequences that can interact with a binding partner, e.g., such as one immobilized on a solid support, useful for identification or purification. Consecutive single amino acids, such as histidine, when fused to a protein, can be used for one-step purification of the fusion protein by high affinity binding to a resin column, such as nickel sepharose. Exemplary affinity domains include His5 (HHHHH) (SEQ ID NO: 61), HisX6 (HHHHHH) (SEQ ID NO: 57), C-myc (EQKLISEEDL) (SEQ ID NO: 60), Flag (DYKDDDDK) (SEQ ID NO: 59), StrepTag (WSHPQFEK) (SEQ ID NO: 62), hemagglutinin, e.g., HA Tag (YPYDVPDYA; SEQ ID NO: 58), glutathinone-S-transferase (GST), thioredoxin, cellulose binding domain, RYIRS (SEQ ID NO: 63), Phe-His-His-Thr (SEQ ID NO: 64), chitin binding domain, S-peptide, T7 peptide. SH2 domain, C-end RNA tag, WEAAAREACCRECCARA (SEQ ID NO: 65), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, leucine zipper sequences, and maltose binding protein.

A suitable antibody will in some embodiments be fused to a polypeptide that binds to an endogenous blood brain barrier (BBB) receptor. Linking a suitable antibody to a polypeptide that binds to an endogenous BBB receptor facilitates crossing the BBB, e.g., in a subject treatment method (see below) involving administration of a suitable antibody to an individual in need thereof. Suitable polypeptides that bind to an endogenous BBB receptor include antibodies, e.g., monoclonal antibodies, or antigen-binding fragments thereof, that specifically bind to an endogenous BBB receptor. Suitable endogenous BBB receptors include, but are not limited to, an insulin receptor, a transferrin receptor, a leptin receptor, a lipoprotein receptor, and an insulin-like growth factor receptor. See, e.g., U.S. Patent Publication No, 2009/0156498.

As an example, a suitable anti-Tau antibody can be a bi-specific antibody comprising a first antigen-binding portion that specifically binds an epitope in a Tau polypeptide; and a second antigen-binding portion that binds an endogenous BBB receptor. For example, in some instances, a suitable anti-Tau antibody is a hi-specific antibody comprising a first antigen-binding portion that specifically binds an epitope in a Tau polypeptide; and a second antigen-binding portion that binds a transferrin receptor.

For example, a suitable anti-Tau antibody can be fused to a peptide that facilitates crossing the BBB, the peptide having a length of from about 15 amino acids to about 25 amino acids, and comprising an amino acid sequence having at least about 85% amino acid sequence identity to one of the following peptides: Angiopep-1 (TFFYGGCRGKRNNFKTEEY; SEQ NO: 66); Angiopep-2 (TFFYGGSRGKRNNFKTEEY; SEQ ID NO: 67); cys-Angiopep-2 (CTFFYGGSRGKRNNFKTEEY; SEQ ID NO: 68); Angiopep-2-cys (TFFYGGSRGKRNNFKTEEYC; SEQ ID NO: 69); and an aprotinin fragment (TFVYGGCRAKRNNFKS; SEQ ID NO: 70). See, e.g., U.S. Patent Publication Nos. 2011/0288011; and 2009/0016959. A peptide that facilitates crossing the BBB can be fused to the N-terminus of an anti-Tau light chain region, to the C-terminus of an anti-Tau light chain region, to the N-terminus of an anti-Tau heavy chain region, to the C-terminus of an anti-Tau heavy chain region, to the N-terminus of an anti-Tau single-chain antibody, to the C-terminus of an anti-Tau single-chain antibody, etc.

In some embodiments, a suitable antibody comprises a polyamine modification. Polyamine modification of a suitable antibody enhances permeability of the modified antibody at the BBB. A suitable antibody can be modified with polyamines that are either naturally occurring or synthetic. See, for example, U.S. Pat. No. 5,670,477. Useful naturally occurring polyamines include putrescine, spermidine, spermine, 1,3-diaminopropane, norspermidine, syn-homospermidine, thermine, thermospermine, caldopentamine, homocaldopentamine, and canavalmine. Putrescine, spermidine and spermine are particularly useful. Synthetic polyamines are composed of the empirical formula CXHYNZ, can be cyclic or acyclic, branched or unbranched, hydrocarbon chains of 3-12 carbon atoms that further include 1-6 NR or N(R)2 moieties, wherein R is H, (C1-C4) alkyl, phenyl, or benzyl. Polyamines can be linked to an antibody using any standard crosslinking method.

In some embodiments, a suitable antibody is modified to include a carbohydrate moiety, where the carbohydrate moiety can be covalently linked to the antibody. In some embodiments, a suitable antibody is modified to include a lipid moiety, where the lipid moiety can be covalently linked to the antibody. Suitable lipid moieties include, e.g., an N-fatty acyl group such as N-lauroyl, N-oleoyl, etc.; a fatty amine such as dodecyl amine, oleoyl amine, etc.; a C3-C16 long-chain aliphatic lipid; and the like. See, e.g., U.S. Pat. No. 6,638,513). In some embodiments, a suitable antibody is incorporated into a liposome.

Combination Therapy

An anti-Tau antibody can be administered to an individual in need thereof alone (e.g., as monotherapy); or in combination therapy with one or more additional therapeutic agents. For example, an anti-Tau antibody can be administered in combination therapy with one or more additional therapeutic agents for the treatment of stroke, or for the treatment of TBI.

“In combination with” as used herein refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

Individuals to be Treated

Individuals suitable for treatment with an anti-Tau antibody include individuals who have been diagnosed as having a tauopathy (e.g., an acute tauopathy); individuals at greater risk than the general population for developing a tauopathy (e.g., individuals having a genetic predisposition to developing a tauopathy); military personnel; and the like. In some cases, individual is a human is from less than 10 years of age to 10 years of age; from 10 years of age to about 15 years of age; from about 15 years of age to about 20 years of age, or from about 20 years of age to about 30 years of age. In some cases, the individual is an adult human. In some cases, the adult human is from about 20 years of age to about 30 years of age; 30 years of age or older; 40 years of age or older, 50 years of age or older, 60 years of age or older, 70 years of age or older, or 80 years of age or older. For example, the adult human can be from 40 years old to 50 years old, from 50 years old to 60 years old, from 60 years old to 70 years old, or older than 70 years. In some cases, the individual is one who has TBI. In some cases, the individual is one who has had a stroke.

Formulations

In the subject methods, an anti-Tau antibody can be administered to the host using any convenient means capable of resulting in the desired therapeutic effect or diagnostic effect. Thus, the agent can be incorporated into a variety of formulations for therapeutic administration. More particularly, an anti-Tau antibody can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, an anti-Tau antibody can be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, an anti-Tau antibody can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An anti-Tau antibody can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Pharmaceutical compositions comprising an anti-Tau antibody are prepared by mixing the antibody having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, seine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration; see also Chen (1992) Drug Dev Ind Pharm 18, 1311-54.

Exemplary antibody concentrations in a pharmaceutical composition may range from about 1 mg/mL to about 200 mg/ml or from about 50 mg/mL to about 200 mg/mL, or from about 150 mg/mL to about 200 mg/mL.

An aqueous formulation of the antibody may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the antibody formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 nM.

A surfactant may also be added to the antibody formulation to reduce aggregation of the formulated antibody and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Plutonic® F68 or Poloxamer 188™. Examples of suitable Polyoxyethylene alkyl ethers are those sold under the trademark Brij™. Exemplary concentrations of surfactant may range from about 0.001% to about 1% w/v.

A lyoprotectant may also be added in order to protect the labile active ingredient (e.g. a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a suitable formulation includes an anti-Tau antibody, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a suitable formulation can be a liquid or lyophilized formulation suitable for parenteral administration, and can comprise: about 1 mg/mL to about 200 mg/mL of an anti-Tau antibody; about 0.001% to about 1% of at least one surfactant; about 1 mM to about 100 mM of a buffer; optionally about 10 mM to about 500 mM of a stabilizer; and about 5 mM to about 305 mM of a tonicity agent; and has a pH of about 4.0 to about 7.0.

As another example, a suitable parenteral formulation is a liquid or lyophilized formulation comprising: about 1 mg/mL to about 200 mg/mL of an anti-Tau antibody; 0.04% Tween 20 w/v; 20 mM L-histidine; and 250 mM Sucrose; and has a pH of 5.5.

As another example, a suitable parenteral formulation comprises a lyophilized formulation comprising: 1) 15 mg/mL of an anti-Tau antibody; 0.04% Tween 20 w/v; 20 mM L-histidine; and 250 mM sucrose; and has a pH of 5.5; or 2) 75 mg/mL of an anti-Tau antibody; 0.04% Tween 20 w/v; 20 mM L-histidine; and 250 mM sucrose; and has a pH of 5.5;or 3) 75 mg/mL of an anti-Tau antibody; 0.02% Tween 20 w/v; 20 mM L-histidine; and 250 mM Sucrose; and has a pH of 5.5; or 4) 75 mg/mL of an anti-Tau antibody; 0.04% Tween 20 w/v; 20 mM L-histidine; and 250 mM trehalose; and has a pH of 5.5; or 6) 75 mg/mL of an anti-Tau antibody; 0.02% Tween 20 w/v; 20 mM L-histidine; and 250 mM trehalose; and has a pH of 5.5.

As another example, a suitable parenteral formulation is a liquid formulation comprising:1) 7.5 mg/mL of an anti-Tau antibody; 0.022% Tween 20 w/v; 120 mM L-histidine; and 250 125 mM sucrose; and has a pH of 5.5; or 2) 37.5 mg/mL of an anti-Tau antibody; 0.02% Tween 20 w/v; 10 mM L-histidine; and 125 mM sucrose; and has a pH of 5.5; or 3) 37.5 mg/mL of an anti-Tau antibody; 0.01% Tween 20 w/v; 10 mM L-histidine; and 125 mM sucrose; and has a pH of 5.5; or 4) 37.5 mg/mL of an anti-Tau antibody; 0.02% Tween 20 w/v; 10 mM L-histidine; 12.5 mM trehalose; and has a pH of 5.5; or 5) 37.5 mg/mL of an anti-Tau antibody; 0.01% Tween 20 w/v; 10 mM L-histidine; and 125 mM trehalose; and has a pH of 5.5; or 6) 5 mg/mL of an anti-Tau antibody; 0.02% Tween 20 w/v; 20 mM L-histidine; and 250 mM trehalose; and has a pH of 5.5; or 7) 75 mg/mL of an anti-Tau antibody; 0.02% Tween 20 w/v; 20 mM L-histidine; and 250 mM mannitol; and has a pH of 5.5; or 8) 75 mg/mL of an anti-Tau antibody; 0.02% Tween 20 w/v; 20 mM L histidine; and 140 mM sodium chloride; and has a pH of 5.5; or 9) 150 mg/mL of an anti-Tau antibody; 0.02% Tween 20 w/v; 20 mM L-histidine; and 250 mM trehalose; and has a pH of 5.5; or 10) 150 mg/mL of an anti-Tau antibody; 0.02% Tween 20 w/v; 20 mM L-histidine; and 250 mM mannitol; and has a pH of 5.5; or 11) 150 mg/mL of an anti-Tau antibody; 0.02% Tween 20 w/v; 20 mM L-histidine; and 140 mM sodium chloride; and has a pH of 5.5; or 12) 10 mg/mL of an anti-Tau antibody; 0.01% Tween 20 w/v; 20 mM L-histidine; and 40 mM sodium chloride; and has a pH of 5.5.

An anti-Tau antibody can be utilized in aerosol formulation to be administered via inhalation. An anti-Tau antibody can be formulated into pressurized acceptable propellants such as dichlorodifluoromethane, propane, nitrogen and the like.

Furthermore, an anti-Tau antibody can be made into suppositories by mixing with a variety of bases such as emulsifying bases or water-soluble bases. An anti-Tau antibody can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise an anti-Tau antibody in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an anti-Tau antibody of the present disclosure, calculated in an amount sufficient to produce the desired effect in association with a pharmaceutically acceptable diluent, carrier or vehicle. The specifications for an anti-Tau antibody may depend on the particular antibody employed and the effect to be achieved, and the pharmacodynamics associated with each antibody in the host.

Other modes of administration will also find use with a method of the present disclosure. For instance, a suitable antibody can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), e.g., about 1% to about 2%.

Intranasal formulations will usually include vehicles that neither cause irritation to the nasal mucosa nor significantly disturb ciliary function. Diluents such as water, aqueous saline or other known substances can be employed. The nasal formulations may also contain preservatives such as, but not limited to, chlorobutanol and benzalkonium chloride. A surfactant may be present to enhance absorption of the antibody by the nasal mucosa.

An anti-Tau antibody can be administered as an injectable formulation. Typically, injectable compositions are prepared as liquid solutions or suspensions; solid forms suitable for solution in, or suspension in, liquid vehicles prior to injection may also be prepared. The preparation may also be emulsified or the antibody encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of an anti-Tau antibody adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an anti-Tau antibody is formulated in a controlled release formulation. Sustained-release preparations may be prepared using methods well known in the art. Suitable examples of sustained-release preparations include semipermeable matrices of solid hydrophobic polymers containing the antibody in which the matrices are in the form of shaped articles, e.g. films or microcapsules. Examples of sustained-release matrices include polyesters, copolymers of L-glutamic acid and ethyl-L-glutamate, non-degradable ethylene-vinyl acetate, hydrogels, polylactides, degradable lactic acid-glycolic acid copolymers and poly-D-(-)-3-hydroxybutyric acid. Possible loss of biological activity and possible changes in immunogenicity of antibodies comprised in sustained-release preparations may be prevented by using appropriate additives, by controlling moisture content and by developing specific polymer matrix compositions.

Controlled release within the scope of the present disclosure can be taken to mean any one of a number of extended release dosage forms. The following terms may be considered to be substantially equivalent to controlled release, for the purposes of the present disclosure: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.)

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

There are a number of controlled release drug formulations that are developed for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.).

Treatment Protocols

In one aspect, methods of treating a tauopathy (e.g., an acute tauopathy) in an individual are provided, the methods comprising administering to the individual an anti-Tau antibody.

Accordingly, in one embodiment, the dose of the anti-Tau antibody is calculated per mg/kg body weight. However, in another embodiment, the dose of the anti-Tau antibody is a flat-fixed dose that is fixed irrespective of the weight of the patient. In certain embodiments, dosage regimens are adjusted to provide the optimum desired response (e.g., an effective response).

In another embodiment, the dose of the anti-Tau antibody is varied over time. For example, the anti-Tau antibody may be initially administered at a high dose and may be lowered over time. In another embodiment, the anti-Tau antibody is initially administered at a low dose and increased over time.

In another embodiment, the amount of the anti-Tau antibody administered is constant for each dose. In another embodiment, the amount of antibody administered varies with each dose. For example, the maintenance (or follow-on) dose of the antibody can be higher or the same as the loading dose which is first administered. In another embodiment, the maintenance dose of the antibody can be lower or the same as the loading dose.

In one embodiment, the anti-Tau antibody is administered at dose of 2, 3, 4, 5, 6, 7, 8, 9, 10, 11, or 12 mg/kg. In one embodiment, the anti-Tau antibody is administered at dose of 10 mg/kg. In one embodiment, the anti-Tau antibody is administered at dose of 4 mg/kg. In another embodiment, the anti-Tau antibody is administered once. In another embodiment, more than one dose of the anti-Tau antibody are administered.

In other embodiments, the anti-Tau antibody is administered once per week, once every two or three weeks, once per month for as long as a clinical benefit is observed or, for example, until there is a complete response or unmanageable toxicity.

In another embodiment, the anti-Tau antibody is administered as a first line of treatment (e.g., the initial or first treatment). In another embodiment, the anti-Tau antibody is administered as a second line of treatment (e.g., after the initial or first treatment, including after relapse and/or where the first treatment has failed).

The following examples are merely illustrative and should not be construed as limiting the scope of this disclosure in any way as many variations and equivalents will become apparent to those skilled in the art upon reading the present disclosure.

The contents of all references, Genbank entries, patents and published patent applications cited throughout this application are expressly incorporated herein by reference.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Effect of IPN002 on Tau Levels and on Aβ Levels

Male cynomolgus monkeys (Macaca fascicularis) were given a single slow bolus injection of IPN002 at a dose level of 20 mg/kg and plasma and cerebrospinal fluid (CSF) samples collected at various time-points following injection. All samples (CSF and plasma) were measured for the presence of IPN002 using a specific Tau capture ELISA assay. This assay is only able to detect IPN002 that is not bound to Tau. In addition, Tau and Aβ levels were measured in CSF using commercially available ELISA assays. The capture antibody used in the Tau assay (Invitrogen) competes with IPN002 and therefore the assay only reports the level of free Tau (i.e. only Tau that is not bound to IPN002).

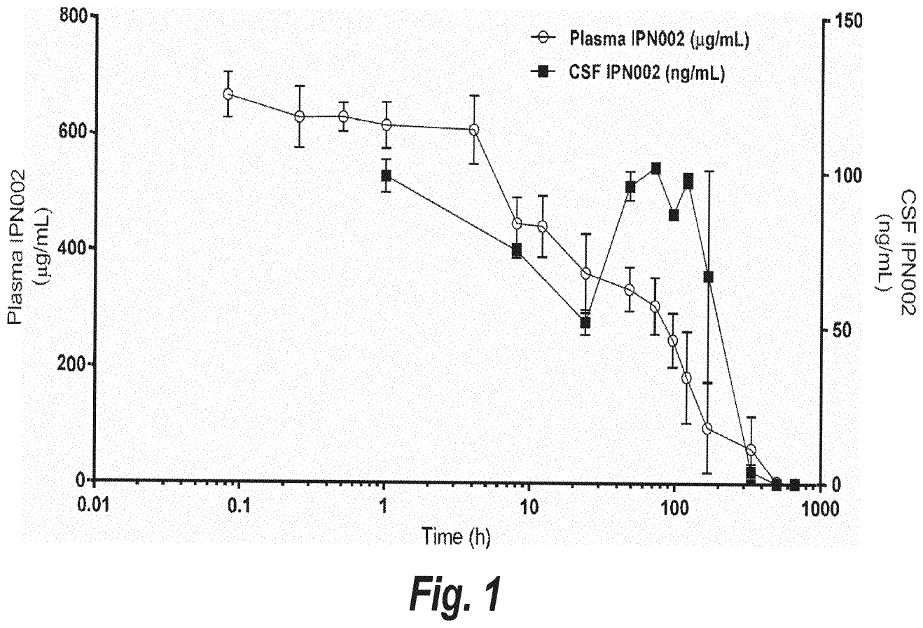

As shown in FIG. 1, the maximum concentration of IPN002 in plasma was achieved shortly after injection (approximately 666 μg/mL at 5 minutes post injection) and remained relatively constant for 8 hours after which the antibody was cleared from plasma with the expected kinetics. Surprisingly, IPN002 was detected in CSF at the earliest time-point examined (1 hour, see FIG. 1) but at much lower levels than observed in plasma. IPN002 levels in CSF tracked with plasma levels for the first 24 hours post injection but then remained relatively constant for 168 hours.

FIG. 1. Measurement of IPN002 in the CSF and plasma of Cynomolgus monkeys after a single injection of IPN002 at a dose level of 20 mg/kg. IPN002 was measured using a specific ELISA assay. Values represent the average of all samples collected at specific time-points (mean±standard deviation).

Consistent with the observation that IPN002 was rapidly detectable in CSF, Tau levels were also significantly decreased within 1 hour of IPN002 injection (FIG. 2). Indeed, no free Tau was detectable in CSF 8 hours following injection and this effect persisted for 168 hours, consistent with the pharmacokinetics of IPN002 in the CSF.

FIG. 2. Measurement of IPN002 and Tau in the CSF of Cynomolgus monkeys after a single injection of IPN002 at a dose level of 20 mg/kg. IPN002 was measured using a specific ELISA assay. Values represent the average of all samples collected at specific time-points (mean±standard deviation). Tau protein was measured using a commercially available ELISA assay (Invitrogen) and values represent the average of all samples collected at specific time-points (mean±standard error of the mean). Note that the CSF samples collected 7 days prior to IPN002 injection (Day −7) are plotted on the graph for reference.

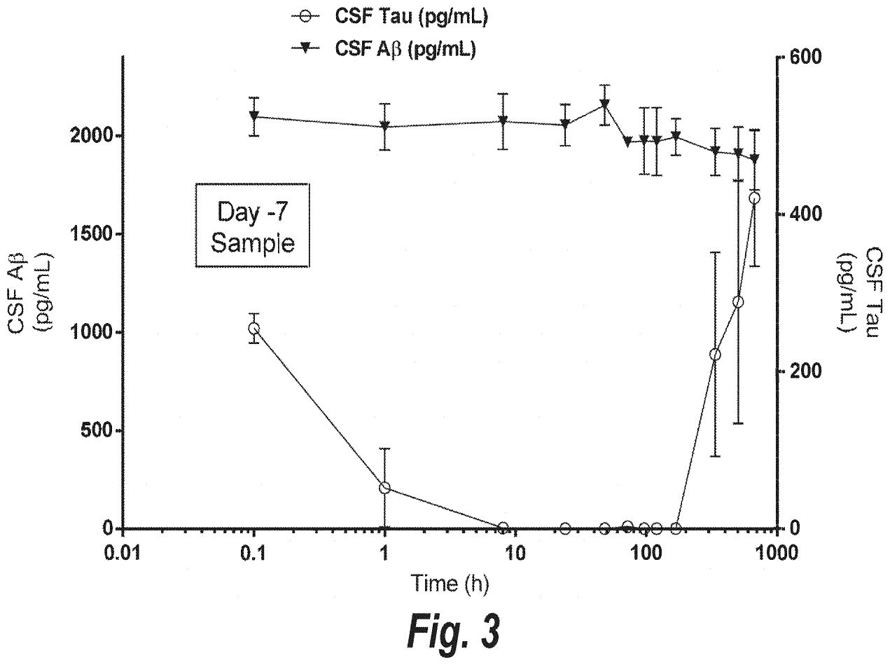

In contrast, levels of Aβ protein in the CSF were not significantly changed under the conditions tested (FIG. 3).

FIG. 3. Measurement of Aβ and Tau in the CSF of Cynomolgus monkeys after a single injection of IPN002 at a dose level of 20 mg/kg. Tau and Aβ protein were measured using commercially available ELISA assays and values represent the average of all samples collected at specific time-points (mean±standard error of the mean). Note that the CSF samples collected 7 days prior to IPN002 injection (Day −7) are plotted on the graph for reference.

### Example 2: Effect of hu-IPN002 on Tan Levels and on Aβ Levels

Male cynomolgus monkeys (Macaca fascicularis) were given a humanized variant of IPN002 (“hu-IPN002”) in a single slow bolus injection at a dose level of 5 mg/kg or 20 mg/kg.

Analysis of Serum and CSF hu-IPN002 Concentrations

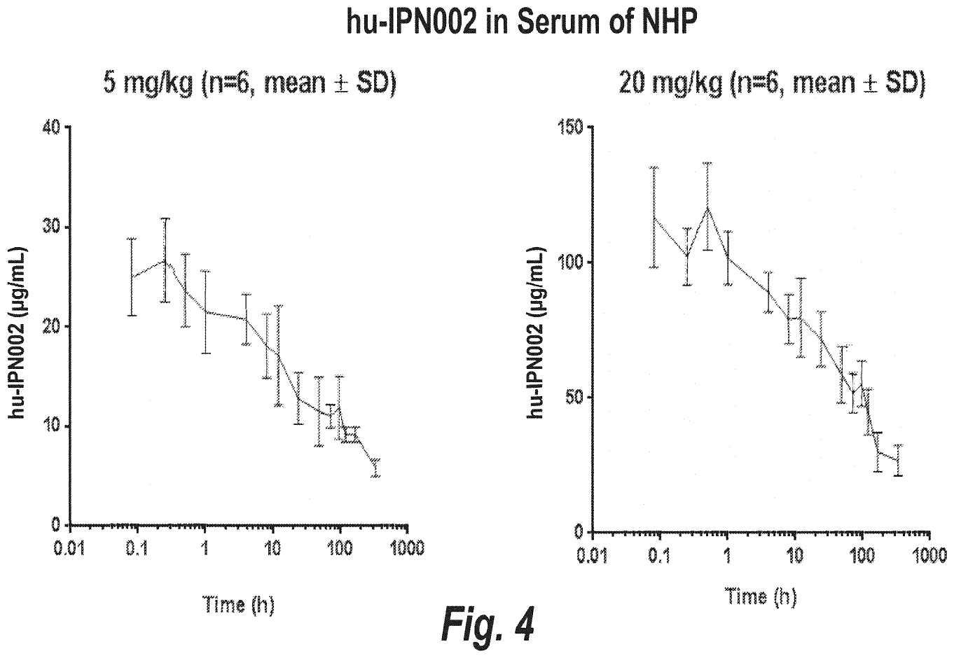

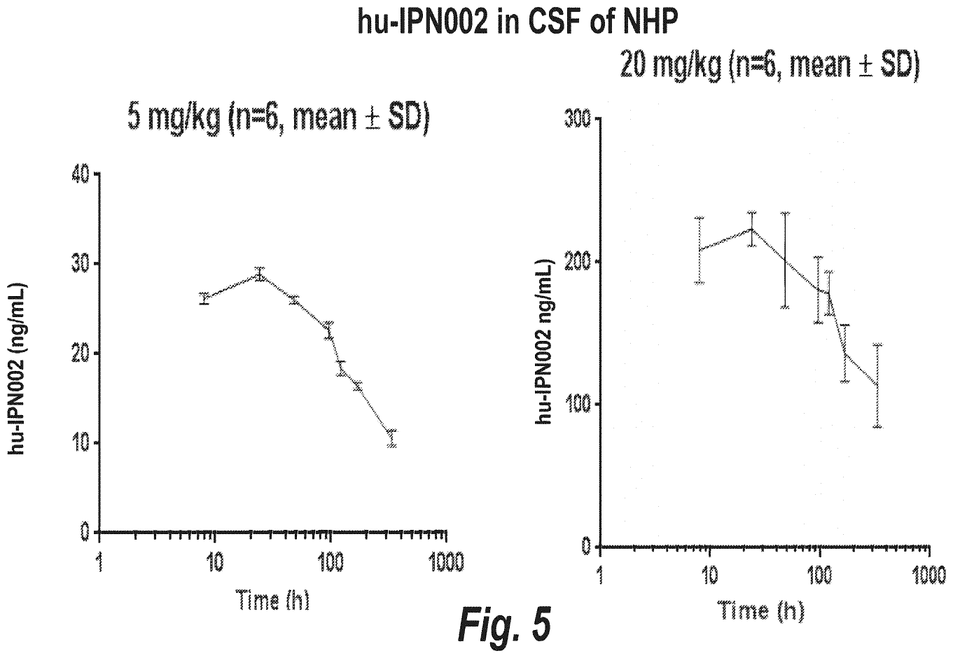

The level of hu-IPN002 in serum and in CSF was assayed. The results are shown in FIGS. 4A and 4B, and in FIGS. 5A and 5B.

As shown in FIG. 4A, administration of 5 mg/kg hu-IPN002 resulted in levels of hu-IPN002 in the serum of about 25 μg/ml within about 0.1 hour. As shown in FIG. 4B, administration of 20 mg/kg hu-IPN002 resulted in levels of hu-IPN002 in the serum of about 120 μg/ml within about 0.1 hour.

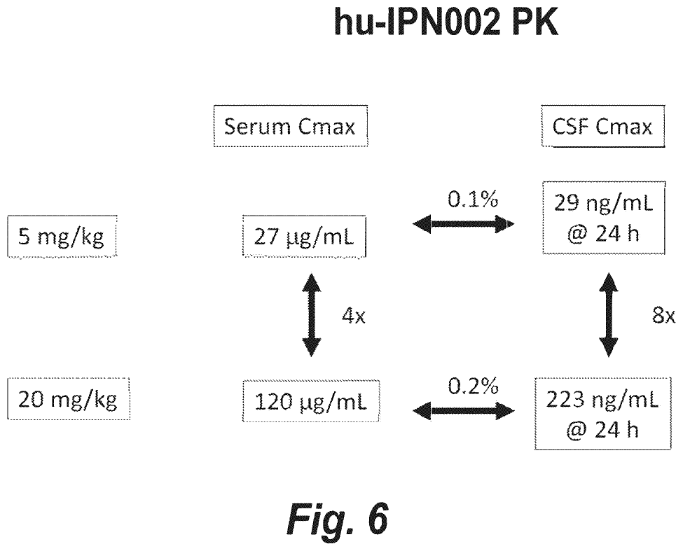

As shown in FIG. 5A, administration of 5 mg/kg hu-IPN002 resulted in levels of hu-IPN002 in the CSF of about 25 ng/ml at the 10-hour time point. As shown in FIG. 5B, administration of 20 mg/kg hu-IPN002 resulted in levels of hu-IPN002 in the CSF of about 200 ng/ml at the 10-hour time point. The pharmacokinetic data are summarized in FIG. 6.

Analysis of Free Tan Levels in CSF

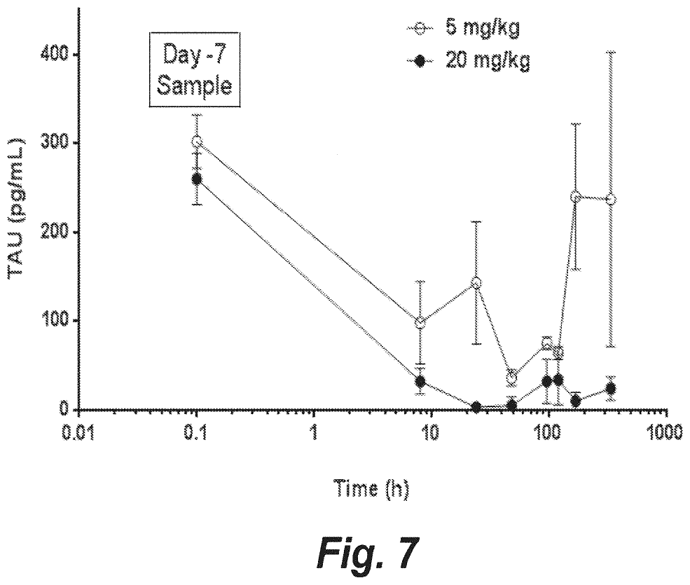

The effect of hu-IPN002 on free Tau levels in the CSF was tested. Male cynomolgus monkeys were treated as described above, and the level of free Tau levels in CST was measured. The results are shown in FIG. 7. As shown in FIG. 7, a single injection of 5 mg/kg or 20 mg/kg hu-IPN002 reduced free Tau levels in the CSF. Tau levels remained low for over 160 hours following administration of the hu-IPN002 antibody.

Analysis of Aβ Levels in CSF

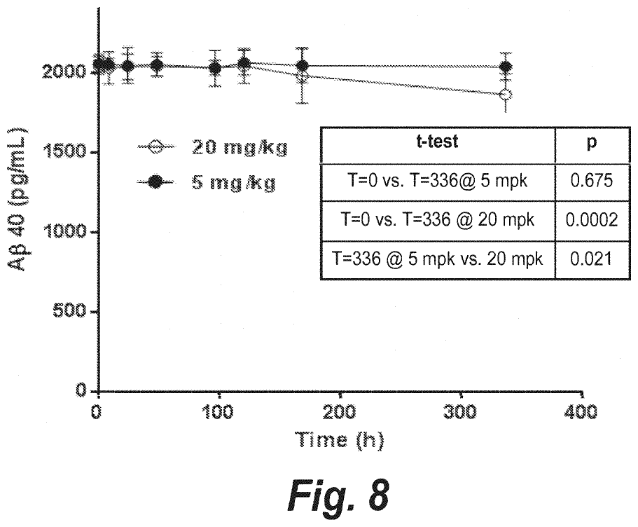

The effect of hu-IPN002 on Aβ levels in CSF of non-human primates was assessed. Male cynomolgus monkeys (Macaca fascicularis) were given a single slow bolus injection of hu-IPN002 at a dose level of 5 mg/kg or 20 mg/kg. Cerebrospinal fluid (CSF) samples were collected at various time-points following injection. CSF samples were measured for the presence of Aβ40 using a commercially available ELISA assay. The results are shown in FIG. 8. Values represent the average of all samples collected at specific time-points (mean±standard error of the mean).

As shown in FIG. 8, a single injection of 20 mg/kg hu-IPN002 reduced the level of Aβ40 in CSF after about 150 hours. The level of Aβ40 in CSF continued to drop up to about 350 hours.

### Example 3: Tau Fragments are Present in CSF Obtained from Individuals with Likely Chronic Traumatic Encephalopathy (CTE)

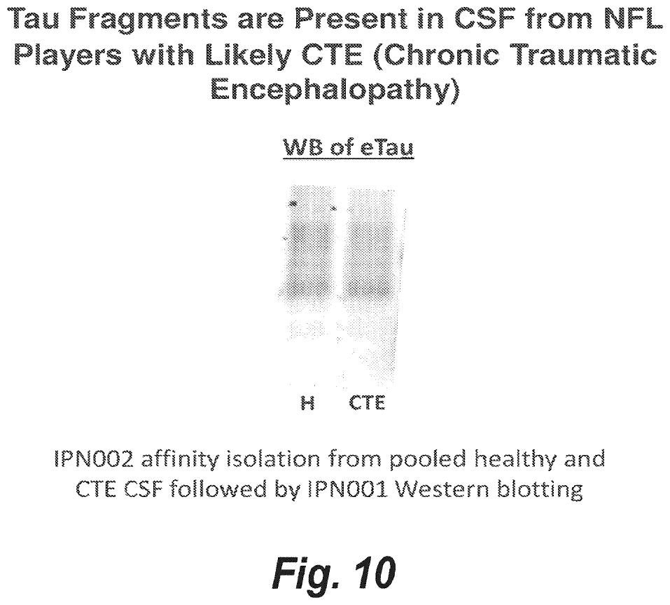

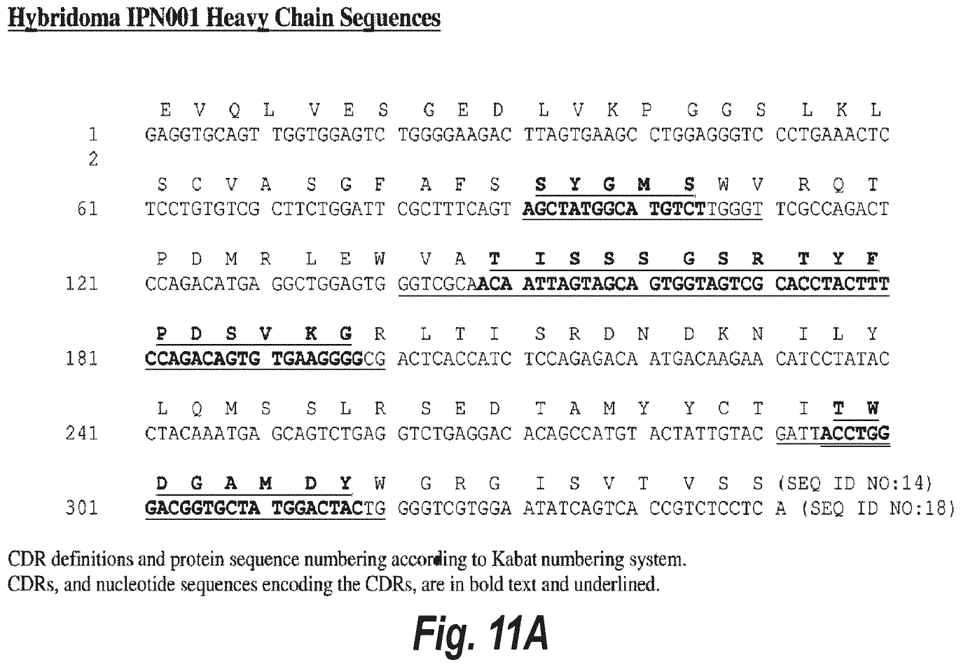

CSF samples were obtained from former National Football League linemen, who exhibited behavioral/cognitive deficits, and who were considered likely to have CTE. The CSF samples were assayed for the presence of eTau fragments. eTau fragments were affinity isolated from pooled CSF from healthy individuals and individuals with likely CTE. The isolated eTau fragments were separated using polyacrylamide gel electrophoresis; and the separated fragments were transferred to a membrane. The membrane was probed with IPN001. The results, presented in FIG. 10, show that Tau fragments are present in CSF obtained from individuals with likely CTE.

### Example 4: Effect of hu-IPN002 on Tan Levels and on Aβ Levels (Extended Single Intravenous Dose Study—5 mg/kg or 20 mg/kg)

Male cynomolgus monkeys were given hu-IPN002 in a single slow bolus injection at a dose level of 5 mg/kg or 20 mg/kg. Blood was obtained from all animals at predose, and at 0.083, 0.25, 0.5, 1, 4, 8, 12, 24, 48, 72, 96, 120, 168, 312 (Day 14), 480 (Day 21), 648 (Day 28), 816 (Day 35), 984 (Day 42), 1152 (Day 49), and 1320 (Day 56) hours following a single dose on Day 1 for analysis of serum hu-IPN002. CSF was obtained from all animals at predose and from animal cohorts at 8, 24, 48, 96, 120, and 168, 312 (Day 14), 480 (Day 21), 648 (Day 28), 816 (Day 35), 984 (Day 42), 1152 (Day 49), and 1320 (Day 56) hours for analysis of CSF hu-IPN002. The level of hu-IPN002 in serum and in CSF was assayed using enzyme linked immunosorbent assays (ELISA).

Analysis of Serum and CSF hu-IPN002 Concentrations

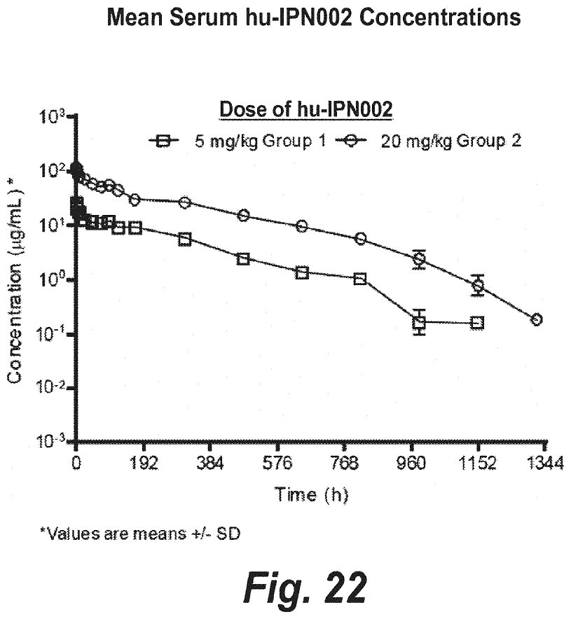

The pharmacokinetic summary for serum hu-IPN002 is shown in Table 4 below and the serum hu-IPN002 concentration versus time profile is shown in FIG. 22.

TABLE 4
Mean Serum hu-IPN002 Pharmacokinetic Parameters

hu-IPN002 (mg/kg)

| | |
|---|---|
| 5 | 20 |

| | |
|---|---|
| Parameter | Males |

| | | |
|---|---|---|
| AUC (0-T): μg · h/mL | 4,340^{a} | 21,000^{b} |
| AUC (INF): μg · h/mL | 4,410 | 21,100 |
| Cmax: μg/mL | 27.7 | 130 |
| Tmax: h | 2.2 | 0.36 |
| CLT: ml/h/kg | 1.15 | 0.964 |
| T-HALF: h | 170 | 150 |
| Vss: L/kg | 0.293 | 0.271 |

For T-HALF, value is harmonic mean
^{a}The mean AUC (0-T) value was calculated by averaging AUC (0-816 h), AUC (0-984 h), and AUC (0-1152 h) values.
^{b}The mean AUC (0-T) value was calculated by averaging AUC (0-984 h), AUC (0-1152 h), and AUC (0-1320 h) values.

After a single intravenous dose, the mean hu-IPN002 systemic exposures (AUC[0-T] and AUC[INF]) increased approximately dose proportionally between 5 and 20 mg/kg. Mean CL values were 1.15 and 0.964 mL/h/kg and mean Vss values were 0.293 and 0.271 L/kg for 5 and 20 mg/kg doses, respectively. The mean T-HALF values were 170 and 150 hours for 5 and 20 mg/kg, respectively.

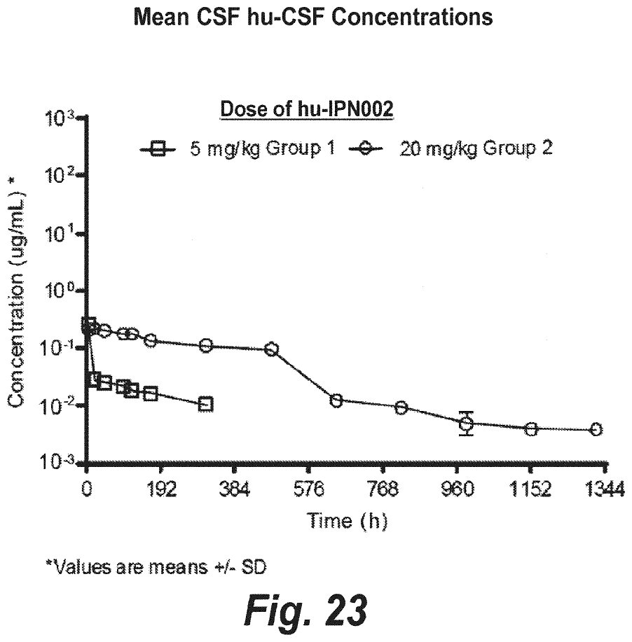

The pharmacokinetic summary for CSF hu-IPN002 is shown in Table 5 below and the CSF hu-IPN002 concentration versus time profile is showed in FIG. 23.

TABLE 5
Mean CSF hu-IPN002 Pharmacokinetic Parameters

hu-IPN002 (mg/kg)

| | |
|---|---|
| 5 | 20 |

| | |
|---|---|
| Parameter | Males |

| | | |
|---|---|---|
| AUC (0-T): μg · h/mL | 5.55^{a} | 79.8^{b} |
| AUC (INF): μg · h/mL | N/A | 80.8 |
| Cmax: μg/mL | 0.0277 | 0.217 |
| Tmax: h | 23 | 23 |
| T-HALF: h | 210 | 190 |
| CSF/Serum AUC (0-T) Ratio | 0.0013 | 0.0038 |
| CSF/Serum AUC (INF) Ratio | N/A | 0.0039 |

For T-HALF, value is harmonic mean
N/A = Not applicable due to insufficient data
^{a}The mean systemic exposure was averaged from individual AUC (0-312 h).
^{b}The mean systemic exposure was averaged from individual AUC (0-1320 h).

After a single intravenous dose, hu-IPN002 was detected in monkey CSF at the earliest time point (8 hours post dose), and the mean maximum CSF hu-IPN002 concentrations were achieved at 23 hours post dose. The CSF T-HALF values were similar (1.2 to 1.3×) to those values in serum. The mean CSF hu-IPN002 exposures (AUC[0-T]) increased greater than dose proportionally between 5 and 20 mg/kg. The mean CSF/Serum AUC (0-T) ratios were 0.0013 and 0.0038 for 5 and 20 mg/kg, respectively. While the CSF AUC (INF) value was not reportable for 5 mg/kg due to insufficient data, the CSF AUC (INF) value of 20 mg/kg was 0.0039× the corresponding serum AUC (INF) value.

Analysis of Free Tau Levels in CSF

The effect of hu-IPN002 on free Tau levels in the CSF was also tested. Male cynomolgus monkeys were treated as described above, and the level of free Tau levels in CSF was measured using a commercial ELISA kit. The results are shown in FIG. 11, which depicts the CSF free eTau levels (percentage of baseline) versus time profile.

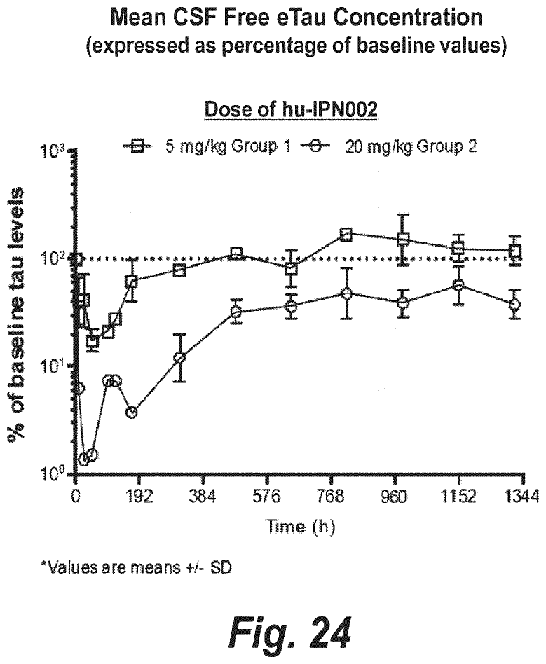

As shown in FIG. 24, after a single intravenous dose of hu-IPN002, CSF free eTau levels were reduced in a dose dependent manner at the earliest time point (8 hours post dose), with maximal reductions of 83 and 99% at 5 and 20 mg/kg, respectively. At the 5 mg/kg dose, maximal target engagement (minimal free eTau) was reached between 48 and 96 hours, with a free eTau level of 17.3-21% of baseline. Free eTau levels returned to baseline at approximately 480 hours (Day21) post single intravenous dose. In contrast, eTau levels at 20 mg/kg remained lower than baseline throughout the 8-week post-dose period. At the 20 mg/kg dose, maximal target engagement was observed between 8 and 168 hours, with a free eTau level of 1.35-7.44% of baseline. While free eTau levels remained reduced relative to baseline throughout the study period of 1320 hours, concentrations were increasing towards baseline at the later time points.

Analysis if Aβ Levels in CSF

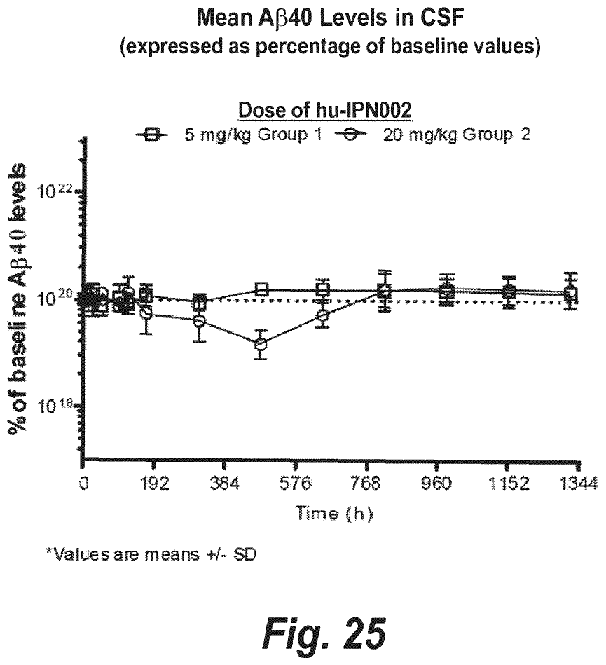

The effect of hu-IPN002 on Aβ levels in CSF of male cynomolgus monkeys (Macaca fascicularis) was also assessed. Male cynomolgus monkeys were treated as described above and CSF samples were collected at various time-points following injection, CSF samples were measured for the presence of Aβ40 using a commercially available ELISA assay. The results are shown in FIG. 25, which depicts the CSF Aβ40 levels (percentage of baseline) versus time profile. No changes were observed in CSF Aβ40 levels in the 5 mg/kg dose group. In contrast, in the 20 mg/kg group, CSF Aβ40 levels were reduced to an average of 82% of baseline at 480 hours. By 816 hours and for the remainder of the study period, the CSF Aβ40 levels returned to baseline. CSF Aβ40 levels were significantly reduced by 17% versus baseline in the 20 mg/kg group at 3 weeks post dose, but returned to baseline at 648 hours.

### Example 5 Effect of hu-IPN002 on Tau Levels and on Aβ Levels (Single Intravenous Dose Study—0.5 mg/kg, 2.0 mg/kg, 5.0 mg/kg, or 20 mg/kg)

A single dose, multiple dose level, intravenous (IV) bolus infusion study was performed to evaluate the serum and CSF pharmacokinetic and pharmacodynamic profile of hu-IPN002 over a 57 day time period. The dose levels employed were 0.5, 2, 5 and 20mg/kg. The pharmacodynamic endpoints included free CSF eTau and Abeta 42.

Eleven male cynomolgus monkeys had been previously implanted with vascular access ports (femoral vein and femoral artery) and cerebrospinal fluid (CSF) lumbar access ports (catheters ending at L1). Each had been used previously in small molecule pharmacological studies, although there was a drug-free period of at least one month prior to these studies. The monkeys were approximately 5-9 years of age and weighed 4.6-8.7 kg at the start of the study. Subjects were typically pair-housed and fed standard monkey chow (Harlan Teklad. Global 20% protein Primate Diet 2050) except for the morning before an infusion. Water was continuously available and fresh fruit was provided twice weekly. Toys and foraging devices were routinely provided and television programs were available in the colony rooms, Laboratory animal care was according to U.S. Public Health Service Policy on the Humane Care and Use of Laboratory. Animals, and Guide for the Care and use of Laboratory Animals, (2011).

Baseline measures of each analyte were determined from multiple CSF samples prior to the beginning of the study. The study began with a vehicle administration to each animal. Vehicle was administered as a single slow bolus volume of 6 mL/kg over 20 min through the venous access port. The vehicle was 0.02% Tween-80 in pH 5.8 PBS consisting of 10 mM phosphate and 140 mM NaCl. Blood and CSF were sampled for at least two weeks following vehicle and prior to the administration of hu-IPN002 on the following schedule: Serum sampling time points were: pre-dose, 0.5, 1, 2, 4, 8, 24, 48, 72, 168, 336 hr post-infusion through the arterial access port (times are relative to the end of the infusion). CSF sampling time points were: 2, 4, 7, 8, 24, 25, 48, 49, 72, 73, 168, 169, 336 and 337 hr post infusion. Treatment groups were assigned as shown in Table 6. hu-IPN002 was administered as a single slow bolus dose of 0.5, 2.0, 5.0 or 20.0 mg/kg in a dose volume of 6 mL/kg over 20 min through the venous access port. Serum sampling time points were: pre-dose, 0.5, 1, 2, 4, 8, 24, 48, 72, 168, 336, 504, 672, 840, 1008, 1176, 1344, and 1512 hr post-dose through the arterial access port (times are relative to the end of the infusion). CSF sampling time points were: 2, 4, 7, 8, 24, 25, 48, 49, 72, 73, 168, 169, 336, 337, 504, 505, 672, 673, 840, 841, 1008, 1009, 1176, 1177, 1344, 1345, 1512 and 1513 hr. CSF samples from 8, 25, 49, 73 169, 337, 505, 673, 841, 1009, 1177, 1345 and 1513 hr were used for interim analyses, other samples were analyzed in a single batch at the end of the study.

TABLE 6
Study Design; Single-Dose IPN001 in Cannulated Cynomolgus Monkeys

| | | | | Dose | |
|---|---|---|---|---|---|
| | Test | Dose Level | | Volume | |
| Group | Article | (mg/kg) | Route | (mL/kg) | # Monkeys |
| 1 | vehicle | 0 | IV | 6 | 1 |
| 2 | hu-IPN002 | 0.5 | IV | 6 | 3 |
| 3 | hu-IPN002 | 2.0 | IV | 6 | 2 |
| 4 | hu-IPN002 | 5.0 | IV | 6 | 3 |
| 5 | hu-IPN002 | 20.0 | IV | 6 | 2 |

Analysis of Serum and CSF hu-IPN002 Concentrations

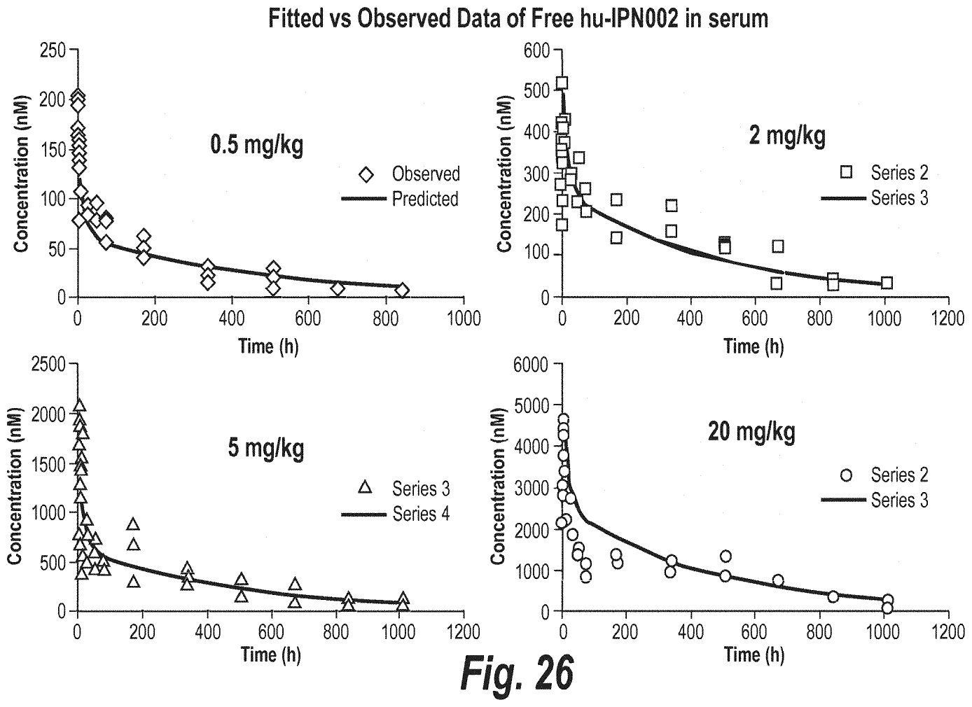

hu-IPN002 levels were measured in both serum and CSF samples using a specific ELISA. FIG. 26 shows the fitted vs observed data of hu-IPN002 in serum and FIG. 27 shows the fitted vs observed data of hu-IPN002 in CSF.

As shown in FIG. 26, AUC[INF] of hu-IPN002 increased in a dose-proportional fashion from 0.5 mg/kg to 20 mg/kg (4131, 20192, 47087 and 145300 μg·h/mL at 0.5, 2, 5 and 20 mg/kg, respectively). The mean serum half-life [T-HALF] values ranged from 218 to 276 h. The mean serum clearance [CL] was calculated to be 0.12 mL/h/kg. The Vss values ranged from 0.037 to 0.059 L/kg.

As shown in FIG. 27, hu-IPN002 concentrations in the CSF also increased in a dose-proportional fashion, where the AUC[0-T] was 0.1% of the corresponding serum AUC[0-T] of hu-IPN002, except at the 0.5 mg/kg dose in which CSF exposure appeared to be lower than predicted by serum concentrations (AUC[0-T] in CSF was determined to be 0.05% of the serum AUC[0-T]).

Analysis of Free Tan Levels in CSF

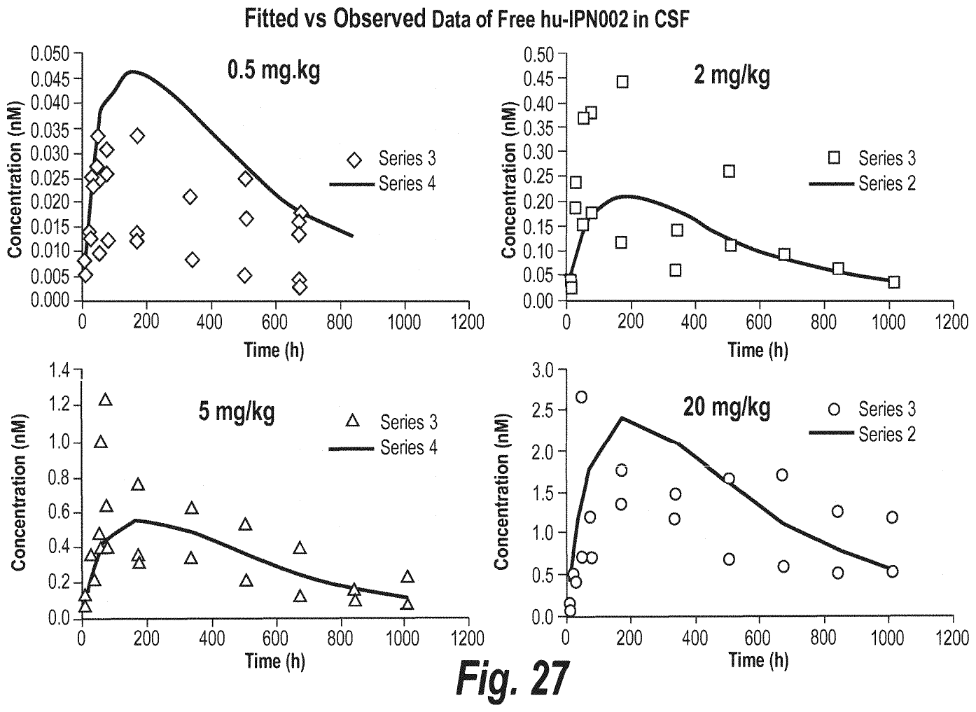

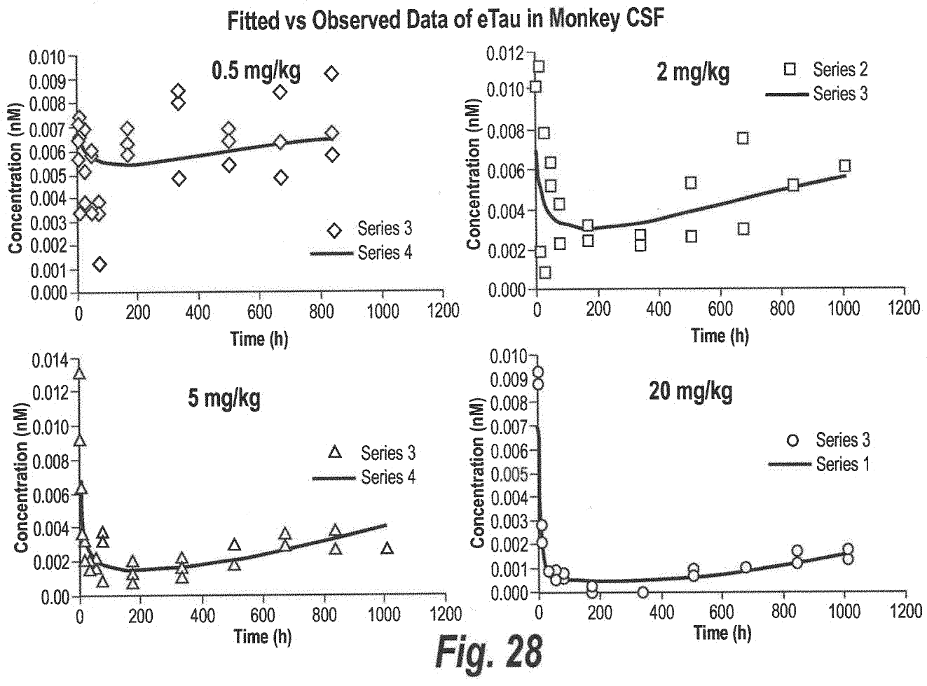

The effect of hu-IPN002 on free eTau levels in CSF was measured using an ELISA. FIG. 28 shows the fitted vs observed data of hu-IPN002 eTau in CSF. The Kdeg for degradation of eTau was estimated to be 0.11 h−1 The Kd was estimated to be 0.16 nmol/L.

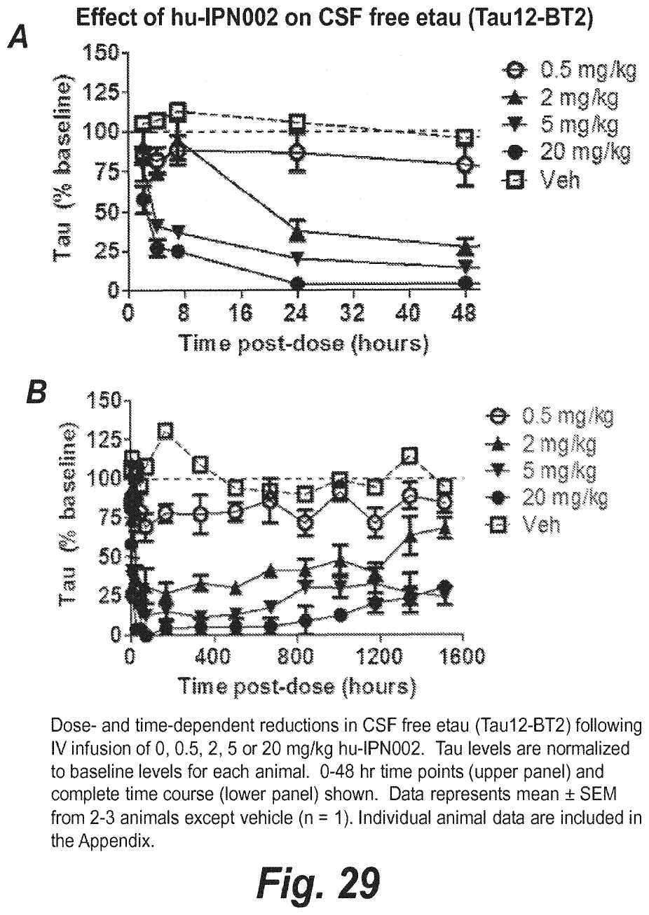

As shown in FIGS. 29A-29B and Table 6 below, hu-IPN002 induced dose- and time-dependent reductions in free eTau.

TABLE 6
Effect of hu-IPN002 on CSF free eTau (Tau12-BT2

| Time | | 0.5 | | | | | | | | | 20 | | |
|---|---|---|---|---|---|---|---|---|---|---|---|---|---|
| post- | | mpk | | | 2 mpk | | | 5 mpk | | | mpk | | |
| dose | Veh | (n = 3) | | | (n = 2) | | | (n = 2) | | | (n = 2) | | |
| (hr) | (n = 1) | Mean | SD | CV | Mean | SD | CV | Mean | SD | CV | Mean | SD^{a} | CV |
| 2.0 | 105.2 | 85.3 | 9.7 | 11 | 91.1 | 14.8 | 16 | 79.2 | 13.6 | 17 | 58.0 | 12.3 | 21 |
| 4.0 | 106.9 | 81.9 | 13.9 | 17 | 74.6 | 4.6 | 6 | 41.1 | 1.8 | 4 | 27.0 | 7.5 | 28 |
| 7.0 | 112.8 | 88.3 | 16.1 | 18 | 94.5 | 17.2 | 18 | 36.9 | 1.8 | 5 | 24.9 | 5.9 | 24 |
| 24.0 | 105.7 | 86.7 | 20.3 | 23 | 37.9 | 8.9 | 24 | 20.2 | 4.2 | 21 | 8.3 | NA | |
| 48.0 | 95.3 | 78.8 | 22.1 | 28 | 27.4 | 7.2 | 26 | 14.1 | 1.8 | 13 | 8.7 | NA | |
| 72.0 | 107.8 | 69.3 | 15.3 | 22 | 31.5 | 15.6 | 50 | 12.8 | 1.4 | 11 | <LLQ | NA | |
| 168 | 130.2 | 77.9 | 9.6 | 12 | 26.7 | 9.5 | 36 | 15.6 | 7.3 | 47 | 9.3 | NA | |
| 336 | 109.1 | 77.2 | 21.4 | 28 | 33.4 | 6.5 | 20 | 11.5 | 0.6 | 5 | 10.7 | NA | |
| 504 | 94.1 | 78.9 | 10.1 | 13 | 30.6 | 4.4 | 14 | 13.4 | 3.8 | 28 | 10.8 | NA | |
| 672 | 91.6 | 85.8 | 24.6 | 29 | 41.3 | 2.0 | 5 | 17.6 | 2.5 | 14 | 10.8 | NA | |
| 840 | 89.9 | 71.5 | 13.9 | 19 | 41.6 | 9.9 | 24 | 30.7 | 5.7 | 19 | 18.3 | NA | |
| 1008 | 98.8 | 91.2 | 9.8 | 11 | 47.5 | 13.4 | 28 | 30.2 | 9.2 | 30 | 12.7 | 1.8 | 14 |
| 1176 | 94.4 | 71.4 | 16.4 | 23 | 39.9 | 7.9 | 20 | 32.7 | 13.9 | 42 | 20.4 | 8.9 | 43 |
| 1344 | 114.2 | 88.7 | 14.7 | 17 | 63.3 | 17.4 | 27 | 26.9 | 18.1 | 67 | 24.0 | 7.1 | 30 |
| 1512 | 94.0 | 84.2 | 11.0 | 13 | 68.0 | 9.6 | 14 | 24.7 | 8.1 | 33 | 30.3 | 0.8 | 3 |

“LLQ” refers to the lower limit of quantitation of the ELISA.

As shown in FIGS. 29A-29B, hu-IPN002 reduced CSF free eTau in a dose- and time-dependent fashion. For example, at 24 hours post-dose, free eTau levels were reduced to 8.67% baseline, 37.9% baseline, 20.2% baseline and 8.3% baseline following 0.5, 2, 5 and 20 mg/kg IV doses, respectively. At 48 hours post-dose, free eTau levels were reduced to 78.8% baseline, 27.4% baseline, 14.1% baseline and 8.7% baseline following 0.5, 2, 5 and 20 mg/kg IV doses, respectively. At 72 hours post-dose, free eTau levels were reduced to 69.3% baseline, 31.5% baseline, 12.8% baseline and to the lower limit of quantitation following 0.5, 2, 5 and 20 mg/kg IV doses, respectively. Free eTau levels were reduced to a minimum levels of 69.3% by 72 hrs, 26.7% by 168 hrs, 11.5% by 336 hrs and <10% by 24 hrs (% baseline) following 0.5, 2, 5 and 20 mg/kg IV doses, respectively. In contrast, free eTau levels in the vehicle-dosed animal (n=1) varied between 89.9% and 130.2%. Maximal reduction of free CSF eTau following 0.5 mg/kg was ˜50% while 20 mg/kg produced >90% reduction and intermediate doses produced values within this range. Reductions in free eTau were long lasting and were still observed at 1512 hr (57 days) post dose in the 2, 5 and 20 mg/kg dose groups though levels were trending back to baseline. Free CSF eTau returned to baseline levels 8 days and 57 days following the 0.5 and 2 mg/kg doses, respectively, while free CSF eTau was still suppressed by ˜50% and ˜70% at 57 days following the 5 and 20 mg/kg doses, respectively. These results confirm the pharmacodynamic activity of hu-IPN002 in CSF. The reductions in free eTau observed could be explained by multiple mechanisms including hu-IPN002 binding to eTau, a reduction in the absolute levels of eTau or a combination of both.

Analysis of Aβ Levels in CSF

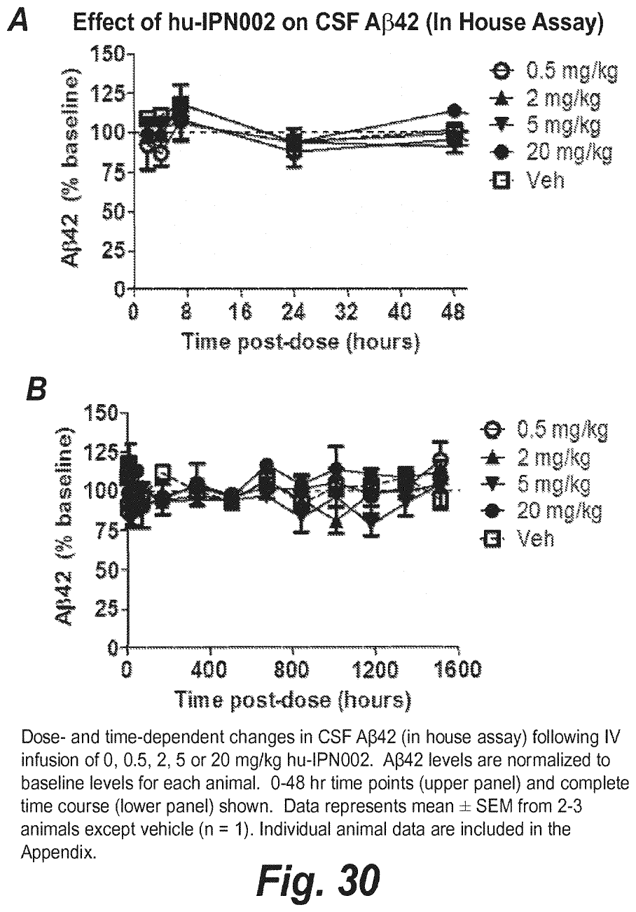

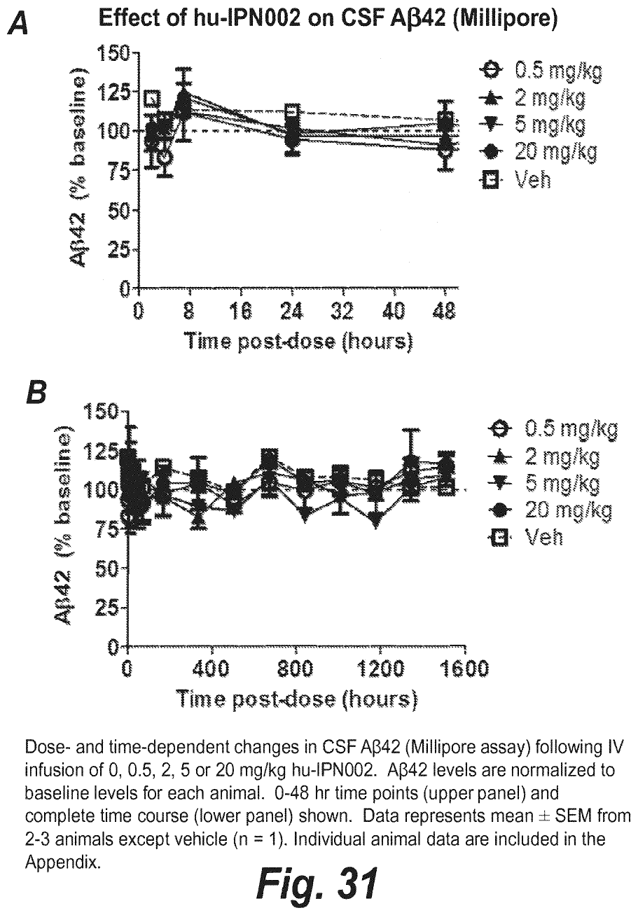

The effect of hu-IPN002 on Aβ42 levels in CSF was measured using two different sandwich ELISAs, including an in-house assay and a commercial kit (Millipore). As shown in FIGS. 30A-30B and Table 7 below (in-house assay) and in FIGS. 31A-31B and Table 8 below (Millipore assay), hu-IPN002 did not affect CSF Aβ42 levels at any dose. This is not consistent with the other experiments described herein. While the basis for this discrepancy is unclear, it could be due to different dosing regimens (e.g., multiple dose versus single dose protocols).

TABLE 7
Effect of hu-IPN002 on CSF Aβ42 (In house Assay)

| Time | | 0.5 | | | | | | | | | 20 | | |
|---|---|---|---|---|---|---|---|---|---|---|---|---|---|
| post- | | mpk | | | 2 mpk | | | 5 mpk | | | mpk | | |
| dose | Veh | (n = 3) | | | (n = 2) | | | (n = 2) | | | (n = 2) | | |
| (hr) | (n = 1) | Mean | SD | CV | Mean | SD | CV | Mean | SD | CV | Mean | SD | CV |
| 2.0 | 108.5 | 92.2 | 26.5 | 29 | 97.4 | 2.1 | 2 | 103.7 | 7.8 | 8 | 98.8 | 0.8 | 1 |
| 4.0 | 110.7 | 86.8 | 13.8 | 16 | 102.1 | 10.5 | 10 | 103.7 | 3.0 | 3 | 98.1 | 3.1 | 3 |
| 7.0 | 117.5 | 107.8 | 20.9 | 19 | 117.9 | 4.8 | 4 | 105.6 | 16.0 | 15 | 118.0 | 17.4 | 15 |
| 24.0 | 93.5 | 87.4 | 16.3 | 19 | 93.6 | 2.1 | 2 | 93.4 | 4.1 | 4 | 93.3 | 12.3 | 13 |
| 48.0 | 100.8 | 94.8 | 14.1 | 15 | 98.9 | 5.8 | 6 | 90.4 | 2.1 | 2 | 113.2 | 6.1 | 5 |
| 72.0 | 95.8 | 91.1 | 25.0 | 27 | 103.1 | 2.5 | 2 | 99.7 | 2.3 | 2 | 89.8 | 0.8 | 1 |
| 168 | 111.6 | 94.5 | 16.5 | 17 | 93.2 | 2.7 | 3 | 94.1 | 4.2 | 4 | 96.4 | 1.2 | 1 |
| 336 | 99.8 | 104.4 | 23.3 | 22 | 94.2 | 0.4 | 0 | 96.7 | 0.5 | 0 | 102.8 | 0.4 | 0 |
| 504 | 93.2 | 97.1 | 7.2 | 7 | 94.7 | 5.8 | 6 | 94.6 | 2.9 | 3 | 96.2 | 0.9 | 1 |
| 672 | 107.3 | 101.6 | 11.8 | 12 | 106.3 | 7.2 | 7 | 96.6 | 1.2 | 1 | 116.4 | 3.6 | 3 |
| 840 | 91.7 | 101.0 | 15.8 | 16 | 94.3 | 7.8 | 8 | 82.9 | 12.9 | 16 | 104.1 | 6.8 | 7 |
| 1008 | 102.5 | 105.4 | 15.2 | 14 | 80.9 | 11.5 | 14 | 96.0 | 8.4 | 9 | 113.8 | 20.0 | 18 |
| 1176 | 102.6 | 97.2 | 12.2 | 13 | 98.2 | 21.8 | 22 | 78.0 | 9.7 | 12 | 110.3 | 2.9 | 3 |
| 1344 | 108.6 | 101.8 | 9.7 | 10 | 99.9 | 7.5 | 7 | 92.1 | 11.7 | 13 | 108.4 | 8.9 | 8 |
| 1512 | 93.9 | 119.2 | 20.6 | 17 | 103.3 | 21.8 | 21 | 103.9 | 7.6 | 7 | 111.7 | 10.7 | 10 |

TABLE 8
Effect of hu-IPN002 on CSF Aβ42 (Millipore)

| Time | | 0.5 | | | | | | | | | 20 | | |
|---|---|---|---|---|---|---|---|---|---|---|---|---|---|
| post- | | mpk | | | 2 mpk | | | 5 mpk | | | mpk | | |
| dose | Veh | (n = 3) | | | (n = 2) | | | (n = 2) | | | (n = 2) | | |
| (hr) | (n = 1) | Mean | SD | CV | Mean | SD | CV | Mean | SD | CV | Mean | SD | CV |
| 2.0 | 120.5 | 93.6 | 28.8 | 31 | 97.1 | 0.8 | 1 | 98.7 | 15.9 | 16 | 101.6 | 0.0 | 0 |
| 4.0 | 106.4 | 83.2 | 19.5 | 23 | 102.0 | 0.8 | 1 | 103.5 | 11.2 | 11 | 103.5 | 4.3 | 4 |
| 7.0 | 113.6 | 111.9 | 32.0 | 29 | 124.7 | 20.8 | 17 | 112.3 | 1.6 | 1 | 121.0 | 0.5 | 0 |
| 24.0 | 112.0 | 94.7 | 16.8 | 18 | 97.0 | 15.7 | 16 | 101.5 | 4.3 | 4 | 98.0 | 7.2 | 7 |
| 48.0 | 106.9 | 87.9 | 21.5 | 24 | 97.0 | 11.0 | 11 | 91.0 | 0.4 | 0 | 104.8 | 19.8 | 19 |
| 72.0 | 102.3 | 92.1 | 23.6 | 26 | 95.2 | 2.1 | 2 | 107.3 | 0.6 | 1 | 89.5 | 12.5 | 14 |
| 168 | 114.1 | 97.6 | 25.3 | 26 | 95.1 | 0.9 | 1 | 96.7 | 18.7 | 19 | 104.3 | 15.0 | 14 |
| 336 | 107.6 | 104.0 | 28.8 | 28 | 82.9 | 11.1 | 13 | 89.4 | 11.8 | 2 | 104.1 | 8.1 | 8 |
| 504 | 98.6 | 90.5 | 8.7 | 10 | 104.2 | 6.0 | 6 | 86.5 | 5.7 | 7 | 98.2 | 5.9 | 6 |
| 672 | 120.9 | 104.8 | 16.5 | 16 | 110.9 | 15.2 | 14 | 106.8 | 14.5 | 14 | 119.3 | 7.9 | 7 |
| 840 | 108.3 | 99.7 | 20.7 | 21 | 106.6 | 8.7 | 8 | 83.5 | 1.0 | 1 | 105.2 | 8.2 | 8 |
| 1008 | 108.2 | 105.7 | 16.2 | 15 | 96.3 | 2.7 | 3 | 94.6 | 14.0 | 15 | 105.4 | 11.1 | 11 |
| 1176 | 106.2 | 101.5 | 13.6 | 13 | 98.2 | 19.3 | 20 | 79.1 | 1.1 | 1 | 97.0 | 3.6 | 4 |
| 1344 | 101.6 | 111.8 | 14.7 | 13 | 107.0 | 12.1 | 11 | 101.7 | 12.6 | 12 | 118.2 | 27.3 | 23 |
| 1512 | 101.8 | 114.9 | 15.7 | 14 | 109.3 | 5.5 | 5 | 108.2 | 8.4 | 8 | 116.4 | 7.7 | 7 |

Specifically, as shown in FIGS. 30A-30B, CSF Aβ42 levels varied from 91.7% to 117.5% (% baseline) in the vehicle dosed animal using the house assay. As shown in FIGS. 31A-31B, levels varied from 98.6% to 120.9% (% baseline) using the Millipore assay. In both assays, CSF Aβ42 levels in the hu-IPN002-dosed animals were similar to the vehicle controls.

### Example 6: Effect of hu-IPN002 on Tau Levels and on Aβ levels (Multiple Intravenous Dose Study)

A multiple dose, intravenous (IV) bolus infusion study was conducted to evaluate the pharmacokinetics and pharmacodynamics of hu-IPN002 over a 4-6 month time frame following multiple intravenous doses to male cynomolgus monkeys. Doses were administered on Days 1, 29 and 57 of the study. The doses employed were:
1. 0 mg/kg (vehicle)×3 doses;
2. 20 mg/kg×3 doses;
3. 40 mg/kg×3 doses; and
4. 60 mg/kg×1 dose followed by 20 mg/kg×2 doses.
   The 20 mg/kg×3 dose group was extended for an additional 56 days following the final dose.

hu-IPN002 levels were measured in both serum and CSF samples using ELISA. Blood was obtained from all animals at 0 (predose), 0.05, 0.083, 0.5, 1, 8, 12, 24, 48, 72, 120, 168, 336, 504, 648 hours following dosing on Day 1, at 0 (predose), 0.05, 0.083, 0.25, 4, 8, 12, 24, 48, 96, 168, 336, 504, 648 hours following dosing on Day 29, and at 0 (predose), 0.05, 0.083, 0.5, 1, 8, 12, 24, 48, 72, 120, 168, 336, 504, 672, 840, 1008, 1176, 1344 hours following dosing on Day 57 for analysis of serum hu-IPN002. Additional blood samples were collected at 1512, 1680, 1848, 2016, 2184, 2352, 2520, and 2688 hours following dosing on Day 57 from animals in the 20 mg/kg×3 dose group for analysis of serum hu-IPN002.

Analysis of Serum hu-IPN002 Concentrations

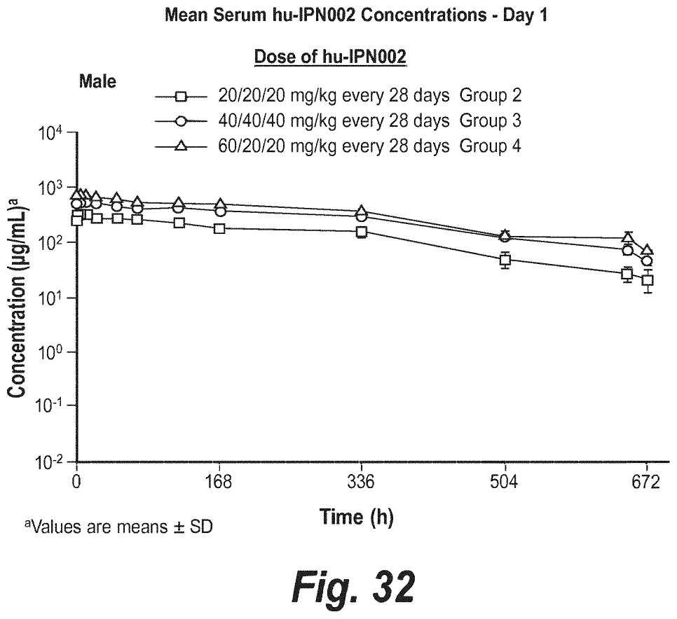

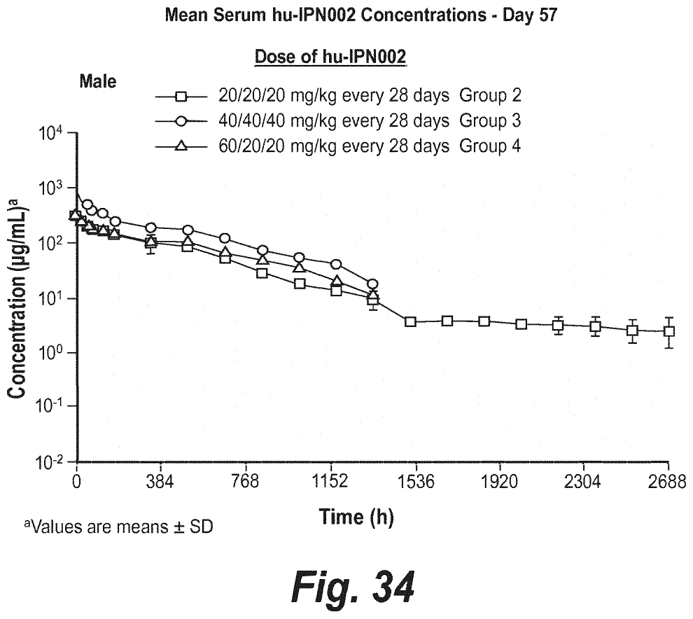

After the first dose, mean hu-IPN002 systemic exposures (AUC[0-672 h]) increased approximately dose proportionally from 20 to 60 mg/kg (Table 9; FIG. 32). After repeated dosing, mean hu-IPN002 systemic exposures (AUC[0-672 h]) on Day 57 (after the third dose) also increased in a dose proportional manner between 20 and 40 mg/kg every 28 days (Table 11; FIG. 34). The mean serum T-HALF values ranged from 210 to 390 hours.

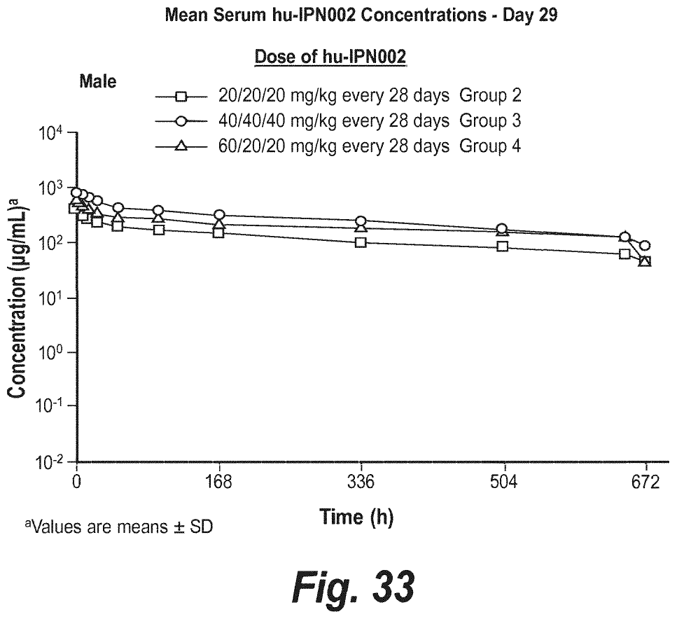

After repeated dosing, at 20 and 40 mg/kg every 28 days, mean hu-IPN002 systemic exposures (AUC[0-672 h]) following the third dose on Day 57 were similar (0.8 and 0.9×) to those after the first dose, and were comparable (1.0 and 0.9×) to the exposures after the second dose on Day 29 (Tables 10 and 11; FIGS. 33 and 34). No accumulation or loss of exposure was observed. Steady state was achieved after the first dose.

After a loading dose of 60 mg/kg and two maintenance doses at 20 mg/kg every 28 days, the mean hu-IPN002 systemic exposure (AUC[0-672 h]) on Day 57 in Group 4 were similar (1.1×) to the exposure in Group 2 following 3 doses at 20 mg/kg every 28 days, indicating the loading dose had no substantial impact on serum hu-IPN002 exposure on Day 57 (Table 11 and FIG. 34).

TABLE 9
Mean Serum hu-IPN002 Pharmacokinetic Parameters- Day 1

| | hu-IPN002 (mg/kg every 28 days) | | |
|---|---|---|---|
| | 20/20/20 | 40/40/40 | 60/20/20 |
| Parameter | Males | | |
| AUC (0-672 h); μg · h/mL | 90,100 | 178,000 | 220,000 |
| Cmax; μg/mL | 328 | 553 | 681 |
| Tmax; h | 5.2 | 2.4 | 3.5 |

TABLE 10
Mean Serum hu-IPN002 Pharmacokinetic Parameters- Day 29

| | hu-IPN002 (mg/kg every 28 days) | | |
|---|---|---|---|
| | 20/20/20 | 40/40/40 | 60/20/20 |
| Parameter | Males | | |
| AUC (0-672 h); μg · h/mL | 76,400 | 169,000 | 125,000 |
| Cmax; μg/mL; | 463 | 880 | 483 |
| Tmax; h | 0.066 | 0.11 | 0.11 |

TABLE 11
Mean Serum hu-IPN002 Pharmacokinetic Parameters - Day 57

| | hu-IPN002 (mg/kg every 28 days) | | |
|---|---|---|---|
| | 20/20/20 | 40/40/40 | 60/20/20 |
| Parameter | Males | | |
| AUC (0-672 h); μg · h/mL | 76,200 | 157,000 | 82,400 |
| Cmax; μg/mL | 472 | 947 | 600 |
| Tmax; h | 0.050 | 0.050 | 0.050 |
| T-HALF; h | 390 | 290 | 210 |

For T-HALF, value is harmonic mean.

Analysis of CSF hu-IPN002 Concentrations

CSF was also obtained from all animals prior to dose and at 8, 48, 168, 336, 504, 648 hours following dosing on Days 1 and 29, and at 8, 48, 168, 336, 504, 672, 840, 1008, 1176, and 1344 hours following dosing on Day 57 for analysis of CSF hu-IPN002, Additional CSF samples at 1512, 1680, 1848, 2016, 2184, 2352, 2520, and 2688 hours post dosing on Day 57 were collected from animals in the 20 mg/kg×3 dose group for analysis of CSF hu-IPN002.

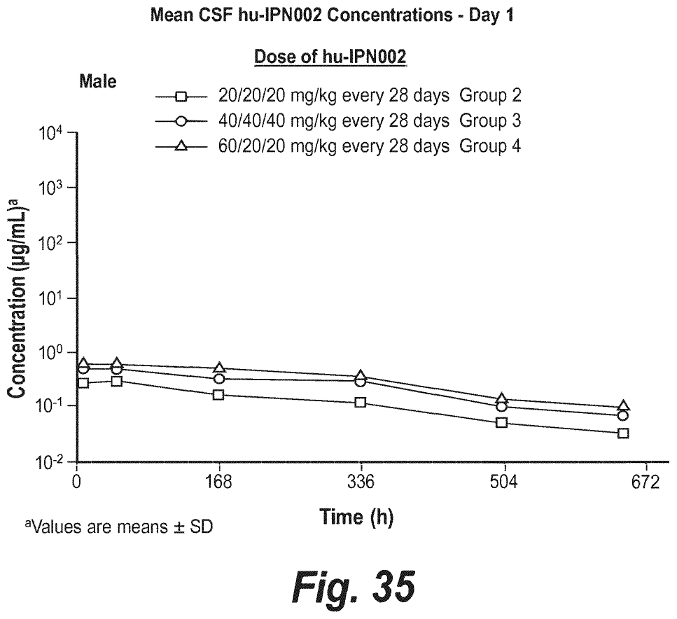

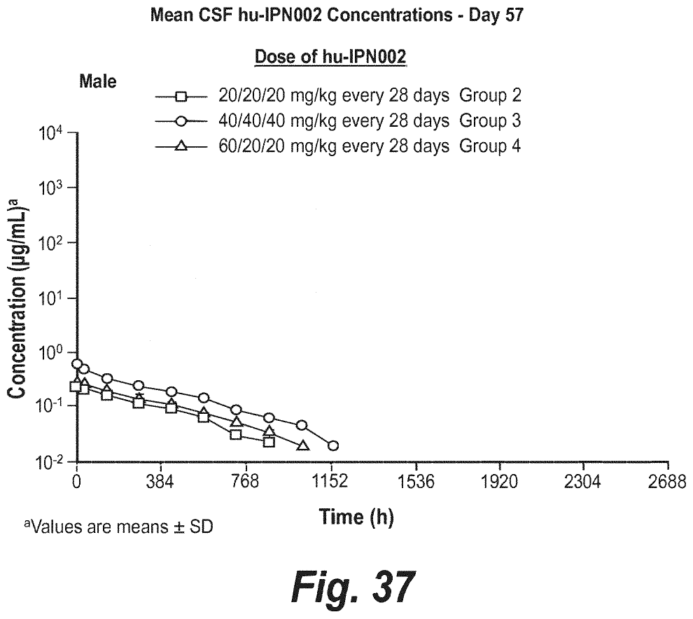

After the first dose, mean CSF hu-IPN002 exposures (AUC[0-T]) increased approximately dose proportionally from 20 to 60 mg/kg (Table 12; FIG. 35). After repeated dosing, mean CSF hu-IPN002 exposures (AUC[0-672 h]) on Day 57 also increased in a dose proportional manner from 20 to 40 mg/kg every 28 days (Table 14; FIG. 37)), with CSF AUC (0-672 h) values that were 0.0013 to 0.0014× the corresponding serum AUC (0-672 h) values. Mean Tmax values were reached at 8 hours post dose. The mean CSF apparent T-HALF values ranged from 250 to 310 hours. Following the third dose on Day 57, the mean CSF/Serum AUC (0-672h) ratios were 0.0013 to 0.0014.

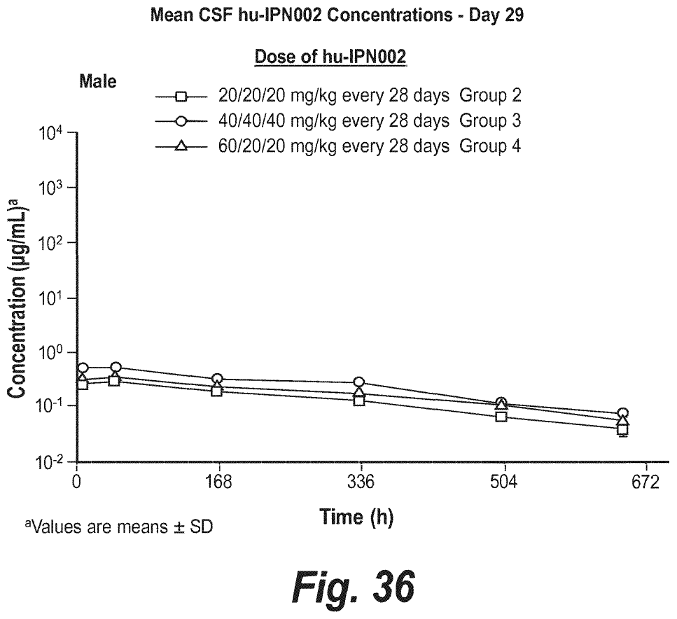

After repeated dosing at 20 and 40 mg/kg every 28 days, mean CSF hu-IPN002 exposures (AUC[0-672 h]) following the third dose on Day 57 were similar (1.2×) to those after the first dose, and were comparable (1.1 and 1.2×) to the exposures after the second dose on Day 29 (Table 13; FIG. 36). No accumulation or loss of exposure was observed. Steady state was achieved after the first dose.

After a loading dose of 60 mg/kg followed by two 20 mg/kg/every 4 weeks doses, the mean CSF hu-IPN002 exposure (AUC[0-672 h]) on Day 57 in Group 4 were similar (1.2×) to the exposure in Group 2 which received 20 mg/kg/every 28 days dose, indicating loading dose had no substantial impact on serum hu-IPN002 exposure (Table 14: FIG. 37). The apparent CSF T-HALF values ranged from 250 to 310 hours.

hu-IPN002 was not observed in any control CSF samples.

TABLE 12
Mean CSF hu-IPN002 Pharmacokinetic Parameters - Day 1

| | hu-IPN002 (mg/kg every 28 days) | | |
|---|---|---|---|
| | 20/20/20 | 40/40/40 | 60/20/20 |
| Parameter | Males | | |
| AUC (0-648 h)^{a}; μg · h/mL | 84.3 | 166 | 223 |
| Cmax; μg/mL | 0.308 | 0.498 | 0.618 |
| Tₘₐₓ; h | 38 | 28 | 28 |
| CSF/Serum AUC(0-T) Ratio^{b} | 0.00095 | 0.00093 | 0.0010 |

^{a}AUC (0-T) values were truncated to AUC (0-648 h) due to no sample was collected at 672 hours post dose.
^{b}CSF/Serum AUC (0-T) = CSF AUC (0-648 h)/Serum AUC (0-672 h); the ratios may be lower than expected if the 672-hour CSF samples were collected.

TABLE 13
Mean CST hu-IPN002 Pharmacokinetic Parameters - Day 29

| | hu-IPN002 (mg/kg every 28 days) | | |
|---|---|---|---|
| | 20/20/20 | 40/40/40 | 60/20/20 |
| Parameter | Males | | |
| AUC (0-648)^{a}; μg · h/mL | 91.8 | 170 | 117 |
| Cmax; μg/mL | 0.294 | 0.519 | 0.339 |
| Tmax; h | 38 | 28 | 38 |
| CSF/Serum AUC(0-T) Ratio^{b} | 0.0012 | 0.0011 | 0.00082 |

^{a}AUC (0-T) values were truncated to AUC (0-648 h) due to no sample was collected at 672 hours post dose.
^{b}CSF/Serum AUC (0-T) = CSF AUC (0-648 h)/Serum AUC (0-672 h); the ratios may be lower than expected if the 672-hour CSF samples were collected.

TABLE 14
Mean CSF hu-IPN002 Pharmacokinetic Parameters - Day 57

| | hu-IPN002 (mg/kg every 28 days) | | |
|---|---|---|---|
| | 20/20/20 | 40/40/40 | 60/20/20 |
| Parameter | Males | | |
| AUC (0-672 h); μg · h/mL | 97.1 | 199 | 114 |
| Cmax; μg/mL | 0.373 | 0.642 | 0.402 |
| Tmax; h | 8.0 | 8.0 | 8.0 |
| T-HALF; h | 280 | 310 | 250 |
| CSF/Serum AUC (0-672 h) Ratio | 0.0013 | 0.0013 | 0.0014 |

For T-HALF, value is harmonic mean

Analysis of Free eTan Levels in CSF

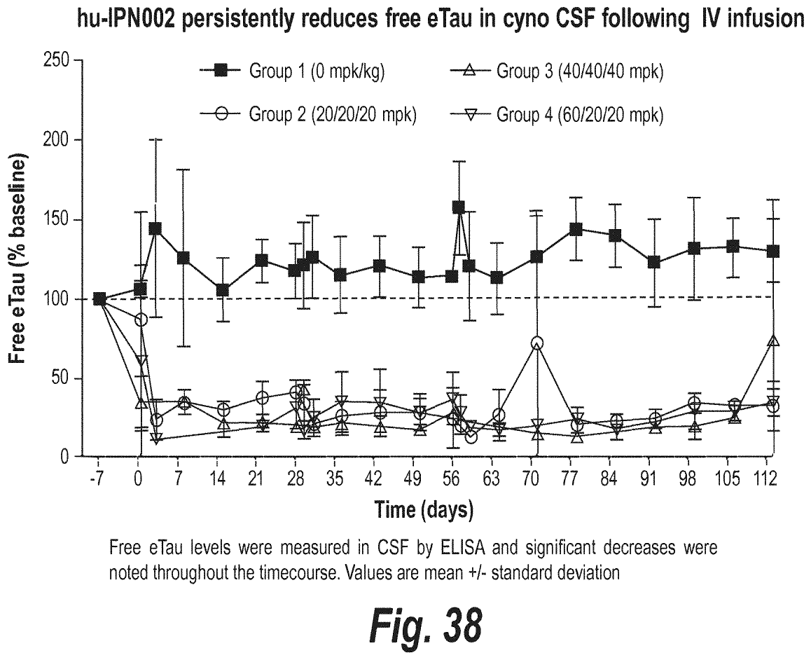

The effect of hu-IPN002 on free eTau levels in CSF was measured using a commercially available ELISA. The CSF free eTau levels (percentage of baseline)versus time profile for all doses is shown in FIG. 38.

After the first dose. CSF free eTau levels were rapidly reduced by all three doses in a dose-dependent manner at the earliest time point at the earliest time point measured, 8 hours. CSF free eTau levels appeared maximally suppressed at the 40 mg/kg dose but all doses reduced CSF free eTau by ≥75%. Free eTau levels did not return to baseline for any dose group up to day 112 of the study or 55 days following the last dose.

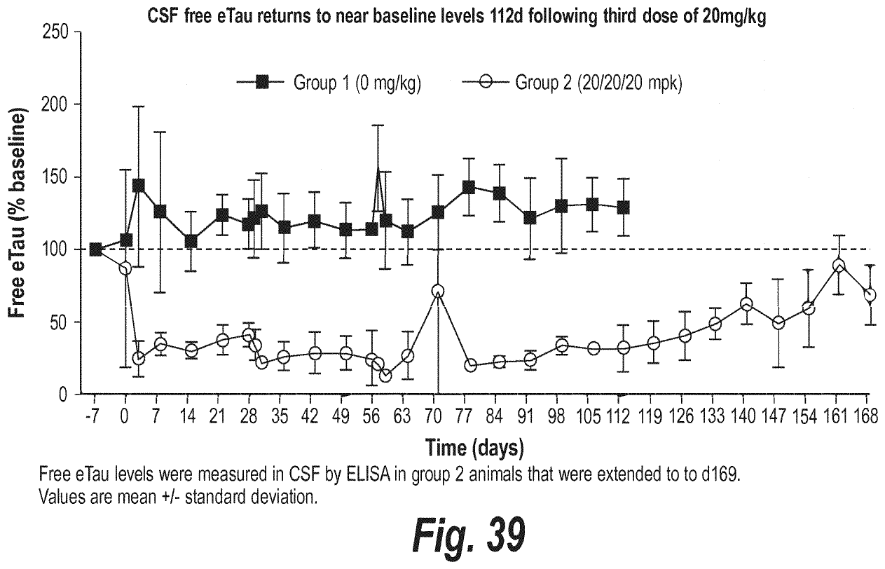

The study was extended by 2 months for the 20 mg/kg dose to determine if CSF free eTau levels would return to baseline. As seen in FIG. 39, by study day 162-169 or 105-112 days following the last of 3 doses, CSF free eTau returns to near baseline values. At most of the time points at the 3 doses the reductions in eTau were significantly different from vehicle (data not shown). In some cases p-values could not be calculated because assay values were below the limit of detection.

In sum, hu-IPN002 produced rapid and sustained decreases in free eTau in cyno CSF following IV infusion in this repeated dosing, multiple dose level study. At all dose levels studied, CSF free eTau remained suppressed for the study duration (112 days) or 55 days following the third dose. The 40 mg/kg dose level appeared to provide the most sustained reduction of CSF free eTau.

Analysis of Aβ42 Levels in CSF

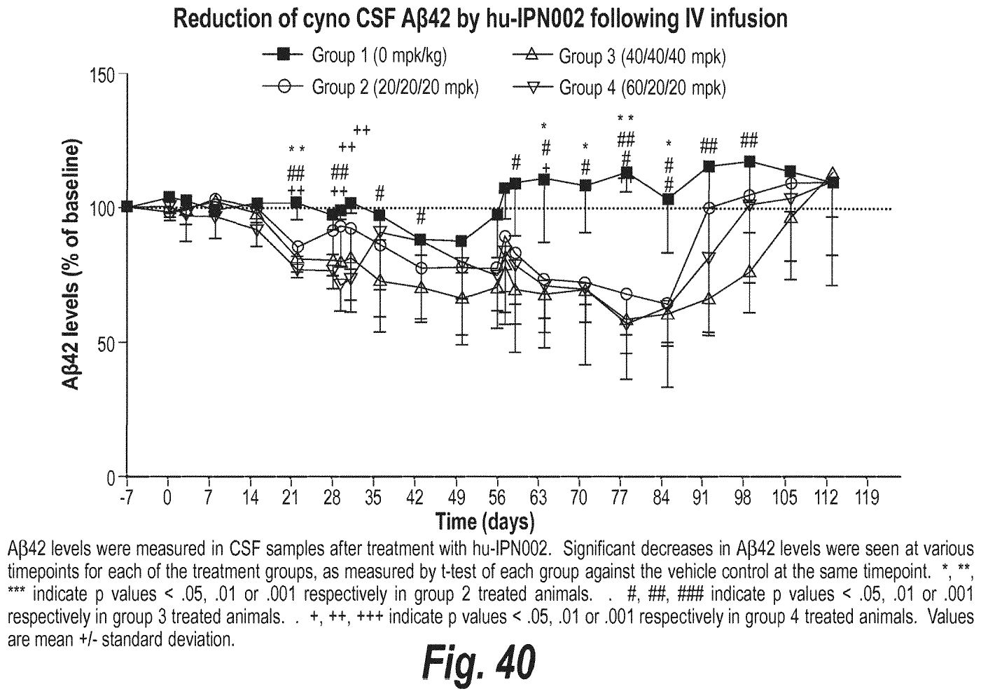

The CSF Aβ42 levels (percentage of baseline) versus time profile for all doses are showed in FIG. 40. As shown in FIG. 40, hu-IPN002 reduced CSF Aβ2 in this study. All doses reduced CSF Aβ42 21 days following the first dose. The greatest and most sustained (40-50 days) reduction in Aβ42 began following the third dose (day 57). Maximal reduction of Aβ42 (25-50% of baseline) occurred at study day 77 or 20 days following the third dose. At all dose levels, Aβ42 values returned to baseline by study day 106 or 49 days following the third dose. There was modest dose-dependence to CSF Aβ42 lowering produced by hu-IPN002.

### Example 6: Prediction of Pharmacokinetics and Efficacious Doses in Humans

Estimation of Pharmacokinetics

The human pharmacokinetic parameters for Ihu-IPN002 were predicted based on single species allometry from the monkey. The human clearance was predicted to be 0.06 mL/h/kg. The predicted volume of distribution at steady state (Vss) in humans is 0.041 L/kg.

To capture the bi-exponential nature of the serum concentration profile seen in monkeys, the human pharmacokinetic profile was predicted using the Css-mean residence time (MRT) method. Non-compartmental analysis of the predicted human profile generated a volume (Vz) and half-life (T-HALF) of 0.04 L/kg and 535 h, respectively. The estimated pharmacokinetic parameters are set forth in Table 15 below.

TABLE 15
Predicted Human parameters for hu-IPN002 from monkey

| | Monkey | Predicted |
|---|---|---|
| | PK parameters | Human parameters |
| Vss (L/kg) | 0.041 | 0.041 |
| CLTp (mL/h/kg) | 0.11 | 0.06 |
| Vz (L/kg) (from NCA analysis) | 0.043 | 0.04 |
| T-HALF (h) (from NCA analysis) | 275 | 535 |
| Vc (L/kg) | 0.027 | 0.025 |
| k12 (h⁻¹) | 0.025 | 0.023 |
| k21 (h⁻¹) | 0.03 | 0.023 |
| ke (h⁻¹) | 0.004 | 0.002 |

The model predicted kdeg (0.1 h⁻¹) differed from the reported literature value for half-life of tau (11 days equivalent to kdeg=0.002 h−1) in the CSF (Yamada. K, et al., J. Exp. Med., 2014 Mar. 10; 211(3):387-93).

Prediction of Efficacious Doses in Humans

A sustained depression of 75% in the eTau concentrations for 4 weeks was selected to give a target engagement most likely to be efficacious in humans.

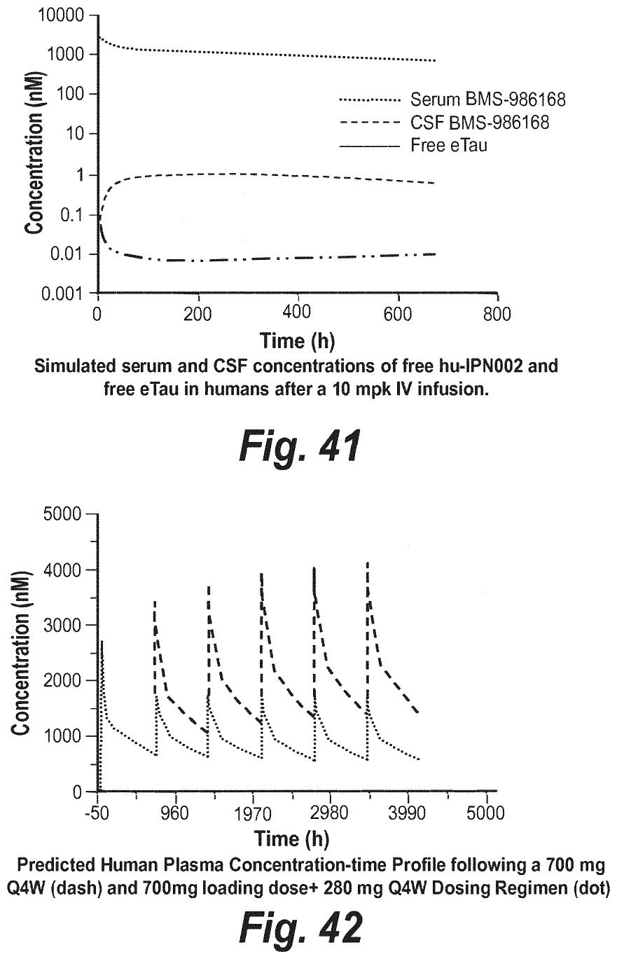

A dose of 10 mg/kg (700 mg) is needed to achieve 75% reduction in eTau over 4 weeks. The predicted concentration-time profile for hu-IPN002 in serum and CSF and eTau in CSF were also simulated (FIG. 41).

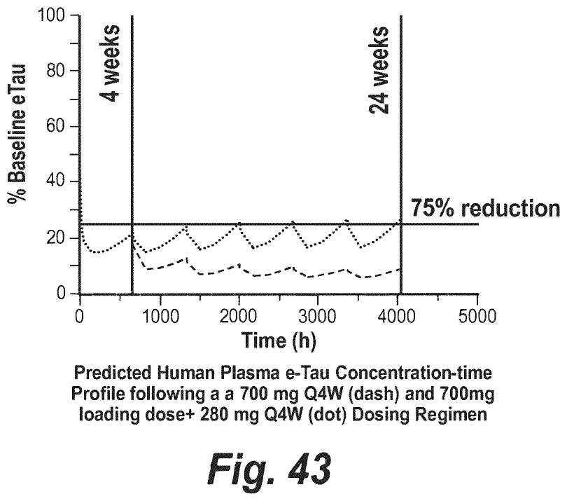

In the steady state simulations, a 10 mg/kg dose administered once every 4 weeks, was predicted to sustain reduction in free eTau concentrations over 24 weeks. The overall serum exposure of hu-IPN002 may be reduced and the % eTau reduction maintained at or above 75% by the administration of a 10 mg/kg loading dose, followed by a maintenance dose of 4 mg/kg, administered every 4 weeks (FIGS. 42-43). The predicted Cmaxₛₛ for 10 mg/kg Q4W was calculated to be 592 ug/ml, while that for 10 mg/kg loading dose followed by 4 mg/kg Q4W was found to be 241 ug/ml. The corresponding AUCₛₛ for the two dosing regimen are 204,977 μg*h/mL and 84,114 μg*h/mL, respectively. A loading and maintenance dose approach is expected to allow substantial reduction of free eTau levels immediately proximal to dosing with a single loading dose and sustain the reduction in eTau levels using lower maintenance doses.

TABLE 16

PK parameters at steady state

| | | AUC (TAU)* |
|---|---|---|
| Dosing regimen | Cmax (μg/ml) | (μg · h/mL) |
| 700 mg Q4W | 592 | 204,977 |
| 700 mg + 280 mg | 241 | 84,114 |
| Q4W | | |

*AUC (TAU) is the AUC over the dosing interval.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### SUMMARY OF SEQUENCE LISTING

| SEQ | |
|---|---|
| ID | |
| NO: | SEQUENCE |
| 1 | RSSQTILHSNGNTYLE |
| 2 | KVSKRFS |
| 3 | FQGSLVPWA |
| 4 | SYGMS |
| 5 | TISSSGSRTYFPDSVKG |
| 6 | TWDGAMDY |
| 7 | KSSQSIVHSNGNTYLE |
| 8 | KVSNRFS |
| 9 | FQGSLVPWA |
| 10 | KYGMS |
| 11 | TISSSGSRTYYPDSVKG |
| 12 | SWDGAMDY |
| 13 | [Hybridoma IPN001 Light Chain amino acid]; |
| | FIG. 11B |
| 14 | [Hybridoma IPN001 Heavy Chain amino acid]; |
| | FIG. 11A |
| 15 | [Hybridoma IPN002 Light Chain amino acid]; |
| | FIG. 12B |
| 16 | [Hybridoma IPN002 Heavy Chain amino acid]; |
| | FIG. 12A |
| 17 | [Hybridoma IPN001 Light Chain nucleotide]; |
| | FIG. 11B |
| 18 | [Hybridoma IPN001 Heavy Chain nucleotide] |
| | FIG. 11A |
| 19 | [Hybridoma IPN002 Light Chain nucleotide]; |
| | FIG. 12B |
| 20 | [Hybridoma IPN002 Heavy Chain nucleotide]; |
| | FIG. 12A |
| 21 | EFEVMED |
| 22 | DQGGYT |
| 23 | MAEPRQEFEVMEDHAGTY |
| 24 | AGTYGLGDRK |
| 25 | EVX1LVESGGALVKPGGSLRLSCAASGFSFS |
| 26 | WVRQAPGKGLEWVA |
| 27 | RFTISRDNAKNTLYLQMX2SX3X4X5EDTAMYYCX6I: |
| 28 | [IPN002 VH Variant 1 nucleotide]; |
| | FIG. 13 |
| 29 | [IPN002 VH Variant 2 nucleotide]; |
| | FIG. 14 |
| 30 | [IPN002 VH Variant 3 nucleotide]; |
| | FIG. 15 |
| 31 | [IPN002 VH Variant 4 nucleotide]; |
| | FIG. 16 |
| 32 | [IPN002 Vk Variant 1 nucleotide]; |
| | FIG. 17 |
| 33 | [IPN002 Vk Variant 2 nucleotide]; |
| | FIG. 18 |
| 34 | [IPN002 Vk Variant 3 nucleotide]; |
| | FIG. 19 |
| 35 | [IPN002 Vk Variant 4 nucleotide]; |
| | FIG. 20 |
| 36 | [IPN002 VH Valiant 1 amino acid]; |
| | FIG. 13 |
| 37 | [IPN002 VH Variant 2 amino acid] |
| | FIG. 14 |
| 38 | [IPN002 VH Variant 3 amino acid]; |
| | FIG. 15 |
| 39 | [IPN002 VH Variant 4 amino acid]; |
| | FIG. 16 |
| 40 | [IPN002 Vk Variant 1 amino acid]; |
| | FIG. 17 |
| 41 | [IPN002 Vk Variant 2 amino acid]; |
| | FIG. 18 |
| 42 | [IPN002 Vk Variant 3 amino acid]; |
| | FIG. 19 |
| 43 | [IPN002 Vk Variant 4 amino acid]; |
| | FIG. 20 |
| 44 | WGQGTX7VTVSS |
| 45 | DVX1MTQSPLSLPVTLGQPASISC |
| 46 | WYLQKPGQSPQLLX2Y |
| 47 | GVPDRFSGSGSGTDFTLKISRVEAEDVGX3YYC |
| 48 | FGGGTKVEIK |
| 49 | (GSGGS)n |
| 50 | (GGGS)n |
| 51 | GGSG |
| 52 | GGSGG |
| 53 | GSGSG |
| 54 | GSGGG |
| 55 | GGGSG |
| 56 | GSSSG |
| 57 | HHHHHH |
| 58 | YPYDVPDYA |
| 59 | DYKDDDDK |
| 60 | EQKLISEEDL |
| 61 | HHHHH |
| 62 | WSHQFEK |
| 63 | RYIRS |
| 64 | FHHT |
| 65 | WEAAAREACCRECCARA |
| 66 | TFFYGGCRGKRNNFKTEEY |
| 67 | TFFYGGSRGKRNNFKTEEY |
| 68 | CTFFYGGSRGKRNNFKTEEY |
| 69 | TFFYGGSRGKRNNFKTEEYC |
| 70 | TFVYGGCRAKRNNFKS |
| 71 | eTau 4; FIG. 9 |
| 72 | 2N4R; FIG. 9 |
| 73 | Fetal extracellular Tau polypeptide; |
| | FIG. 21 |
| 74 | Extracellular Tau polypeptide #2; FIG. 21 |
| 75 | Extracellular Tau polypeptide #3; FIG. 21 |
| 76 | Extracellular Tau polypeptide #4; FIG. 21 |
| 77 | eTau 2-172; FIG. 21 |
| 78 | eTau 2-176; FIG. 21 |

1. A method of treating an acute tauopathy in an individual, the method comprising administering to the individual an anti-Tau antibody in an amount effective to reduce significantly the level of free Tau in an extracellular fluid of the individual.

2.-29. (canceled)

30. A method of treating traumatic brain injury or stroke in an individual, the method comprising administering to the individual an anti-Tau antibody in an amount effective to reduce significantly the level of free Tau in an extracellular fluid of the individual.

31.-58. (canceled)

59. A method for treating a tauopathy in a human subject in need thereof, the method comprising administering to the human subject a fixed dose of 700 mg of an anti-tau antibody, wherein the anti-tau antibody comprises an immunoglobulin light chain variable region (VL) and an immunoglobulin heavy chain variable region (VH), wherein: the VH consists of the amino acid sequence set forth in SEQ ID NO:37 and the VL consists of the amino acid sequence set forth in SEQ ID NO:41.

60. The method of claim 59, wherein the anti-tau antibody is administered to the human subject intravenously.

61. The method of claim 59, wherein the anti-tau antibody is administered to the human subject at a fixed dose of 700 mg once every four weeks.

62. The method of claim 59, wherein the tauopathy is Alzheimer's disease.

63. The method of claim 59, wherein the chronic tauopathy is progressive supranuclear palsy.

64. A method for treating a tauopathy in a human subject in need thereof, the method comprising administering an anti-tau antibody to the human subject in a single bolus injection, wherein the anti-tau antibody comprises an immunoglobulin light chain variable region (VL) and an immunoglobulin heavy chain variable region (VH), wherein: the VH consists of the amino acid sequence set forth in SEQ ID NO:37 and the VL consists of the amino acid sequence set forth in SEQ ID NO:41.

65. The method of claim 64, wherein the anti-tau antibody is administered in multiple doses.

66. The method of claim 64, wherein any two doses of the anti-tau antibody are administered within 5 days, 7 days, 2 weeks, 4 weeks, 2 months or more of one another.

67. The method of claim 64, wherein the anti-tau antibody is administered by subcutaneous administration, by intrathecal administration, or by intravenous administration.

68. The method of claim 64, wherein the tauopathy is Alzheimer's disease, amyotrophic lateral sclerosis/parkinsonism dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tangle only dementia, or multi-infarct dementia.

69. The method of claims 64, wherein the tauopathy is Alzheimer's disease.

70. The method of claims 64, wherein the tauopathy is progressive supranuclear palsy.

71. The method of claims 64, wherein the tauopathy is stroke, chronic traumatic encephalopathy, traumatic brain injury, concussion, seizure, epilepsy, or acute lead encephalopathy.

72. A method for treating a tauopathy in a human subject in need thereof, the method comprising administering any two doses of an anti-tau antibody to the human subject within 5 days, 4 weeks or 2 months of one another, wherein the anti-tau antibody comprises an immunoglobulin light chain variable region (VL) and an immunoglobulin heavy chain variable region (VH), wherein: the VH consists of the amino acid sequence set forth in SEQ ID NO:37 and the VL consists of the amino acid sequence set forth in SEQ ID NO:41.

73. The-method of claim 72, wherein the anti-tau antibody is administered by subcutaneous administration, by intrathecal administration, or by intravenous administration.

74. The method of claim 72, wherein the tauopathy is Alzheimer's disease, amyotrophic lateral sclerosis/parkinsonism dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tangle only dementia, or multi-infarct dementia.

75. The method of claims 72, wherein the tauopathy is Alzheimer's disease.

76. The method of claims 72, wherein the tauopathy is progressive supranuclear palsy.

77. The method of claims 72, wherein the tauopathy is stroke, chronic traumatic encephalopathy, traumatic brain injury, concussion, seizure, epilepsy, or acute lead encephalopathy.


SEQUENCE LISTING (79 sequences)

SEQ ID NO: 1 - protein, 16 aa, Artificial Sequence
  source: /note=“Description of Artificial Sequence Synthetic peptide”
RSSQTILHSNGNTYLE

SEQ ID NO: 2 - protein, 7 aa, Artificial Sequence
  source: /note=“Description of Artificial Sequence Synthetic peptide”
KVSKRFS

SEQ ID NO: 3 - protein, 9 aa, Artificial Sequence
  source: /note=“Description of Artificial Sequence Synthetic peptide”
FQGSLVPWA

SEQ ID NO: 4 - protein, 5 aa, Artificial Sequence
  source: /note=“Description of Artificial Sequence Synthetic peptide”
SYGMS

SEQ ID NO: 5 - protein, 17 aa, Artificial Sequence
  source: /note=“Description of Artificial Sequence Synthetic peptide”
TISSSGSRTYFPDSVKG

SEQ ID NO: 6 - protein, 8 aa, Artificial Sequence
  source: /note=“Description of Artificial Sequence Synthetic peptide”
TWDGAMDY

SEQ ID NO: 7 - protein, 16 aa, Artificial Sequence
  source: /note=“Description of Artificial Sequence Synthetic peptide”
KSSQSIVHSNGNTYLE

SEQ ID NO: 8 - protein, 7 aa, Artificial Sequence
  source: /note=“Description of Artificial Sequence Synthetic peptide”
KVSNRFS

SEQ ID NO: 9 - protein, 9 aa, Artificial Sequence
  source: /note=“Description of Artificial Sequence Synthetic peptide”
FQGSLVPWA

SEQ ID NO: 10 - protein, 5 aa, Artificial Sequence
  source: /note=“Description of Artificial Sequence Synthetic peptide”
KYGMS

SEQ ID NO: 11 - protein, 17 aa, Artificial Sequence
  source: /note=“Description of Artificial Sequence Synthetic peptide”
TISSSGSRTYYPDSVKG

SEQ ID NO: 12 - protein, 8 aa, Artificial Sequence
  source: /note=“Description of Artificial Sequence Synthetic peptide”
SWDGAMDY

SEQ ID NO: 13 - protein, 112 aa, Artificial Sequence
  source: /note=“Description of Artificial Sequence Synthetic polypeptide”
DVLMTQTPLSLAVNLGDQASLSCRSSQTILHSNGNTYLEWYLQKPGQSPRLLIYKVSKRFSGVPDRFSGSGSGTDFTLKISRVEADDLGIYYCFQGSLVPWAFGGGTKLEIK

SEQ ID NO: 14 - protein, 117 aa, Artificial Sequence
  source: /note=“Description of Artificial Sequence Synthetic polypeptide”
EVQLVESGEDLVKPGGSLKLSCVASGFAFSSYGMSWVRQTPDMRLEWVATISSSGSRTYFPDSVKGRLTISRDNDKNILYLQMSSLRSEDTAMYYCTITWDGAMDYWGRGISVTVSS

SEQ ID NO: 15 - protein, 112 aa, Artificial Sequence
  source: /note=“Description of Artificial Sequence Synthetic polypeptide”
DVLMTQTPLSLPVSLGDQASISCKSSQSIVHSNGNTYLEWYLQKPGQSPKLLVYKVSNRFSGVPDRFSGSGSGTDFTLKISRVEAEDLGTYYCFQGSLVPWAFGGGTKLEIK

SEQ ID NO: 16 - protein, 117 aa, Artificial Sequence
  source: /note=“Description of Artificial Sequence Synthetic polypeptide”
EVHLVESGGALVKPGGSLKLSCAASGFSFSKYGMSWVRQTPDKRLEWVATISSSGSRTYYPDSVKGQFTISRDNAKNTLYLQMSSLKSEDTAMYYCSISWDGAMDYWGQGTSVTVSS

SEQ ID NO: 17 - DNA, 336 bp, Artificial Sequence
  source: /note=“Description of Artificial Sequence Synthetic polynucleotide”
  CDS (1)..(336)
gatgttttgatgacccaaactccgctctccctggcagtcaatcttggagatcaagcctccctctcttgcagatcgagtcagactattttacatagtaatggaaatacctatttagaatggtatttgcagaaaccaggccagtctccaagactcctgatctacaaagtttctaaacgattttctggggtcccagacaggttcagtggcagtggatcagggacagatttcacactcaagatcagcagagtggaggctgacgatctgggaatttattactgctttcaaggttcacttgttccttgggcgttcggtggaggcaccaagctggaaatcaaa

SEQ ID NO: 18 - DNA, 351 bp, Artificial Sequence
  source: /note=“Description of Artificial Sequence Synthetic polynucleotide”
  CDS (1)..(351)
gaggtgcagttggtggagtctggggaagacttagtgaagcctggagggtccctgaaactctcctgtgtcgcttctggattcgctttcagtagctatggcatgtcttgggttcgccagactccagacatgaggctggagtgggtcgcaacaattagtagcagtggtagtcgcacctactttccagacagtgtgaaggggcgactcaccatctccagagacaatgacaagaacatcctatacctacaaatgagcagtctgaggtctgaggacacagccatgtactattgtacgattacctgggacggtgctatggactactggggtcgtggaatatcagtcaccgtctcctca

SEQ ID NO: 19 - DNA, 336 bp, Artificial Sequence
  source: /note=“Description of Artificial Sequence Synthetic polynucleotide”
  CDS (1)..(336)
gatgttttgatgacccaaactccactctccctgcctgtcagtcttggagatcaagcctccatctcttgcaaatctagtcagagcattgtacatagtaatggaaacacctatttagaatggtacctgcagaaaccaggccagtctccaaagctcctggtctacaaagtttccaatcgattttctggggtcccagacaggttcagtggcagtggatcagggacagatttcacactcaagatcagcagagtggaggctgaggatctgggaacttattactgctttcaaggttcacttgttccttgggcgttcggtggaggcaccaagctggaaatcaaa

SEQ ID NO: 20 - DNA, 351 bp, Artificial Sequence
  source: /note=“Description of Artificial Sequence Synthetic polynucleotide”
  CDS (1)..(351)
gaggttcatctggtggagtctgggggagccttagtgaagcctggagggtccctgaaactctcctgtgcagcctctggattcagtttcagtaaatatggcatgtcttgggttcgccagactccagacaagaggctggagtgggtcgcaaccattagtagtagtgggagtcgcacctactatccagacagtgtgaagggccaattcaccatctccagagacaatgccaagaacaccctgtacctgcaaatgagcagtctgaagtctgaggacacagccatgtattactgttcaattagctgggacggtgctatggactactggggtcaagggacctcagtcaccgtctcctca

SEQ ID NO: 21 - protein, 7 aa, Artificial Sequence
  source: /note=“Description of Artificial Sequence Synthetic peptide”
EFEVMED

SEQ ID NO: 22 - protein, 6 aa, Artificial Sequence
  source: /note=“Description of Artificial Sequence Synthetic peptide”
DQGGYT

SEQ ID NO: 23 - protein, 18 aa, Artificial Sequence
  source: /note=“Description of Artificial Sequence Synthetic peptide”
MAEPRQEFEVMEDHAGTY

SEQ ID NO: 24 - protein, 10 aa, Artificial Sequence
  source: /note=“Description of Artificial Sequence Synthetic peptide”
AGTYGLGDRK

SEQ ID NO: 25 - protein, 30 aa, Artificial Sequence
  source: /note=“Description of Artificial Sequence Synthetic polypeptide”
  VARIANT (3)..(3): /replace=“Gln”
  MISC_FEATURE (1)..(30): /note=“Variant residue given in the sequence has no preference with respect to that in the annotation for variant position”
EVHLVESGGALVKPGGSLRLSCAASGFSFS

SEQ ID NO: 26 - protein, 14 aa, Artificial Sequence
  source: /note=“Description of Artificial Sequence Synthetic peptide”
WVRQAPGKGLEWVA

SEQ ID NO: 27 - protein, 32 aa, Artificial Sequence
  source: /note=“Description of Artificial Sequence Synthetic polypeptide”
  VARIANT (18)..(18): /replace=“Asn”
  VARIANT (20)..(20): /replace=“Leu”
  VARIANT (21)..(21): /replace=“Arg”
  VARIANT (22)..(22): /replace=“Ala”
  VARIANT (31)..(31): /replace=“Ala”
  MISC_FEATURE (1)..(32): /note=“Variant residues given in the sequence have no preference with respect to those in the annotations for variant positions”
RFTISRDNAKNTLYLQMSSSKSEDTAMYYCSI

SEQ ID NO: 28 - DNA, 351 bp, Artificial Sequence
  source: /note=“Description of Artificial Sequence Synthetic polynucleotide”
  CDS (1)..(351)
gaggttcatctggtggagtctgggggagccttagtgaagcctggagggtccctgagactctcctgtgcagcctctggattcagtttcagtaaatatggcatgtcttgggttcgccaggccccaggcaagggcctggagtgggtcgcaaccattagtagtagtgggagtcgcacctactatccagacagtgtgaagggcagattcaccatctccagagacaatgccaagaacaccctgtacctgcaaatgagcagtctgaagtctgaggacacagccatgtattactgttcaattagctgggacggtgctatggactactggggtcaagggacctcagtcaccgtctcctca

SEQ ID NO: 29 - DNA, 351 bp, Artificial Sequence
  source: /note=“Description of Artificial Sequence Synthetic polynucleotide”
  CDS (1)..(351)
gaggttcatctggtggagtctgggggagccttagtgaagcctggagggtccctgagactctcctgtgcagcctctggattcagtttcagtaaatatggcatgtcttgggttcgccaggccccaggcaagggcctggagtgggtcgcaaccattagtagtagtgggagtcgcacctactatccagacagtgtgaagggcagattcaccatctccagagacaatgccaagaacaccctgtacctgcaaatgaacagtctgagagccgaggacacagccatgtattactgttcaattagctgggacggtgctatggactactggggtcaagggaccaccgtcaccgtctcctca

SEQ ID NO: 30 - DNA, 351 bp, Artificial Sequence
  source: /note=“Description of Artificial Sequence Synthetic polynucleotide”
  CDS (1)..(351)
gaggttcagctggtggagtctgggggagccttagtgaagcctggagggtccctgagactctcctgtgcagcctctggattcagtttcagtaaatatggcatgtcttgggttcgccaggccccaggcaagggcctggagtgggtcgcaaccattagtagtagtgggagtcgcacctactatccagacagtgtgaagggcagattcaccatctccagagacaatgccaagaacaccctgtacctgcaaatgaacagtctgagagccgaggacacagccatgtattactgttcaattagctgggacggtgctatggactactggggtcaagggaccaccgtcaccgtctcctca

SEQ ID NO: 31 - DNA, 351 bp, Artificial Sequence
  source: /note=“Description of Artificial Sequence Synthetic polynucleotide”
  CDS (1)..(351)
gaggttcagctggtggagtctgggggagccttagtgaagcctggagggtccctgagactctcctgtgcagcctctggattcagtttcagtaaatatggcatgtcttgggttcgccaggccccaggcaagggcctggagtgggtcgcaaccattagtagtagtgggagtcgcacctactatccagacagtgtgaagggcagattcaccatctccagagacaatgccaagaacaccctgtacctgcaaatgaacagtctgagagccgaggacacagccatgtattactgtgccattagctgggacggtgctatggactactggggtcaagggaccaccgtcaccgtctcctca

SEQ ID NO: 32 - DNA, 336 bp, Artificial Sequence
  source: /note=“Description of Artificial Sequence Synthetic polynucleotide”
  CDS (1)..(336)
gatgttttgatgacccaaagcccactctccctgcctgtcacccttggacagcccgcctccatctcttgcaaatctagtcagagcattgtacatagtaatggaaacacctatttagaatggtacctgcagaaaccaggccagtctccacagctcctggtctacaaagtttccaatcgattttctggggtcccagacagattcagtggcagtggatcagggacagatttcacactcaagatcagcagagtggaggctgaggatgtgggaacttattactgctttcaaggctcacttgttccttgggcgttcggtggaggcaccaaggtggaaatcaaa

SEQ ID NO: 33 - DNA, 336 bp, Artificial Sequence
  source: /note=“Description of Artificial Sequence Synthetic polynucleotide”
  CDS (1)..(336)
gatgttgtgatgacccaaagcccactctccctgcctgtcacccttggacagcccgcctccatctcttgcaaatctagtcagagcattgtacatagtaatggaaacacctatttagaatggtacctgcagaaaccaggccagtctccacagctcctggtctacaaagtttccaatcgattttctggggtcccagacagattcagtggcagtggatcagggacagatttcacactcaagatcagcagagtggaggctgaggatgtgggaacttattactgctttcaaggctcacttgttccttgggcgttcggtggaggcaccaaggtggaaatcaaa

SEQ ID NO: 34 - DNA, 336 bp, Artificial Sequence
  source: /note=“Description of Artificial Sequence Synthetic polynucleotide”
  CDS (1)..(336)
gatgttgtgatgacccaaagcccactctccctgcctgtcacccttggacagcccgcctccatctcttgcaaatctagtcagagcattgtacatagtaatggaaacacctatttagaatggtacctgcagaaaccaggccagtctccacagctcctggtctacaaagtttccaatcgattttctggggtcccagacagattcagtggcagtggatcagggacagatttcacactcaagatcagcagagtggaggctgaggatgtgggagtgtattactgctttcaaggctcacttgttccttgggcgttcggtggaggcaccaaggtggaaatcaaa

SEQ ID NO: 35 - DNA, 336 bp, Artificial Sequence
  source: /note=“Description of Artificial Sequence Synthetic polynucleotide”
  CDS (1)..(336)
gatgttgtgatgacccaaagcccactctccctgcctgtcacccttggacagcccgcctccatctcttgcaaatctagtcagagcattgtacatagtaatggaaacacctatttagaatggtacctgcagaaaccaggccagtctccacagctcctgatctacaaagtttccaatcgattttctggggtcccagacagattcagtggcagtggatcagggacagatttcacactcaagatcagcagagtggaggctgaggatgtgggagtgtattactgctttcaaggctcacttgttccttgggcgttcggtggaggcaccaaggtggaaatcaaa

SEQ ID NO: 36 - protein, 117 aa, Artificial Sequence
  source: /note=“Description of Artificial Sequence Synthetic polypeptide”
EVHLVESGGALVKPGGSLRLSCAASGFSFSKYGMSWVRQAPGKGLEWVATISSSGSRTYYPDSVKGRFTISRDNAKNTLYLQMSSLKSEDTAMYYCSISWDGAMDYWGQGTSVTVSS

SEQ ID NO: 37 - protein, 117 aa, Artificial Sequence
  source: /note=“Description of Artificial Sequence Synthetic polypeptide”
EVHLVESGGALVKPGGSLRLSCAASGFSFSKYGMSWVRQAPGKGLEWVATISSSGSRTYYPDSVKGRFTISRDNAKNTLYLQMNSLRAEDTAMYYCSISWDGAMDYWGQGTTVTVSS

SEQ ID NO: 38 - protein, 117 aa, Artificial Sequence
  source: /note=“Description of Artificial Sequence Synthetic polypeptide”
EVQLVESGGALVKPGGSLRLSCAASGFSFSKYGMSWVRQAPGKGLEWVATISSSGSRTYYPDSVKGRFTISRDNAKNTLYLQMNSLRAEDTAMYYCSISWDGAMDYWGQGTTVTVSS

SEQ ID NO: 39 - protein, 117 aa, Artificial Sequence
  source: /note=“Description of Artificial Sequence Synthetic polypeptide”
EVQLVESGGALVKPGGSLRLSCAASGFSFSKYGMSWVRQAPGKGLEWVATISSSGSRTYYPDSVKGRFTISRDNAKNTLYLQMNSLRAEDTAMYYCAISWDGAMDYWGQGTTVTVSS

SEQ ID NO: 40 - protein, 112 aa, Artificial Sequence
  source: /note=“Description of Artificial Sequence Synthetic polypeptide”
DVLMTQSPLSLPVTLGQPASISCKSSQSIVHSNGNTYLEWYLQKPGQSPQLLVYKVSNRFSGVPDRFSGSGSGTDFTLKISRVEAEDVGTYYCFQGSLVPWAFGGGTKVEIK

SEQ ID NO: 41 - protein, 112 aa, Artificial Sequence
  source: /note=“Description of Artificial Sequence Synthetic polypeptide”
DVVMTQSPLSLPVTLGQPASISCKSSQSIVHSNGNTYLEWYLQKPGQSPQLLVYKVSNRFSGVPDRFSGSGSGTDFTLKISRVEAEDVGTYYCFQGSLVPWAFGGGTKVEIK

SEQ ID NO: 42 - protein, 112 aa, Artificial Sequence
  source: /note=“Description of Artificial Sequence Synthetic polypeptide”
DVVMTQSPLSLPVTLGQPASISCKSSQSIVHSNGNTYLEWYLQKPGQSPQLLVYKVSNRFSGVPDRFSGSGSGTDFTLKISRVEAEDVGVYYCFQGSLVPWAFGGGTKVEIK

SEQ ID NO: 43 - protein, 112 aa, Artificial Sequence
  source: /note=“Description of Artificial Sequence Synthetic polypeptide”
DVVMTQSPLSLPVTLGQPASISCKSSQSIVHSNGNTYLEWYLQKPGQSPQLLIYKVSNRFSGVPDRFSGSGSGTDFTLKISRVEAEDVGVYYCFQGSLVPWAFGGGTKVEIK

SEQ ID NO: 44 - protein, 11 aa, Artificial Sequence
  source: /note=“Description of Artificial Sequence Synthetic peptide”
  VARIANT (6)..(6): /replace=“Thr”
  MISC_FEATURE (1)..(11): /note=“Variant residue given in the sequence has no preference with respect to that in the annotation for variant position”
WGQGTSVTVSS

SEQ ID NO: 45 - protein, 23 aa, Artificial Sequence
  source: /note=“Description of Artificial Sequence Synthetic peptide”
  VARIANT (3)..(3): /replace=“Val”
  MISC_FEATURE (1)..(23): /note=“Variant residue given in the sequence has no preference with respect to that in the annotation for variant position”
DVLMTQSPLSLPVTLGQPASISC

SEQ ID NO: 46 - protein, 15 aa, Artificial Sequence
  source: /note=“Description of Artificial Sequence Synthetic peptide”
  VARIANT (14)..(14): /replace=“Ile”
  MISC_FEATURE (1)..(15): /note=“Variant residue given in the sequence has no preference with respect to that in the annotation for variant position”
WYLQKPGQSPQLLVY

SEQ ID NO: 47 - protein, 32 aa, Artificial Sequence
  source: /note=“Description of Artificial Sequence Synthetic polypeptide”
  VARIANT (29)..(29): /replace=“Val”
  MISC_FEATURE (1)..(32): /note=“Variant residue given in the sequence has no preference with respect to that in the annotation for variant position”
GVPDRFSGSGSGTDFTLKISRVEAEDVGTYYC

SEQ ID NO: 48 - protein, 10 aa, Artificial Sequence
  source: /note=“Description of Artificial Sequence Synthetic peptide”
FGGGTKVEIK

SEQ ID NO: 49 - protein, 5 aa, Artificial Sequence
  source: /note=“Description of Artificial Sequence Synthetic peptide”
GSGGS

SEQ ID NO: 50 - protein, 4 aa, Artificial Sequence
  source: /note=“Description of Artificial Sequence Synthetic peptide”
GGGS

SEQ ID NO: 51 - protein, 4 aa, Artificial Sequence
  source: /note=“Description of Artificial Sequence Synthetic peptide”
GGSG

SEQ ID NO: 52 - protein, 5 aa, Artificial Sequence
  source: /note=“Description of Artificial Sequence Synthetic peptide”
GGSGG

SEQ ID NO: 53 - protein, 5 aa, Artificial Sequence
  source: /note=“Description of Artificial Sequence Synthetic peptide”
GSGSG

SEQ ID NO: 54 - protein, 5 aa, Artificial Sequence
  source: /note=“Description of Artificial Sequence Synthetic peptide”
GSGGG

SEQ ID NO: 55 - protein, 5 aa, Artificial Sequence
  source: /note=“Description of Artificial Sequence Synthetic peptide”
GGGSG

SEQ ID NO: 56 - protein, 5 aa, Artificial Sequence
  source: /note=“Description of Artificial Sequence Synthetic peptide”
GSSSG

SEQ ID NO: 57 - protein, 6 aa, Artificial Sequence
  source: /note=“Description of Artificial Sequence Synthetic 6xHis tag”
HHHHHH

SEQ ID NO: 58 - protein, 9 aa, Artificial Sequence
  source: /note=“Description of Artificial Sequence Synthetic peptide”
YPYDVPDYA

SEQ ID NO: 59 - protein, 8 aa, Artificial Sequence
  source: /note=“Description of Artificial Sequence Synthetic peptide”
DYKDDDDK

SEQ ID NO: 60 - protein, 10 aa, Artificial Sequence
  source: /note=“Description of Artificial Sequence Synthetic peptide”
EQKLISEEDL

SEQ ID NO: 61 - protein, 5 aa, Artificial Sequence
  source: /note=“Description of Artificial Sequence Synthetic 5xHis tag”
HHHHH

SEQ ID NO: 62 - protein, 8 aa, Artificial Sequence
  source: /note=“Description of Artificial Sequence Synthetic peptide”
WSHPQFEK

SEQ ID NO: 63 - protein, 5 aa, Artificial Sequence
  source: /note=“Description of Artificial Sequence Synthetic peptide”
RYIRS

SEQ ID NO: 64 - protein, 4 aa, Artificial Sequence
  source: /note=“Description of Artificial Sequence Synthetic peptide”
FHHT

SEQ ID NO: 65 - protein, 17 aa, Artificial Sequence
  source: /note=“Description of Artificial Sequence Synthetic peptide”
WEAAAREACCRECCARA

SEQ ID NO: 66 - protein, 19 aa, Artificial Sequence
  source: /note=“Description of Artificial Sequence Synthetic peptide”
TFFYGGCRGKRNNFKTEEY

SEQ ID NO: 67 - protein, 19 aa, Artificial Sequence
  source: /note=“Description of Artificial Sequence Synthetic peptide”
TFFYGGSRGKRNNFKTEEY

SEQ ID NO: 68 - protein, 20 aa, Artificial Sequence
  source: /note=“Description of Artificial Sequence Synthetic peptide”
CTFFYGGSRGKRNNFKTEEY

SEQ ID NO: 69 - protein, 20 aa, Artificial Sequence
  source: /note=“Description of Artificial Sequence Synthetic peptide”
TFFYGGSRGKRNNFKTEEYC

SEQ ID NO: 70 - protein, 16 aa, Artificial Sequence
  source: /note=“Description of Artificial Sequence Synthetic peptide”
TFVYGGCRAKRNNFKS

SEQ ID NO: 71 - protein, 67 aa, Artificial Sequence
  source: /note=“Description of Artificial Sequence Synthetic polypeptide”
AEPRQEFEVMEDHAGTYGLGDRKDQGGYTMHQDQEGDTDAGLKAEEAGIGDTPSLEDEAAGHVTQAR

SEQ ID NO: 72 - protein, 441 aa, Artificial Sequence
  source: /note=“Description of Artificial Sequence Synthetic polypeptide”
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 73 - protein, 352 aa, Artificial Sequence
  source: /note=“Description of Artificial Sequence Synthetic polypeptide”
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 74 - protein, 150 aa, Artificial Sequence
  source: /note=“Description of Artificial Sequence Synthetic polypeptide”
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSR

SEQ ID NO: 75 - protein, 121 aa, Artificial Sequence
  source: /note=“Description of Artificial Sequence Synthetic polypeptide”
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPK

SEQ ID NO: 76 - protein, 67 aa, Artificial Sequence; identical to SEQ ID NO: 71
  source: /note=“Description of Artificial Sequence Synthetic polypeptide”

SEQ ID NO: 77 - protein, 171 aa, Artificial Sequence
  source: /note=“Description of Artificial Sequence Synthetic polypeptide”
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVR

SEQ ID NO: 78 - protein, 175 aa, Artificial Sequence
  source: /note=“Description of Artificial Sequence Synthetic polypeptide”
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPK

SEQ ID NO: 79 - protein, 10 aa, Artificial Sequence
  source: /note=“Description of Artificial Sequence Synthetic peptide”
  MISC_FEATURE (1)..(10): /note=“This sequence may encompass 2-10 repeating Gly residues”
GGGGGGGGGG